SEQUENCE LISTING (46 sequences)

SEQ ID NO: 1 - DNA, 16 bp, Artificial Sequence
  Nucleic acid silencing molecule targeting AASS
  misc_feature (1)..(1): n is t or u
  misc_feature (10)..(12): n is t or u
ncagaaagannnacgc

SEQ ID NO: 2 - DNA, 16 bp, Artificial Sequence
  nucleic acid silencing molecule targeting AASS
  misc_feature (1)..(1): n is t or u
  misc_feature (5)..(5): n is t or u
  misc_feature (8)..(8): n is t or u
  misc_feature (10)..(10): n is t or u
  misc_feature (16)..(16): n is t or u
naggnaancngaaggn

SEQ ID NO: 3 - DNA, 16 bp, Artificial Sequence
  nucleic acid silencing molecule targeting AASS
  misc_feature (7)..(9): n is t or u
  misc_feature (16)..(16): n is t or u
gaaagannnacgccan

SEQ ID NO: 4 - DNA, 16 bp, Artificial Sequence
  nucleic acid silencing molecule targeting AASS
  misc_feature (5)..(6): n is t or u
cgggnnaggaggcgag

SEQ ID NO: 5 - DNA, 16 bp, Artificial Sequence
  nucleic acid silencing molecule targeting AASS
  misc_feature (3)..(3): n is t or u
  misc_feature (5)..(5): n is t or u
  misc_feature (9)..(9): n is t or u
  misc_feature (12)..(12): n is t or u
ccngngccnaangaag

SEQ ID NO: 6 - DNA, 16 bp, Artificial Sequence
  nucleic acid silencing molecule targeting AASS
  misc_feature (1)..(1): n is t or u
  misc_feature (3)..(3): n is t or u
  misc_feature (9)..(9): n is t or u
  misc_feature (15)..(15): n is t or u
ngnagcgcnccggang

SEQ ID NO: 7 - DNA, 20 bp, Artificial Sequence
  nucleic acid silencing molecule targeting AASS
  misc_feature (7)..(8): n is t or u
ggcgggnnaggaggcgagga

SEQ ID NO: 8 - DNA, 20 bp, Artificial Sequence
  nucleic acid silencing molecule targeting AASS
  misc_feature (1)..(1): n is t or u
  misc_feature (3)..(3): n is t or u
  misc_feature (5)..(5): n is t or u
  misc_feature (11)..(11): n is t or u
  misc_feature (17)..(17): n is t or u
  misc_feature (19)..(20): n is t or u
nangnagcgcnccggangnn

SEQ ID NO: 9 - DNA, 72701 bp, Homo sapiens
  misc_feature (1001)..(1089): Exon 1
  misc_feature (11509)..(11733): Exon 2
  misc_feature (15713)..(15889): Exon 3
  misc_feature (18791)..(18875): Exon 4
  misc_feature (26620)..(26687): Exon 5
  misc_feature (26797)..(26943): Exon 6
  misc_feature (28293)..(28371): Exon 7
  misc_feature (28490)..(28617): Exon 8
  misc_feature (30028)..(30176): Exon 9
  misc_feature (31530)..(31652): Exon 10
  misc_feature (32021)..(32132): Exon 11
  misc_feature (43570)..(43629): Exon 12
  misc_feature (43812)..(43879): Exon 13
  misc_feature (46384)..(46505): Exon 14
  misc_feature (46674)..(46800): Exon 15
  misc_feature (52092)..(52202): Exon 16
  misc_feature (52299)..(52407): Exon 17
  misc_feature (53407)..(53547): Exon 18
  misc_feature (59071)..(59238): Exon 19
  misc_feature (63655)..(63750): Exon 20
  misc_feature (65538)..(65653): Exon 21
  misc_feature (66300)..(66388): Exon 22
  misc_feature (67236)..(67412): Exon 23
  misc_feature (68643)..(71701): Exon 24
agaagaaaattccaactctacttacagataaaattctaatttggggttgggcacagtggctaacacctgtaatcccagtgctttgggaggacgaggtgggaggatagcttgaggctaggagttccagaccagtgtcctggtcaacacagcgagaccccatgtctaccacaaataaataaataaataattttctctgcaattggagatatgagtggagtctcaagaacgttctggctatagattgttgacataggagttcagtagctaaatacatctatggcaggtataagaagagtaagtggtggagaaagttgtaaaaacaggagggaagattaacttctgaccgttgacgccttggggaatcacagctgggcttcagacgattcttcagttcctagtgctggggctagtgtctatgaaatacgttttccccctccctccaggcctcctaggtagttaggacttttcaataacattggacattgccatatgcacatgaacacactcagttttaatttctcagattctccgcttgctggcttctaaaactgttgtaagaactgttatctggcttcacagggttgtgacaaggcggtgaaatgctttgaaaagtgtctatagctaaacaaacgtgaggtaccggcgctcgcaacttttccccaggagatgtgcacctctatttagagcgtaatgcccagactattttcgttgctttccgtccccgcccgccttctgccacggacagttacaaacctggtcgataattatcctgggagaactcgacgtcccagctaagaacgcaggtcagagatcagagatcagagacagaaggatcttggggaggagcaggggccgggcatgacgtagcaactggcagcgccgccgattggcagatgagaaggttgttccctcagggcggctgctgattggctacgggaggcagaggcgtgacgtcctcagaagttgagcaaacacctcctacttcccggccttccatcccagtttcttctaggaattcggagcctcccctgcagcgactcggaagattcgaggcggcgggggacaagtcggcgccccagagcggacgaggtgaggagaggagaggtccccgcggagagctgagcagacgcgcgggtgggctcagcctgtgagatgcggtggagatagcgggccgggctcgggctgtcgctgggaggggcccgcgagcctggccttcgggtcccggcgccgcgaggcgaccccggccgggccgagagagggagaaggcggcgagcattcgggcctccctgcgaggaatggagggagagcggaaacgcagagagtttcactgcgaggaagggagggagagcggtcagcgcagctccagcatttctaagtagaaaggcgagggattgcaattcggatttcttttctttcttttttggcggacagtggtggtgctgtggctggggaataaagaactgatgaagctgaggaaacaacggcttgttaatttgtggacggcgtgcttagaggggacggctggctgagggggcggtggatggagatttcggggtggttctttcccccttccctccccacgcccagcttagccaaactgatcacaaacttctctccttgtccatcttgggctgcccactctccgagacacagacccattctccagcaaggatcggagatgggtggaagttgggcgccttgcaagtctgggtttcctgaactcttcagcccctgcgggactatgcagcccattgtgagcgctcgcgcgggacggggcgcggggagaagcagtttacccaccctcctcggttgaatggggctgaatgtctgtctatcctgacagcttcagaagaagaaatgtgcttttcagacattgatttcaggggaatgggggtggggggagcggaagagagagagagaaagagcgctcgagcgcgagagagaatgaattagttcagtaacttgaacaaggattaacattttttgatgttgtgggttatcagtggggaaggtgcagctgtgagatgtagttatacacaggacggaatggtccttgactgaagctgcggctttccaatactgtattaaaaaaaatcccaaaacactgatggcactttggtgtttctgggtttgtctgttttgtcccctgcactcccctcttctttttttcccatgaagatgagtgaatctagggctacatgacttgactgtgtaacctggtggccattctgcaaaatgagttaggtggacaaaaattgaagccaatttacctgaatcattttcccactcttcagataatcatatttgttatcagtttcacttgttattgatgatggcgtgatcagaactttggtttatttgtcattgaattttttttctcactagtcgtttaacagaccttggttcggaattttggaaatgatctttggtgtttaagaagccaccatttaacaaaacattttcatttttttcatatattcttttattaattttctcatagaaataacattacaaacagtggtaagttttcttgggaaggctacaaaagattctatgcaattcatgaaatcatggaaacaaacatttggaatggaaaataaccccaaaacattattaccacattcatctgatctaaggtgtcatctattgtaagatgcatccctatttcactgatgctaaaatgtgtaaaaaaattgtcttaaatttgatgaaatacaacattaaaatggtaatttatttatcttgaacatgcatgcctgaagtttgaagttgaagagagggcttattgggctctaattattttttttccaaaataatgtaagggtgggggattaatatggagccatcgtgaaggtcttactacagtgtgacagaggaaagaaatgtgagtctactgttagcggcgtggttgtgctcgcttttgaggagtaggcaagcctggaggctttttcttttcttaaaatttattgtttatgacacacagcaatgctcagcactttatgcatattagctcaattaaacgtcttaagagccctaagaggtagaggttaatatgattctcattttgcgcatgatgcacagacaggttaagttgccagccaaggtcacagagtaagtttggccgctgaggaacccaggagagaaaccagtattcctttgaggtatccatcttctgttgcaagctcttggttcctggtccatgctattaatgcaggtagcttaggtgaatgtccactctttcttgattgtgtttttggtattttgtttccctgtttggtgtactttagctagaaattcataagtcagagtgcctatttatgttctacatttctaatgtttttgtaattctcagtgtccagttactactatgcttaatagaatcagaatatcagagcctgaaagggactttagtattatatttttgtctgggtaaggaaactgaagtcaagaggaggaaccctacatctagttatgtgtcccagctcccatgtctaagttttcccactatactgtactatatttcacgacatttgttgatctatgagatcttggaagagaatcaagtttttttttttttttttttttttttttgcagggttagggggatgtgagtctcttattaatgtgtctattttatctgtttgtttgtttgtttgttttcttgctgcataggtctttataatgtttattgaatttgctttttcatttattcattctttcttaaaacacttgctgttttcccatgtgccagtcattgggaatacagagatgagtaaaacacaactcatacacatttgcagtttagcaaagagacagacaagtaagcagataagcattttggactagactccgatttagaactaactttgtattccatttcaatgaagctaaaattataagctagagaccaggagaaatatgtgtcatgtttatggatgactttgagtaggtgatgggctggagaaaatatgaaaattctcagaccagattagttaacactaatagacctcctcccccaaagtctaccactttcttcttaatccagtaaagtattttatagagggtttaacatggaagcattaaaagaatgtacccacgcacaggaagtcaaagtatgaccagtgttaatgcaagcccaagcctagttctagcaagccttgaaattgggagggatttggattagttctaggccatcatctggtgctgtgttaattctggaactcttttgaacctctccaaaaaatattatggtttggatacttagtgtaggccaagactcatagccatagagctgccatacttatttacagacaccagtgcttcttgggaaaccatggattttaatatatacttgtattttttttttttttttagttacttggaagggtgaatataaataattagcgctgttcatccttgaacttattttcttcatctaaagactacattacctcattcttagtctctcaatatttaatcacaatatatgcctgattattgcaaataatttgtcattcagaaatcccccgtgtgatttgatagttttggtttgagtcattgaattatgctgtagtgaaattattttaaatatctaaaatagatggacaaagagcacgttcctaactgtggggattaaagttggaacatttggccttaatttcttcaaaaatgaatgtgtgatccttcatacatatcccaaaagaagtaacagaaattaagaggtgcaatgcttagttcattctttttttttttttttttttttttgagactgagtctcgctctgtcgcccaggctggagtgcagtggtacgaactcggctcactgcaagctccgcctcccgggttccacgccattctccagcctcagtctcccgagtagctgggactacaggcgcccgccaccgaccctggctaattttttgtatttttagtagagacggggtttcaccgtgtcagccaggacggtctcgacctcctgacctcgtgatccgcctgcctcagcctcccaaagtgctgggattacaggtgtgagccaccgcacccagccgcttagttcattcttaagctcttgtaatgcttgtgcattgttttgcttaggtaggtgatctataagcttaaaaagtattctgattgggttggtgctatggaaagggttgaagagacttaaagaattttagctatttgatggaagagtaaaaagctctatgaaaatctcagctctcttcatctaccccgttcccttataaaaactgcaaaaattaaatacatcatttgtttgtttgtttgtttgtttgtttgtttgagacagtctcactttgttgtccaggtgggagtgtaatggcatgatcttcacttactgcaaacttcgcctcctgggctcaagtgattttcatgcttcagcctcccgagtagctgggactaccagcatgcgccaccacgcccagttaatttttgtatttttagtagagatggggttttgcatgttgcctaggctggtctcgaactcctcaggtgatctgcccaccctggcctcccaaagtgctggcattataggcataagccactgtgcttggccaaacacatcattttaaggcataaatattgcctgaaaagtagcagagagaaaaatcttgatcgagttacatttaaaaagttataccaacgagaacagacttactgtgacatccattatgataagtcataaaaatacataatagtggcttggaaaatgctttctataaaacagaaaaaaatcccctatttctatgggcctcactattctcatttgtaatactagattttagattacattctatcagtcccactttgactctaatatttggatataatcaataattaataattcactatcagatattgatcagcaccatttaatttgataaaactaatcctgaacatgggcagggattggattactgcttctttatatctttaccaagtgcatcatgccgtgcatatcttaagtagcccccaaatatttttttaattaaagagaatcaaaagcatttgtcccatattccgtgtacaaaagattttattttgataactctatcaagagactttctggatttcattataatttaaatattagaaaatatttgtgctataagcaaatgactagctgtttttatattaatatgtgtaattgtacaaaagaccactttgctcacactctgaaaggacctttctaaccacacatgagattctctgtcctgagagtggcttttcttaaatataatattttataaatacattttacagaatctgctttgctttggttactccatgtgatttagaatgatggtgtactcagtgaccataggacagctcatggtcattgaaagaaaatgggatgtgtatggagtgccgagctctgtctctgtgtccttttttttctctctctctctatctgtctctgtcattttcctttcccttttctggaaaataataaaaaaatacaaagaacattcaagcatgttctttcttctgattctcatgtttttgttgcaaagttagcaaattaaaagattaagtcttgccaccttgagagatggaaagataagacagtatcatagagtatgttctcctattgctactgggcaaccttcactattatcttcctgtctcagtacatagtagttccttcttatccaagttttcgctttccatggtttcagttacccactgtcaatctggtctgaaaataggtgaataaagtacactaagatattgtgagagagaaagagagagtgagagagaccacatttacataacttttattacagtatattgtcataattattctattttattattgttgctagtctcttacagtgcccaatttataaattaaactatatcataggtatgtatgtataggaaaaaacatagtatatatagggtttgggactatcagagatttcaagcatccactgggcttcttggaatgtattacccacagataaacagggtctactgtatctcgctgcaattcactataatactcccatgacttttagagacatttcaaattcctcatcaattttaggtaaataaaaatgtataccttagtggtgttctgttttacaaaaacagagaagaaatcttttaagtttcatttttatatttgaaatgttaaccttcatgtaacaaagggaagactgttacaattcatcagaggaagggtcttgtaccccacccttgcctgtgtgacacttctctttctctattttcctcctctttgttctttctagctctttttttccccctctctgcctcctgcttttctacatcctccttgcttgtgttacaagctgttatcagcacccgacattgtcgggtaacctcactagagctggtggtgagtaaaccctgatcttccctccttccttcattgaaggcattccagaaatatacattgaatttacttcctgatattcttgaaaaataccacaaaatccaggacagaaaaagctaaagcagcctccgttggcatttccatgaaatactagacacctgcagtgcaattcagaaattctcactatttcctatttcttgaagaccttagaagcaaatgaactgtggatcttgaaagttctatgctgagaccaagttttaaggttcaagcagtaactagcagttatactataatcaaaccattttttttctaagcacccagaaatctagaatttatcttggcaggtactcaggaatgattttcagtataaagcaggaaaaggggtcaagagaggcacggcatgctacggggagaactgttaattcactgtgactgttgaaggcaaagccatatggatctgatttctttctgaaacaagagttggctggagttaacagcatgtgttggatctatatggtgtgaatctgtaggctttggcaaagaagagaaaattccaggtgttaaacattaaactaggaatcctgcttctgcttaaaagcaacagatcctatatgattccagttcagtttcttcatctataaaatggggatatgtatctacttaataagatttttgcaaggaggaaataagatgatatccataaagtgctcagcatagagcttggcatgtgatgcttaatcaatggtaactactgttacatttgtttaaataatgaaagttacagaaattattatttaagccttccatgcaaaaagtgaaagtgaaagtagcttcccaaataaactgaagctcagtcccaggcacttctgtctttttccctagtcttccaagatagttggcctttgcaagtatatgtgtgtatgttacaagtgactgtctgtatttgtgtctgtgtgtgtgtctactgctttcccgccctatcccagacaaagaaggtggtgggagaaggagcagggattattactgagtgagggatgagtgaggttaacattcaagcttgggctagaggggagggattaaattatctccacatgttcctcctgatggagtcaagaatttcaactcataataaaagttacaggggtatgtagtcacctgtgaattcatgaaacacagcccctgctgggaagatgggatggtttctggacatttgtgtatgttcaaaggaatcactatctttggaattggttttattagaaactgagggattcaggttctagtttttcttttggcttcgagaagactgaatgttatgggtttgggattgagtttaatgcaatctattttaaaaaacagaagatatgaataaacaaaaaatagatgcagtatataacagtcgtttactgaaatgttgataatctattttgcatgcatattttgccctgaataaaagcctaaactcctaatcatcagcagtttctcttgctgtgtgcagctttttttcttcttggccatcttcctaaagccttcttagctggactcttcgctgtttggtctgttgtctgattctgttgtaaagtgtcctataaatttactgtctaggtagcaagtagttgtttgcccccaggaaatgtacttgtatgcttaattgtttggtcaagccttccccatccatcaagctggtgctcttccaagattgcagtctgatgtaaaataaatgtgtttcatgcaagcttttctatatgttgttagcggtgatgggtagaaaatttctggaaggtataaaaatcattgaatccttatattttgctgccaagtttacatggaaaactaagttttatcattcaaatgatccagcaaatcatcatctgaaatgacatttggctacaaaattgaagttggatagaatccaattgaagttgggtagaatactgttaagcctggatatttatgctttaaaaatttacataagatttattctgcataggaataaatgaacctgtatataaaaattaggagcatctgggtgaaaataaaatgtttactagtgtcactttaatttctttaatttggtttaaagtattatttccaccgtattctacgcttcctccagctgagggaaaaaataactacttttagtagatgatatctttcaatcattctctccttcatatgcagccctggttctgggactagggggacaagacacttcttgtaccaagagaatgctgctttgagtagagtttattcatgagaatataccttgcctgccacattgagagaagcccgttacattcatcatagtaagcctttagtaactatttcttgattgactgaatgagtggaattacacaatcttctcacttgaatagcagttggaaaactggttgaggcaggtcggggtttaatacaacagcagacggccatgtatggagagaaaacccaggaagaggaactggcatgtttagagcaacaaaggtgaacagtgaaggtgagctggactctaggaagcagagtgatttaactctgcttcttaatcttctaacacctctggttaaagaataggtttgtttggtcgattggttttctttttttttttttaatactttaagttctagggtacatgtgcacgacgtgcaggtttgttacagatgtatacatgtgccatgttggtgtgctgcacccgttaactcgtcatttacattaggtgtatctcctaatgctatccctcacccctccccgcaccccatgacaggccccagtgtgtgatgttccccaccctgtgtccaagtgttcccattgttcaattcccacctgtgagtgagaacataaggtgtttggtgttctgtccttgtgatagtttgctcagaatgatggtttccagcttcatccatgtccctacaaaggacatgaactcatccttttttatggctgcatagtattccatggtgtatatgtgccacattttcttaatccagtctatcactgatggacatttgggttggttccaagtctttgctattgtgaatagtgccacaataaatatacatgtgcatgtgtctttatagcagcatgatttctaatcctttgggtatataccagtaatgagatggctgggtcaaatggtatttctagttctagatccttgaggaatcgccacattgtcttccacaatggttgaactagtttacacaggttgattggttttcttaaatttccaatctgttgaggaccaatacttttgtaaaatccggtaacaataaattactagaaaaaatgatcttgtggcttgacatggtggatcacccctgtaatcccagtaatttgttataaattaaaaaaaagaacttagccaggtgggtggttgtgcacctgtagtcctagctactagggaggctgaggctggaggatcacttgagcccagactgcagtaagcaatgatctcaccactgcattttagcattgacagagtgagacttcatgtctaaaaataaataaataaatatgatcatgtgatattaaggcttctaaatgcttactttctatttatgtacacatctaattgtgaaccagtttttttgttttttgttttttgttttaaaaaaacagttcagtgcagttcactgactgacactagggcacactgagaaacacttgttttgggaatggtcagagatgaactggaaagaggttggcactaaattgtgaagaatcttgtataccattataaggatttcaaactttacgccatcaaaatttagaccaatctgaaagtcttgctctttattattttcctggatttgcattgtagattaatttgtttaaaacttttgtgccgaatagatgtttgataaaatgtgtatttaaaaagctattttagcttagcagttattggatggtgtcacacacgttgctctatactttttaaaattttgcagttaaataatcttgtttttgaaaacttggctttaaaggaacacttgacatcccagttttcccttccctcttctctaccttgtgtctgttttcataagagcgggttgtattttacctcagatttctcaggagaccagaactgatctgccagccttgctgccttttgttgctctgctctttacattgtctttacagtcaccaggtgtcaagatgctgcaagtacataggactggactgggcaggctgggggtcagcctctccaagggtcttcaccacaaagctgtgttggccgtccggagggaggatgtgaacgcctgggagagaagggccccgctagctcccaagcacatcaaaggcatcaccaatctgggatacaaggtcttgatacagccttcgaatcggcgggccattcatgataaggtgagtatttccaatatttttatgtgagaataaaatgaacctgcatatgaaaattaaggagcatctgagtgaaaataaaatgtttgctagtgtcactttgatttctttactttgattaaaagtattattttcaccctatgcttactccagctgaggaataaaagaacttacttttaggagatgttatttttcaatcattctctccttcatgtacagccctggttctgggactagggggataagactccattcttgcacccagagaatgctcctttgagaatgtaccttgcctgccactctgagagaagcctgtcatgttcatcattccacttgctcttcaaaataatattatggtataggtttgactcattcctattttagcaatatacatatatatgtatacttttcctttcctgagccattttaaagtagatcacagacatcataactcattactattaaatgctccaatgtgcatctcctcagaataagccatgttttctacataaccacaatatgactttcatacctaagaaattaataattcataatatcatcttatatagtctatgttcagattccccattgcctcaaatgccttttattttaattgaggcttaatgaagctttattgcattttattattgtgtgtctttagttctttcaatctagaacagcacatccctttctgattttgtttcaatgatattgacattgacatggactctagaccccaagacttacagagtgtcctgctactgggcacgaataatctggttgttttctcgtgaatagattatattctaattatattctatatgttatattcatgccaaaaatgtttggcatgaatacaacataagtgatatatgttcttcccatggcatcacatcaaaggcacaaaacatctggtcgtcccatggttggcaatgtgtggtcacttggttgaggtggttacagtgagagctcttcattgttaaagtctatatcaccctttgtaattactgagtcatctgtggtatggtattttaagatctgtgatgttcctattcctcaaataactttacaactaatcatgtcagcctccattgataatctttgcctggatcagtttttacatggagacttgtgaagtgatgcttttctaattttatcactcccctaagtttgtttgctgacattcttctttagaaaagaggttttcttttctttctcttccactcctccacttttttgagttgtgaactgttctattacagtgcattgaactgtttaccaaactgtgaattctgtaattcagtgtttaaacaatttctttaaaaaactttcttatcattgcatttaaagccttgttatgaggagtttatatatacatcttcatgttgacaagatgaatcttaggatgttgcttaaaagatgagttgtgaactgttttattacagtgaactgaattgtttatcaaagtgtggactcaaatttgctattgcattatgcttctttaatttttgcttttttgagtatattttggaagcatggacctttttcattcaatccatttaccattattctttttaatgtctgaattatcccaaaattggttagtgtgagtccttccaagggggcttctctgtcctttggatgtgaatccatttacctttgagctttcttgtttcttaggataacaaattgtcctagtctcaccttgtacctttcctgccccacatcaggaaattaataatttcaccaagaagacttgttttcttttattatatagagaccaagatctggtatctgatgttaagcatgtttattgttactgagatactgttgctcttaggctctattagtgtacagagtttagaaatatacacaaacagttgcccacacatatatatatatattatttaatcatgagttaatactgatactaaaagacctccagaggaaaaattaatgtccaggaaaattaaaatttaacattgcacaactttttacctttaaaattttatatttgcatctgttttctccaacagtaaaaatcttaattctttaatattaatgcatattgtttgctttatcctatgatatgtataaatagtgtcaggatcacaatactattattacaactaatagtaagcccactaaatgaagtttactatttctttgattttttttttttgcctttagaatatatctgacaatgaatattcagagtgttgtattaaaaaacttatttgaattttttttggtgtggtttcgtcatcaatctaatatccatctggatttatatattattgtttctattcagttttaatgtttgctttcccatttttgatttaattttgtttttcaaagtagaacatttacaaattccagaaatgaagtcatgttttcattttaatcccctacatccaattctcacccaccccaaagaggttacttttgtttctgatctatcattgttttttcttttttttttttttgcaaaaataaacacctgtgtgtgtatttacatatttatttcccttttgttcttatacagaaggctgtctactatatacactcttttaaccttgctttctttctttagtaacaacatatcttgaacattgctctatgtaagttcttagagagatttcatttcatttcatggctgtgtgataagtactccattgtacagatttaccatggtttatttaccaatctactacaaatgagaaaatggactgtttttgaagaaaccagggagaaattgtatattcatcctttaactatctctgttattcatctctactttcctttgaggtagattcctaactaaacttatcttttgttcaattgctcttttaattctgcttgggttatacctctttatttaaaaatcagatatgtctgtctgttgcttccatgtactagttatgcaatctcaattaagtttgtaagcctcaggtttcccattgattgaatgaggattataattgtacttacctcatgagagttgtatgaaatttaaatgagataattcatgcaaagagcttagcaaagtacatgtaatttattaatacatatcaatgattgttacttattacccaataagtcttgtattagccatgatgacattcaacctacatttccaaatcggtttactcgtatgttgtaaatgtctctttacttctctctttgtatttttatatatgccttcaatgcctcttactgatattaccatcttgtgtttgccatcttcatttctctaaatttcaggtagcctttcagattaatttaaattctcatatttctttggtatcctacctttcagattaatttaattttcatatttttttgatcatcctacttaggaattatctcaatatcagttaaagtagcatatcacttagatactgctgatcatgttttgtatttttcatttactccaatccagagtactttttactatattttgtgagctcctttatacaagggatcttgacaatctttccttgtatcttagcatctcatctctagcacggactaatgtacagtaaggatagtaataaatactaactgggtaaacactgaaaaataaatgtataattatgtcttatcttttaaaaatacaactaataaatcacaatttggcatttaatatttcagtgtactgttggtacaaaatatacatttcacaagtatgcttaagtcacatgtgctagtgcatatgaccaaattataaactcttcttaaacatacatatatatatatatgctgtgttttctaattgtttaatgctgtaagagttgacatgatagatttatctttagccaacaagtacaaacattacaagtagttgttctattatttgttctaattcatacagattttattccttcaaaagatttgttgtgcctttgctacacattttgctcaagaaaacatggatattacaaatcaggataataacctttaatcataattacaatttcaggactatgtcaaagctggtggcattcttcaggaggatatttctgaagcttgtctaattttaggagttaaaagacctccagaggaaaaattaatgtccaggaagacttatgcatttttctcccacacaataaaagctcaggaggccaatatgggcttgttggatgagattctaaaacaggtaattagtgattaatatttataaatattaatgtagaaaaataatattttttacttttaacccccaaatggagttttagtcttcttttcttctttagtttaagagaaacattgcattccttttatattctgaaatggaaagaatgataaaagctcttaccctaaacaatttaccaatttataaggtcaattggccatatactatcaaatagaattttagaactgaaaataacctcagggatcatataatctactaccttgtttttcagatgggatttttttttgagatggagtcttgctctgtcatccaggctggagtgcagtggcacaatcttggctcactgcaacctccgcctcccaggttcaggtgattctcctgcctcagcctcctgaggagctgggactacaggcgccagccaacacgcccagctaattttttgtatttttagtagagatggggtttcactgtgttagccaggatcgtctcaatctcctgacctcgtgatccgcccacctcagccttccaaagtgctgggattacaggcatgagccaccgtgcctggctgagacaatttttacatttattcacattcataagggaaaatgcatttttgagatagaaatactatgaaaaaaactagcagctgtttaaaacacaagatattcatttaacaaatatctattcagtgctgatgctgtgtctgacactgtttgttccaaggaaagagccaaagagtaaagtatctttattttcacaaagcttacattctagtgggaggagacagataatcaatgaataagacatctaatatccacttgggaatatgtgtgatgaagataaatataatattgaaggagaaaaggagtcctgggcactgggaggtgatgatgctattttaagtaggatggtcagggaggaccccattgtgaaagtggcattgagcaacaatctaaaggaggtgaaggaccgagccaatacatctgaaatgagtcttttaagcagacagattcgagattgagtgtggggttagagtacaccatgttcagaaatgcttattttgtctcttgtgtcaacaatggttggaggtgggaggaggagagcaacattactaagaaggctgctaacctaggctggtctagactgcaagtcacaagtgatggtggcctgcacctggaagaaatggtggaagagaaagttgtgaaatgtggacaaatcttggatatgttttagaggtggatcctacaggattcactgatgacataaaggtggaatatgaaaaaaggagagcagtcaggaatgactccaggttttggcctgagctacttgaagcaattgtcatggaatgacatgatgccattacaagagaaaaaggcttcggtgggcaaatcaagagttgagtttagggtctgccaagtttaatattctattagacattcaaagggataatctaagagggctgttggatatgagtctgaagctcaggagggcggcttctcctgacatacattttgtagttgtgagagaccaggtggtgtctagagccacgagactggatgagcctaggaagggagtatagaaaaagaagtctaaggtccacatcttccagatgttagagctaggatccgagattagggcagaggattggagggttcataaacagtaaaagcaatggaaaatacctattatataggagtgtaaaatacttccaattgattccaaatacagtagaccagggtgacagttaatttgtagtatttctaaagaataagttcaaaagtaattaatggaaataatactataggaagaatgttccactttaagcaaataaagtaattataaattactttaggaactttttttgttattttaacttttgttcattataagaaaattaaaaaaaactgacctaacagggctgcttttgggcagcattttgtaaccaattacttggagttggaagaatgataaacattcttaaattcatgaacataataaaaattatatgatctttgaggaaataaaattactgtttaaaatagtgtataattacaatacactaatatgttttaagcatgaattactaatgctttcctgagagaggaaaggttttcaaggaaagaggaagaatatgtttttagtgaaggcaaggaagataaatgacataaaggtggaatatgagaaaaggagaggagtcaggaatcactccaggttttatcttgagctactcgaaggaatcctgtactacttttaataggaaggttacctgtaaaaggaaggagagggaaaaagaaaaagagaggagaaggaggaggagagagaaaaatatctgtgcttggacttgagagagagtgttatttcccaggttgaaagagagtgttattttccaggtagaagagacctgagtgtacttagaatggttgagaaagaatgtaaagatggatgatgcaagaaagaggtaatagttcaagaagagtggtcttggaggacatgagagaagctgggatctaggtcacagagattcgtctcaaaatggatctgttttatttgaccagaaggaaggatgtaaggatgagaggggctttagacatgtttatagatggtgctgggtgggtcggggcagaaaggtaaaggaaattctacctttattatttcattgcttaccatcaaggggggtatagcacctgctacaaacattttttcatttctaattacaagcctaccattggtttgctacctggcgttttctaaatatttcatggtatacctgatggtagcttctgttagcaagttaaggtgtatttcagaggaaaaaatgtgaaatatttacttgctcatatatgtctttacttgttaaaattaagcttaatgggtccagttgaaatttttttatcctcttttcaggaaattcgccttattgattatgagaaaatggtggatcatagaggagtacgggtagtggcatttggacagtgggctggtgtggcaggtaagtatgccagggcttacatcacccacttcctaaattggcttggggaataactgtgagttttctcttttctgcggcaaggggataggagtgtttatttccatggaaaaaattaaaatgaaggtatgctgaaggatgatgaatacaagtggaataaagtatgaagataaatggttaaaaatattttattgcaaaaattcaaaggaaaaagcagggagaaggataacaactacaaacaagctctgatttttaggtagttcctttcgaaaggctggaaggagataccagttcggtaatgagacattgagaattagaagatagggagaaatagaaagggaaaaaaggtccaggggcaggaattttgtccagttaattctttgaaccttgtttggttaagaaacaccacaatatgttgtagctctgtttcagatcagactacttgcaggtctaataaggaaggtcatgacttcggtactatttgtttgaccttgttatataaaaatagaagattccttatgtaaaaaataaaagtagaggttcctcttcaaagactttcgtccccatttaattaggaataaatagtaacttctcttaggagcaaaatttattcagacctgtgcttctaaatatttgccccggcaggcttatactggtccaagcaagcattaagtcatagcctgttcctcttccttatttaaaagtgtttttacctttctcagcattccacaagttacttcctccttcctttgttctcctctacctttgcctcttttaaaaagttctaagttgctagccaataggaacgaatacagaacgtgaggtctcgttccagccaatggaaaccggacacagcagtagggtggacgtgtcaggttataaatgaccctgtctcttttgttcggtgtattcttgtggcaaaactgctggtgagtgtaccctttctgcaggaagtaaaaatggccttactaaataaattaaatttatgttcatgtgctgtttctttacggcaccagggaacaagcatttcaaacacttattatggcttatgtcattctgcttattattaatattatacttgcatccacctgctttttaaataataaatctaagcaaaaatgtgaaattcaaaaaattcttttacttaaaaccaattcaggcagggtatggtggctcatgcctgtaatcccagcactttgggaggctgaggcgggcggatcatttgaggtcaggagtttgagaccagcctgaccaatatggtgaaatcctgtctctactaaaaatacaaaaattagccgttggtggtgcatgtctataatcccagctactcagaaggttgaggcatgagaattgcttgaacccaggaggcagagtttgcagtgagccgagatcacgccactgcactccagtttaggcaacagagcaaaactctgtctttaaaaagaaacaaacaaattcattgtttttattcactcacctcatatctttcctcacccaacacccaaatcagctctattaaactaacagaaaattaaatttcttagtataaaagcagtaatagagagacctctgatgactatgattcctagggcctttcaggactcatccagatgagggttttatttacctagacagaattaatacattggaattaagttgatttatttagaagtcaccatattcagaagtaggaccacatccatgaataggatgtggttcatgttcttagagaagcattcaagcaagtaaacacaaatacaatgctagtgctaattgctataagaaaggtatttgagactgggtgcggtggctcacacctgtaatcccagcacttcgggaggccgaagagggcagatcacctgaggtccagagttcaagaccagcctggccaacatggtgaaaccccttctctactaaaaatacaaaaattagccgggcgtgatggtgggtgcctgtaatcccagctacttgggaggctaaagcaggagaattgcttgaatctgagaggtggaggttgcaggaagccgagatcatgccattgcactccagcctgggagacagaataagactccatctcaaaaaacaaacaaacaaacacacaaaaaacaaacaaaacaaaaggtatttgaacttggctgttgcaaaagctttcttttaaaaagtgcctttgaaggtgaggtctgaagaatgattaaaaatgattcagggaagtaggagaggacagtacaagtgtgcaaagctcaaagtgaagagggggaaaataatagttttgaggaatagaaataagttcagtataattagagtgtatatggaaataactatatgtggttcttataagtatttagaaggtggaattcacaggatgtggtgactgattggatgtaggagtgagggggtgagggagacactttaaagatgacatgcaggttaccaacttggacagtgattcagttctttcagtacccacaggaatggaggaagaatttggaggaagggagtaaatgagttctgtataggacaatgtattagtagattacatactaagttgctataatggagactccacaaaacagagcttacataagctagaagtccaggagtgagaagtctagggatggtggaatgagaggaagcctttgctattgtcaacacttggctttcatttctaaggtgacccttcaggttgctattttccagtcagtggaaaggggagaaaggaagtaaggtaaatagcttcctttctaaggatgtgaccaagaatctgaacatattatttctgctcatacccattggtcaaaattgactgagaaatgtaatctataactgggcaactatgtctgctgcaaaaaccatgtgccctccatctgtgaaagattaactgaggtcagacttactatcctgtcattaaccaactagaaaaattgaatgagatatagaaaacaatgttatcagacattggaccacaggcagtacagacttgtgatccttgatagatggaaaatagatgaggtgagctctatgttcaccctggctttctgtctggagaaattatcagatggtatgaatatggaaatgtgacacaaagcttggtgaactcaataagctgaggagataggagttggggagcctgagttggctggaattggcaacacatagtaccaaacagtgaggagctatgcagaaatggtgttccagaaatatgcttaggggtcttgctaagtggaggatgaaaattcatgagactgccttaggaacaatttgtggaaaagaacaattgccagggaactctatggaaaactgttctcagagtttgtcccatcagccagagtggagagacttaactgaatgtataaggctttcagggatgccctgaagtgtgtggctaaaccagctcttaataaagcctactctacattcattcctataaaagcttaaaaatgagcctaatgtctactttgagagagatgcaattagcacacaagcagattaaaacagctataataaatgttcagtatgttcacgttttaaatggaaacataaacaaaatcaggagagaaatagaggaaatcagtccatctacctatcaatcaatagataccacggacgaaaatctatacataagcagaggattagagaacatatatagattttcttctgtagtgtctattgattgatagatacatggtggatagattgatttcctctactcctcattttgtttgtgttttcatttaatccttgagcacattgaccagcatctctgtcatttcttgtttctgggattgtttccgttgttgattatctccaggttaatgttccattgcaagactcataattttttactgaatgccagacagtgttcattttacattgttgatagattttgttgttttccttttaggagcattgggctttgttctaataggcaattgtttgtggattacattgattctcttaaggcttgttttcctttcacctgcctctcttccttctgggactccaaatacacacgtattagagcatttgttcaccacctgtcacttaaattctgtttttttttctcagtctttttctctctttatgctccattttgtttaatttctactgcagtgaggaaggaagaaagtaaagaaatcatgaccggaacattcccatatcttgataaaaattataaatacacagataagtgaagttcaataaaccccaagccaaaaatagaaataaaaaatcacaaagaaaatgacagtagaaccatcataattgtcttgctaaaaaataatgataaaaacattgaaaataaatgggaaaagggaaccatatacatagaataactacgataaacatcaaacttctaatcagaaacagtttaagctagcagaaagtggagcaacatctttaaagtgctgaaagaaaacaaacaacttataattttatgcacagtacaatatatttccaaagacaatatcttttcaatatctttctggacatctttctagaaaatgaagattaaacacagattttctggtcaaatgaaagctgataaaattcactgacatagaagtatttaaaaaatttctcaagcagaaggaaatgaagtcagttgaaaacatggattgaattaaaggaataaagaacactacaaatggtagaatgtaaacaaacataaaaaacatcatatcttattttaaaacttttctttagggctgggcccagtcgctcactttgggagtccagggcaggctgattgcttgagctcaggagtttgagaccagcctgggcacacgatgaagtcccatctctacaaaaaaatgcaagagtagctgggcttggcggtgcgtgcctgtcatcccagctgcttgggaggctgaggtgggaggattgcttgagtctgggaggttgaggctgcagtgaactgtgttcatgccactacactccagcctgggtgacaaagtgagttaactgtctcaaaaaacaaaaaaattctttaaaagataattgatagtttcaaatgaaacaatgtattgtgatattcataacacaggtagaaataaaacttatgtcaccaataatgatggacaggtgaaaaggaatagaaccatacttttgtaacaagtttattttattataccggaagtagtataatattacttgatagttgactataataagataaagatgcatatggtaacctagaacaatgattaaaaataaaacaaggctctaaagctaataagccaataatggatgtaaaattcatactaagaaatacacagttcaagcaaaaatacaagaaaaagggcattaaaaaaacaaagtacaaatgaaataagaacacaaacagcaacatggtagctttaaatttagctatatcaatagttacattaatggcctaaaaactcccagataggctatagaatgagtcacaaaacaatttttgataaatttgaaagtctgaaatcatacagtatgtattctgactgtaatagaattgacctagaagtttttatagaaagatatttggaaattttccaaatatttgggagatattcaatgtaattttgaataactgatggattaagaagaaatcacaggaaaattagaagatatttttaagagaataaaaatttaaagtatagcttatcaaaatgtgtggggtataaagtagcaattagaaagaaatttacagcatttaattcttttattagagaagacagtttttaaattaataatttgtgcttccactataagaagctagaaaatcaaagagaaattaaatcaaacgcaagtagaaagaaataaataataaagatacagagaaaattaatcaaactaaaacaggaaaagaatagagaaaatcaatgaaaccaaaacctggttctctgaaaaatatcaataaaattaattaacatctaggtagactgatagaaaaagagagaagacaatatttactaagaaggaatactagggggccatcactatatcttttaaatattaaaaggataaaggaatattatgaataggtcaatgaatttggcaaccttgatgaactagataaattttcaaaatgtacaaattaccaaaatcagctcaagaagaaattgaaaacttaagtatcaaggtacaaactaaattccactaataggcatttattaagagaaatacaatattttatacaaagaatttttttatagcaactccatttataatagcctggaaacaacccaaatgatgagcagagaaatcaactgaaatagaaatgggaaactcttcaacaataaaagactatactgatacacacaaaatatgtatgaatctcaaaaacacgatgctgagtaaaagccgacacattgtaaaagagtacaattgacccttgaacaacacgagtttgaactgcatgagtccgcttatattcatgggatgcaaaatccacctttatcgggggccaccttttcatacaggcaggtgtcacagaactgactgtgggacttgagtatgcccagatattaacatgtgtaggggtcttagaacgaaccccctgcatatactgagagacgactgcttaaaattttgtttatatgtaactttagaagaacttaatctatagagagagaaagatcagtagttgtggagaccaggagttggtgaaatactctgtatcttgatcatggcggtaataacacagatgtgtatatttgttaacagttaccaaactatacattttaaatgggtgcattatgttgtatgtaaactatttctttataagttgactttagaaaaacgacaaaagaaaatttggtagggatgtttgttgggggtgaggatggggaatggaggagcatggaaagttctttcctaaaaagaataaggaaagaatggatgctcaggtctttgctgcagggatgctgaattttagctttctgaagaacatccaaattgagacagacataaaaaggtggctggctaagttttgaaatcaaaagaaagatctgttttcaagagagagacttgaagtcgtctgcatatagatagtacttgaaatttaggactagatgagattacctaagggacaaatgtagatatggaagaaaatagagcccaggacataatcctgagggtgaataatataaaaggaattgccacaggaagagggtcctacagaaacgtaggattaaaaatttcaagttgggatgctgaggggcactcttcgtttcaacaaagaagaggtctgcattgccaagtacttctgatctgatctacaatgtgttcactgagtttagctatttaggtcactaaatagatgatgggagccctagggttaatggtgactttgtttttgtttgttcatttgtttttgttatgggaggcatgggagcatgtttaaatgctgataagaatgaagcaatagggaaggatagtgaggggagtaactcagtaataaattccttgggtgacaggaagggatgggatccggagccaaggtggagagattagccttagataagaagagaaacattcttcctattttatcaggattgaaggtggaaggaataggtggggatgcatttgggttagtgcatttgatggcagaaagttgagaggatggatacaatctactgaggcctcttctaaatctgagatgcatgatctgtgcttctgcagggcaccatgcagattggagaacgagcagagagaacagcttaaattcttcccaactaatagtctttctattggtctgattctgtaacaggaatgatcaacattttacatggaatgggtttaaggctccttgctttgggacatcacacaccttttatggtaagagatgtttattttgctaatttttttgaaaacacatctcacaccttaataataaaaaaaatgctgagctggtggactaacttgagttgtgtatgtttgttttcagcacattggcatggctcataactacaggaatagcagtcaggctgtgcaagctgtccgtgatgctggctatgaaatatctttgggtttgatgcctaagtcaataggacccttaacatttgtgttcacaggaactggtaatgtttctaaggtaacttttacctgacttccttttgtaaaacaatccaaaactttgaaactaacccaaatgatgtgggcagccattttgtgggtatttcaaatggactggagattctattttaggcacttttcttaccaatgtatttttctcatagtgaaaagtagtaatgatctaaatatgtgtgtatgtgtgtatgtgtatgtgtgtgtatatatatgtgtgtgtgtatgtgtaagtgtatatgtgtgtgtatatctatacacatacatacacacatatagcttttagtttattgcaagctatcggagtcttggaatactttttcttcgtttctttttctttttttaaaagagataattcagccagacatggtggctcacacctataatcccagtgctttgggaggccgaggcaggcggatcacctgaggtcaggagttcgaggccaacctggccaatatggtaaaacaccacctataccaaaaatacaaaaattagccaggcatggtgacgcacacccataatcccggctactaaggaggctgaagcaggagaatcgcttgaacccgggaggcaaatgttgcagtgagccgagattgcaccactgcactccagcctgggtgacagagcgagactctgtctcaaaataaaataaataaataaataaataaataaatagataaataaataaataagataattcattgttaacagtgtaaaggtctgttctgagggtgatctgttttacctccatcaccaaaatgtccacgagtttgaaagtgacctcacgataaccattttttaagactacttggagttttgcaaacaagatgttaggaagattcatttaagactggaattcatgctttcttccttctctggtggttttaagaatgtgtttgtaatctcctatagaaacttgtcattgtaaaataattcctgcttcaaagaaaatattttacaaataggatatcattcttattatattgtgaggtaagcatctgattatttcttttgtaggaaatataatttccccatttgaaataatttctgcaatggatctaaaataaatggaaatatttttccctcatttctaaacactcttatcgatactctgagtagcttaagtaaaaaagaaaaaaattaaagccctttctaagtgtgctgttctaacaggaataaaagctagctagcaggctggttgtaagcaactcatctgtatttctgtgttgcagtcctggttgtagggaaggcaagtggagctatgtaattgttgggaaaatgatcagatctttgcagaaaaccatgttggttctttttctattgatttttggatttttactcatgtgttatttgtagggagcccaagcaatctttaatgagctaccttgtgaatatgtggagccccatgaattaaaagaagtttcccaaactggaggtaagggacaggagatgactaacttttaatgtttttaacagtctatgttataatattttatcaccataaatttttattatttgtcacccactttactaatttctttctccttttcaagacctcagaaaagtgtatgggacggtgttaagtcgtcatcatcatcttgtcaggaaaacagatgctgtgtatgatcctgcagagtatgacaaacatccggagcgctacataagtcgttttaatactgatgtgagtatcatattcaccttttaagttggttgcttatgatacagtgtaggtaggctattttcaaggagagaaattatgaattgtacaatggctttcagttttcctgttgtgagtcctttgggtatcatgatttggcctttggagctatcatcttttatggtatttggagtagtctcgcttagtgcttttggtggtttctgtggatacactctttgtttcactgtctccatttctgcttcaggtctacatgtgcccaatagcgctaagccatttcctgtgcgttactgttttctgtatttatcctggactagttagcactgtgggcagagcataatgcctatgacgtttggtctcaacatggccgcaaattgcaaccataatctcagccagcaatcttgaaatgcatgccattgattatcagggtgagggttaaatagcgttcctatataagtgcagaccatccccactgaatgaaaaagctattacacttacaaatcaaatccttcgatgcgacacacatcttataagaaataaaatatatttgtgtgctctatagtcattggccatgcaacattcttcccccagataaactaacaaaccaaaaaatacataaaacaaaaaatgcatgcatttattgtgaaaaagaatgctttaaataattttaaatatgtttgaccaacataacattttcagattctggtatgaagtttagcccttatatcgtgcagggcatgttagggaaaagaattcttaccttcagcttgagatgtttgaagaagaaaaattatgaacatagtttcaaactgaaaaatttggaatctaagaaatctttattccttctcatttccccaatcacagaagtattggtgattggatgcatcctaaaactgatactgacctagttgccatgtatgcatgtatgtagcacattgctccatagagcttacagatgttatccgatttaatcttcacagcattcctttgaggaatggggtgcgtattatccccatctggcaagtaagaaaaatagagcccagggaagttgaggtagcatgatttaggtcataccctactgatattagagctggaaatataattttaggtccaccgttttttgtttgttttgtttttgtcctttcatttttcttcggcatgcaatacattatagaatgaacacactaatgtatttatagtcatatatttctctatcatagggtaattctatactgaaatctgggtggctatttaatatctttgcctcaaaagatggacactgacatggacaataagaaaatcaattagaagttacatttaattatttctcatagaatttactgatagctttctttcagtatactgtattaaaataggctattttctattttcttgaacctggaacttgcattacagattgcaccctatacaacttgcttaattaatggaatctactgggaacaaaacactcctcgcctcctaacccgccaagatgctcagagtctcctggctccgggcaagttctcacctgctggtgtggaaggctgccctgcattaccacacaagtaaggactctacttcagcaacgatagtagttttgaccagctgcttccattattagaggatcaaatatcacatatcagaggacttgatttctaatgactacttctctgccttatctttcttgacctcttggcagtactcaacactgttaaccatctctccttttttttttaaaattttattgtatatatttatctttagagatggggtcttgttatgtttcccaggctggtctaaatctctggaccccaagcagacctcctgccttggcttcccaaggtactgggattataggcatgagccaccatgcccagcccacctccacttcttgaaacattcttcttttgtcttctgtgatgccccatgcagctctgaagctatttgctcagagaagtcttctcttgtttctccattctcatctctatctctagtctgttactctgttttttgttttgaaattcctcatttattttctcgtgtttatgatttttctccctcttcctagaacttaatctccttgaggatagggggtctctttctcttggccacaagcacctagtacgttgcctgatatataataggcattgaaataattattaaatattgaattaattaattaatatttgtggaagagtaaaagatatgtctgcttttctaggcatgtaactggcatcaagtaaatgccaagggaatgctaggagcaacaacatcaccagggtttagaagaaagcagaagcattgggcgcagcaaaccttaaagggaggaatgaggagaacaagaattccaagtgaaaggattggtgcttaataaatatggagctaccaaagcatatttagggtgctgaatgtgtcagtctagctctacattggttcatgtatgcaagtggtaattggagattgggcagaaaaattgtagtcaactttttgaatgctggactgaaacttttgttcgtaattctagagatttatgtgtaaccacatgtaaaactatacagtactaagcagggtggaggtagcataattgaacctttctgagttatttagagataactatgttaactttagtctgttaatgatttctctaggaaagcaattttctgcgtttgtttccttttttcttttctttctttctttctttttttttttttttttggagactaaaattcatactgggaagattctctgtatcacagaacagtgcagagtcctgaagaatgagctataggaaaaagaccccaaaagcctggaaaatatttgggatccacattgaaaattgttccaaactcctttttttttagtcttgttggagacttcagaatttcatcctctcatctaagtattgatgttattttcagactcgtggcaatatgtgacatttcagctgacacaggagggtctatagagtttatgactgagtgtacaacaatagagcatcccttttgcatgtatgatgcagaccagcatattattcatgacaggtgagtttgctaaagttgttagatgatattcctcacttagttacattgatatactactctaaaattctcagtattttacatctttatgaagttatgaaaatttggagagatatactcaaacatactaaaatttggggaaaaaaacatgcacatttgaacacacacacatacacaaatatatttaaatgtgagatgggttgctgaagaaggaaaacgttttgtttgaaaagttttgtttattttcgcgtatacttttatacaaaattacaaatgataatgaagcaaatatagtggagtagacatctggaaaagtcagaacttacaaataatgttttgtgcttctgaataaaccacattgctgcttttagtgttgaaggctcggggatcctgatgtgttccattgacaatttgccggcacagctcccaattgaagctacagaatgctttggagacatgctttacccttatgttgaagaaatggtaagcagactggtaatatcagtaacaactctgtggctttaaattgaataattgagtaattctgtgaatgttctggtcttctgtatctaagaaatctgctgtaaaatgaaatgccagcccttccctgaaatacttactgatactattttatttgacatattctgcttacttgtctcttggggtataggatgatatcactgccttcatgcttaattacaattacatctctttcccattataggtcttttattaagaatgttcaccacgtgtgagataaaacatgctttatttttaattttttgccatctatgtcagttcatattggggcactcttattgtgccgtaataaccaggtctgaaaaattagaaatatagaacatttttattgggttctggtcactaatagttaagtgacagcttatgttgaggtcatgtatttttcttctgtgatcattgattagctaatgaaataccatgttatctaaattccattttctttatcaaagaaaatttttcttatttcagtttttatcctaagcattcatagccttcctgctttatttttatttcagactgtctgcctctgccaatgattccaatataaagaatggctcttactcttttcatctatatcttccctcataagctcttacactaaaatagaacaatagttagtggggcaagaataaatgaaatttccagtctgtgatcctcttttctttcaaatagtattccattttgaccctttctttttcctattggaatatgtcaacagggagatgtagcaggcttgagaaaatgcaggcagcaggcactgtcatggttagacatgatattgcacctactaggtgcaggaagtgttcaaagtactaaatatattttaattttaagtactcatgaaaaccgtattaggtaaataccatcattaaatccattttaccatcattaaatccattataaaacaaatacagagaagttaaggacgtgttgctgatacagtcagtgctggatctgcaatgtgtctggagttggtgctgaggcattaaagaagcaggagaaggagagtacaaatgccaggctttgctggagatatcaacttgctttataaccttgagcagggcactcacttcctttctgagcctcagatatttcatctgtaacatgaggtggttgtaatgaatacataatcttattttattttattttatttttttgagacagattctcgccctgttgcccaggctggagtgcagtgatgcaattgcggctcactgcaacctccacctcccaggttcaagcgattcttcctgcctcagcctcccgagtagccaggaccacaggtgtgtgccaccacgcctggctaatttttgtagtttttagtagagatggggtttcaccatgttggccaggctggtcttgaactcctgacctcgtgatccacccgcctcggcctcccaaagtgctggtattacaggcgtgagccaccgcgcccagccacgaatacataatcttaacagtttctctacttctaaaattttataatggtaaatgtttaagtggccaaactgtatgtatataaaaaatacaataatatatttaccaaggtgtttaccatttttgatgttcttcattcatttccctttggcatcatttccctgcagactgaataacttaatttatcacttcttacagtacaggtttgctgatgacagattctcttagttgtcctttatctgaaaatgtcttcatattctctattctgctaatcagacatttctgtgcatttttaatttccgatgttctattttcagttctagaatttccattgggttcttttttaaaaattttgttttggtattttctctttttctgcagagagtttctatcttttcattaattatgagtgtatttttcgttattgcatggaacatagttataaaaaccactttacaatgttcatctcatacttccaactttctgggtcaatttgtggttgaactttattgattgtcttttcctttgagaattggtcatagtcttctggttttcatagactgaataattctggattatggcctggatgatgtggatattatgtacatgtatcaaatgcattttctgtattcattgagaagatcattttttccttttctctgttaatatgtgaattgtgtcaattggttttcaaatatcaaactaacttcacagtcttaggataaatccaacttggttgtatgttaaccttttatttatgactaagttctggttgctaagaaatataccaacatttattaatctattttaccattggtagacatttggggttgtggagatatatatatatatatatctccacattattttgttgagtttgcacatagatttatcttgtttaggattattaacatctatgttcattagagagcttaatctataactttattttattataaggtctttgtcaggtttggatattaagtttatgttggcttgaaaacaagttagagagtatttccttatttttggtctcaggaacattttacatgagattagtgttagttctacttgatacaattcattagcaaaactatttgggttggtagcatttttgtttgtttctttttttagttttaagggtaagttttaaaattatagattacattttatttaaaaacttaagaatgttcagatttttatttttctttgtcaattttggtatgcttattttctaggaatttatatttcatctaagttgtaaactttattgtcattttttttttagtaataaccttattaactctttaatagattctgtagtgatttctctttttcctgatgttggttgggtatgccttctgttttttcttaatctgttttacctggacttgatcaattttattagtcatcgcaaaaaatcagttcaactttggtgatcatctctattttatatcattttctgttttcttaatttataatcttacctatactatattctttattttactttattgagagcttagttaacctttcttttcataacttcttgcaataagtgcttaggtaattgttttcctgtgtttctctttagcaatatgtgcattttaaagccattagatttactttgagtacagcccatttttgattgtattatctcttgttttgttctttgactttcttgtatattctgagtattacttccttgtgagacgtatagtttgcaaatactttttcccattctgttgtttcttcactcaattgatcttttgtttgtttgttttgctgtgcagaagctttttagtttgatgtaattccatttgtctgtttttgggttttgtgcttgtgcttttgagttcttatccaaaaaattcttaaccaggtcaatgtaatgaagcatttcccctgtgttttttttttcttctagtagtttcatagttttaggtcttccatttaagtgtttaatccattttgagttgatttgtttatatgatgagaaatagggggtctagttttattcttctgaatgtagatatccaattttcccagcaccatttattgaagagactcttcttcccttaatgtttgttcttgacacttttctccaaaactggtggatatagatgcatggatttctttctgggcttgcttttctgttccattggtttatgtgtctgctgttatgccagtaccatgctgttttggttactatagctttgtagtatattttgaagtcaggtagtgtgatgcctccagcttcgttctttttgctaaatattgtgtgacgatttggggtcttttgtgatttcatactagttttaggattgtcttttctatttctgtgaagattatcattttttttttttttttggtaaggattgccctgaattggtagattgctttgggtagtatgggcattttaacaatataaattcttccattccatgaacatgggaatatctttacatttgtttttgttttcttcaatgtctttcatcaatgttttacagtttttagtatagagatctttcatttccttggttaaatttattcttatgcatcttatttttgctgtggctattggaaatgggattttttttcagattgttcacagttggcatatagaaatgctactaatttttgtatgttgattttgcattctgcaattttactaaatttatcagttccaacagtgttttgatggagtctttagggttttctatatataagatttcgtcatcttcaaacaaggacaatttgacttcctgctttccaatttgggtacctttgttttctttttttttgcctagttacactgactagaacatccagtactatgttgaatagtagcatccttttcttgttccatatcttagagaaaaagctttcaattttccatgatcagtatgatgttagctgtgggtctgtcaaatatggcttttattatgttgaggtatgtaccctgtatgccaaatttgatgaaagtttttatcataaggtgatgttgaatttgtcaaatatttttttctgtttctgttggaataatcatatggtttttgtccttcattctgtcaatgtgataaattatgtttattaatttgcatatgttgaaccattcttgcacccctggaatgaatcccatttattatggtgaatgctcttgtaaatatgctgttaaatttggttttctagtattttgttgaaaatttttgtatctatgttcttcagggatattggcctgaagtttactttttttgttacgtccttgtctgattttggtatcacaataacactggccttatagaattagtttggaagaattccctcctccttgatattctggaatagtttgagaagaattggtattaaatcttctttaaatgtttggtagagttcagtagtgaaaccatcaggccctgggctagtctttgatgggagactttttattactgattcaatcttgctgcttgttatcgattcgcctattcagattgtttatttttttctggttcaatcttggtaggttgtatgtgtccaggaatttgttcatttcttctaggttttccaagtttttggcatatcattgttcataatgctctcttatgatcctttgtatttctgtggtataagttgtaatgtctcctttttcatctgtgtttttttttttttttttgagttgtgggtgatttgttatgcaataatgttgcagaagctaatacatacttaaaaaaaacttttaggttcaggggtacatgtgcaggtttgttctataggtaaacttgtgtcacgaggggtggatgtgcagattatttcatcaccagggtattaagcctactacccaatagtcattttttgtgctcctctcccttctccctcaggaaggccccagtgtgtgttgttcccctctatgtgtctatgtgtcctcatcatttagctcctgcttataagggagagcatgcagtatttggttttctgttcctgcattaatttgctaaggataatggcttctagctctatccatgttcctgcaaaagacatgatctcattcttttttatggcttcatagtattccatggtgtaccacatgttctttatacagtctatcatttataggcatttaggttgagtccatgtctctgctatttacaatagcgctccaatgagcactctttacaacagcatgtgcatgtgtatgtgtccttatgatagaacattttatattcctttgggtatattacccagtaatggattgctgggttgaagagtagttctgtttttagctctttgaggaattgccacactgctttccacaatggttgaactgatttttactcccaccaacagtgtataagcattcgtttttctccacaacctcaccagcatctgttatgttttgaccttttagtaatatccattctgactagtgtgagacggtatctcattgtggttttgatttgcatttctttaattatcagtgatgttgaatttgttttcatatgcttgttgactgcatgtatgtcttcaattgaaaagtgtctgttcatgtcctttgcccagcttttaatggggttgtttgttttttcttgtaaatttaagtttttatagaagctagatattagacttttgtcagatgcatagtttgcaaatattttctcccattctgtagattgtttactctgttcatagtttcttttgctgtgcaaaacccccttagtttaattagatcccatttgttgcatttgtttttggtgtcttcatcatgaaatctttgctagttcccatgtccagaatggttttgcctagatcatcttccagggtttttataattttgggttttacatttcattctttaatccactttgagttgatttacaccattttatatgctgtaaggaagtgatccagttttaatcttctgcatatagctagccagttatcccagcaccatttattaaacagggagtcctttcctcattgtttgtttttgtcagctttgttgaagatcagatggttgaagatgtgtggccttatttttgagctctctattctgttttattggtctttgtgtctggttttgtacgagtcttatgctgttttggttactgtagccctgtaatatagtttgaagtcaggtagcatgatacctccagctctgtttttttttgtttaggattgccttggctattctgcttctttttggttccatatgaattttaaaatagtttctttctagttctatgaagaatgtcatgggtaatttgataagaatagcattgaatctataaattgctttgggcagtatggccattttaataacattgattccccttgaaaactggcaacaaggatgccctcttttaccacttgcatccaacatagtattggaagtcctggccagaacaatcaggcaagagaaagaaataaaaagcattcaaataggaagagaggaagtcaaattatccctttttgcagatgacatgattctatatctagacaaccccctagtcttagcccaaaaattaagctgataaacaatttcagcaaagtttcaggatacaaaatcaacatacaaaaatcactagcatttcaacacaccaacaacagtcatctaaaattttattcatttgagtcttttctccttaggcagacacagggtttgttgattttgtttaccttttcaaacaaactctttgtttcattgatcttttatattttttaagtctctattttgtttatgtcttctctaaccatcattatttctttgtgtctactaattttgggttcagtttgttcctgtttttcttggtccttgaggtgcaacattgggttatctgagatctttgttcttttttgaaataggcatttcaaaattgctataaacttccttctgagtactgcttttgctgtatcccataggttttggtaggttgtgttttcattttcatttgtctctagaaatttttaaatttcccttttagtttctctattgactccttgattgatgaggattatgttgtttaatttgcatgtgtttgtagtttccaaagtttctcttgttattgatttctagcttcataccactgtgatcagaaacaatacttaatatgatttcaatttttttgaatttgttaagacatattttgtggcctaccatatggtctattctggagagcattctatatgtactagagaagaatgcgtacactggtgctactggataaaaagttctatatttatctattaggtccattttgtctaaagcacagatcaattccagtattttcttattaatttctttctggttgatctttccatctttgaaagaggggtattgaagtctcctactattattgttttggtatctattttttaccttcatgttgattaattaatatttgctttatatgtttaggtgttccaatgttgagtgcacatgtatttacaattgctatgacttcttaatgacttgagctctttaataatgactttctttgtctcttgtgaaaatttttgtgttaagttttttctatctagaataagtattgtccctgtcctctgttggtattatttgcaaggaatatcttctttcatcccctttgagggatgaaagcctatgtgcattcttaaggtgaaagtgtgtctcttgtgtgcagcatatagtgagagcttattttttttttctctttgcaacttttattttaggttcgggggtacatgtgcagttttattacatgagtaaattgcatgtcactggggtttggagcacaaatgaattcgtcactaaagtagtgagcatagtacctgataggtagtttttcagtcttcaccctcctcccatcctccaccctcaagtataccctggtttctattgttcccttctttgtgtccatgtgtactcagtgtttagctcccacttataactgagaacatgtggtatttggttttctgttcctacattaatttgcttaggataatggccttcatctgcatccagattgctgcaaaggacgtagtttcattgtttgtttatttttgaggcagtcttgctctgtcacctaggctggtgtagtggcaccatctcatttcacaaacctctgcctcctgggttcaagcaattctcctgcctcagcttccccagtagatgggattacaggtgcccaccaccaagcctggctaatgtttgtatttttagtagagacagggtttcaccatgttggccagggtggtcttgaacttctgacatcaggtgatctgctcaccttggcctctcaaagtgctgggattacaggtgtgagccactgtgcctggccaatttcattcttttttatggctgcatagtattccatggtgtatatataccacattttctttatccaattcaccactgatgggtatctagattggttccataactttgctattgtgaatagtagagctttgtttttttaatccattcagccactctgtgttttaattgaacaatttaatctgtttacattcagagtaattattgatatgccaaggacttactactgccattttgttaaatgttttctgattgctttatagcttatttgttctttttttcctctcctgtttatctttgtgatttagtgatttcctttagtgctaaatttgtattcttttatctttatcatttgtatatctgctttagtattctgctttgtggttaccacaaggcttacataaaatgtcttatagttataatagactattttatactaataacaacttaaattcagttgcatacaaaaactgtagaattctaccctcccaccagcgtgtgtgtgtgtgtatagtatatatagtatatatacacacatatatatgtatatgtacatatattcatatttcttttttacctttctgagttgggtgcaaccagttaatattttttgatgtcacaattttcatctttttagcttacatatttcttaatactttattgtagctatcattatttttgactgttttgaattttaacttccatcctagagttatatatacctatgttttgcagagtgccatagtagtatcagggtattctggatttgaatatatatctacctttatcagtggatttttttactttttatgttttcatgttagtaatgttctttttttctacttgaaaaactcccttaagcatttcttatagagcagctctagtggtgaagaattccctaaacttttgcttgtctgggaaagagtttatttcttctttttttctgaaggacagctttgctgtgtatagtatttttggctggcaggtttctgtgcatcagcactttgaatatatcatcccattctctcctggcctgcaggatttctactgagaaatctgctgatagtctaatggagattcccttatatgtaacatgatatttttatcttgctgcttttaatatcatttgtctttggtttttgacagtaattataaagggacctctttgggttaactctgtttggagattgttgagcttcatatatttggatgttcatctgtcttccaaggcttgaaacatgttatttttcatttatcaaaaacttatattcactaattttaaagttttacattttttaaaaaagttatctatattgtccttttattttttgaacatattaatcacaatttagaatcatttttgttaattccattacacttcttggttctatttatgttccctgtttttttccttggtttttatggttttcagtaaattcttacctttcaatatatttatttatttttggtttaatgccagacattgcgtattcaaaattttagagatgactcgaggctctggatgttgtgtgattttcctccagataatatattctttgttttgggcagtgagatagacaagggtaaaggttaggagaaattcacctaaattcagttaggatgaagtaatttaaatctaggtttcagtcctttggagaactgacctttttcaaattaccatttattttagatacatgaaaaccattttctatttattttaatacagtaaataaattctctgttaccagcaccttctaggaacatttttcttgttcttagttgggtgagagagctagatcttaacctatagttagggaggaaagattgtctcattggctcttccctgtccaccagaaacagcaatgctaacttatatgccattacttcttggcagtatattcaagaattgggttctttgagaaggactaacttacatgcccattacttcttggcagtatattcaagagttgggttccttgggagaaaagaggaggactagaaggttgctgaaatgttaacatgagacatcttactttttttgttgtctctgactcaattttgcaagataatgtgaagagggtgtgaatgactttctaggtctactcagtcaagtcacttttgctaccacctttgaaatgctgatgtacctatacatataactactaatactgatactgatctgagggagaccccttactgcagggttggtggccttgctgagtggaaaacctcattctctattcctaatagaacagcttgaacttttggaatggagtagaggcccttgaagggaggactatagctataaagcttgtgaagatatttgacaacatgcttaatatgatacatttaactggttggaaaggataagcttttttgtgttcacagcaagcaaagatgattataatccaattttagttttaaaattgggacttgtgctgaaacaccatagtcttgtgggcagagttgattgcttgtagaagacttctattagcaatcacttatactcggtgatcctcagaaaactgtttcttaaattatttttttcccttgattaatattttaatatggatacaaacaccaagaggccttctttgcagtgtcatttatctcttacaaatctcattttcctttcagatattatcagacgcgacacagcctcttgaaagtcagaatttttctcctgtggtgagagatgtaagttatgagcagaatattttttcataaatgtatttttttactaccatttttgctctcttctctctgtttctctctctctccatctttgtcattctgtcttttctctatcagtcatgacaccttgtttgtaggactaaaatgtggatactaaagaaatgtcttgtgtcatatacatttaggcagtgattacatccaacggtacattacctgataaatataaatatatccagacactccgggagagcaggtaagtattgttcaaatcctcttgttttatacatttattcttatcttggaattcatgagtattttttaatgcttcaatgggagttcttaatattaggtttgtacactatataaattttgataatgtttgaaaatgtaggaaatccaaactaagtggctggctgaaaccactgtgactgtttcccacttaacagtagtatagcgtcattttggctttctaatagcctgtgtagacgtatgggtaagcaagcaaaaatccaaaaggattgtccttcaggtagcttcttgtttttctctagcttttgatacatgatcatgcctcggttattttctcaaaggaaaatgagcacactagcctttctgtggtgctttcagtttgggttttagttgaggatttggactctcttttgtttagtataatgttcagggaagtgaattcgtggttacaaattaagagagttgtgaataaacaattgatgaaagtaagaggtagttttctccagggattcttattagggggaaggtgccaaaaagtaatgtagcaaatctactcaagagaatagttactttagaaaggtctttagctaaggcccttgtttccttaattgattttactacttaaagtttgaaaacattgattttatttttttggaatattctggtctcctccagccaattgcccttgatctgtgtttatatctactctgctggtcttttcacattaaaaaaggaacccatgtactgaagtatcaggagaaaccagagttggaaaaaaaaataacacaactgttttatcttctttttcagtggttctactacaagcagtaaacaaagagatctgcccagatcaagcgtactatagttaggcttttccatatgtgctggtttgattatagttactttttaatgtgtcagtgaaatatactggcataagacactaagtatttactttttttctttgtgaataagagaagctagctctataaccctgtaggagagtttcttacaagtaaaacttcacatctgtttgtttttcatagctctatgttttggttctcatcatccgatggtcttttacactactgaaactagtgacatgtctaacatgactggtcaagatgatatcttctctcttatgtgtcatatttcatgaagcagcaggtttacttggctccgattgctagaacttgctgccctctttcctgatggaaacttgtgccaagatactgataacaaaggttgcattttgaaaggaaatttactttatattgaaccctgccttattcttctcttcttttcatcacttagcacgtgattattaacctggagtctggggtttgggggtactgatgggctggacagtattagaatctaggagtgagtttctgttttgagcatttggtatttggaatgttttaattatacaaactatagaaaatattgttagacagaaaatataataaccgaataatagtggttctctctggaaggtgaaatgggagatggtagtagttaaatgagacctcttcacatcagtaatatgtgtgttgggatacagaaggacaggaattttttatacttatatttatttttatttgttaaaaattaatgaaaagttttttgggttaatgttcaagagacattatggcctaaagattctaaataacagagcctacttcagtccctggcactaggccatttctatggtttcatgttgataacagggaatttgattcaaaaaatatttaatgagtaaattccatatttaagacaaaatgtgctgagcattaatgggtttgtaaagatgggtaagtcttagatgctgccttggaggtcatagtttgtgggaaagacagaaagtcatctgcctggaaggtattaatataaattccagcagaatagactgttcaagaattaaaataatgcctaacactagtcttcagcttctacatttaacccatggcatgtgtgactcaaagtcatggttttgctattttcaagtgactctaaagatatatgacaaaacaatttctaatatggataataatattctcttcctcaaattgttgtcttctgggattgttgtgaggaataaataaggtttccaaggtaaaaccccttgtatgattttgaaccacatcttctggaaggataaaatgtgagaccaagttactcaatcacaagtaagccaagaaaatgtccagcatctgcacctcactagtaatactgggaattcatgaaatacatctatacgtctgtagatatatatctcagagatgtcaggtctattgttcttcattgtacaaaatgtaaaataattaaagaaacatgttgttatagggaactcattcatgttgaaattcttattttaagtaatgtgaagagaaaatgttgcttcattatttaggttttagtatgatttgtggaatgcaagattctgttatttaaaaaaaaatttttaattagccccttcccttttttttttttttttttttttttaaatagggaacgtgctcagtcactttcaatgggcaccaggagaaaggttttggttcttggatctggctacatatctgagcctgtattagaatatttatcaagagatggcaatatagaaataacagtaggtaaataagccctaatttttgaagaagaaaaatgtattttatagcgtgttgacttctacattttagaagactactttgtttctgaaatttgaaaatttatttttggagatgttaaaatatattttactctgacatatctaaaaatgtgacttttaattactgaaataggatctgacatgaagaatcaaattgaacagttaggcaagaaatataatattaatcctgttagcatggacatttgtaaacaagaagagaagctgggcttcttggtggcaaaacaggatcttgtcatcaggtcagtatctggcaagaaatgagtttcatatttttgttgttatttcttttctcatttttctttctctttcacttctcctctcttcatactcctttggagtaaagtttttaattgaagtatgggtccagaaggtgcacaaatcataagtatatagctcaatgaattttcacaaagtgaacacattctgtgatgattatttatgtcaggaaacaagagtatcagtaattccccagaagccctcctggtgcctctttctattcacttaccccatcctctaagggtaaacactatcctggcttctgacatttagtttctgcctgctttcagacttcatataattggaatcattcaatatatcctcatttgtggcttccttcttccattcaatgttgtgattacaagagtcatttgtgctattgtctgaaatgataatttattcattctcattgctgtatagtatttcattatgtgaatataccattattatttatttgactactatagatatttaggttgtttctggattttggctattataagtcacactgctatacacatttcggtatttgtcttttagtgaccatttcactaaaacagtaattatacatttctgttgggtatgtacttagaagtgggattgcaggtcataaggtattaatgttttatatacttaagtgtctattttatcctttggagttataaagcttaaatggaataagtagattatttagccttatgaccattttaagtctcatcatagcaacagatcctgaaatagagtattactaacaaaaatgcagatctaacatctagacacagaattacatgagattcactttataactaaatttggttttgatatttgagtacatatgtacacaaagaaaggaacaacagacatcagggcctgcttaagggtaaaaggtgggaggagggtgaggatcgaaaaactacccattgggtactatgcttattacctagataatgaaataatctgtacaccatacccccataacatacaatttacctgtataacaaacctgcacatgtacccctgaacctaaaataaaagtaataaataaataaataaataaatttggttttgaaatgtcaaagtgatgaaaatagtcattcattcctagaattttcccaagatgagaagaagaaagttatctgttccctcatagtgattacctagtcactttcacagatcagattcaaattgtatctttggaggaagttctgtgatgttctgctttaaaaaaaaaaaagcaggcttggaaaacatataatgaaaggtagttctgtaggctggcatttttacatttctcttgatatctttaaatacaggctaactttatgcacttaaaatgttactcaatttatataaatgttatgagcgttaatttttatttagaaaaaattgactaatgaccaactgaacctctctgatattaccatgaaaagaaattctgattcaaaatagaatgatctagccttctgagtagaacaataatttattttgccacacagaggtatctaaatatatcatttttaattaaaagtaaaataacatgataaaatccatttggaacagaatctgaaataaaatgtgacaagataatggacaaagacatcaatctcttaatatgtccaaattttaaaatttagcaaactagtggccttcaataggatttagcattagcaaaaactgttttatatttaatgttttctcatttggtgtattttctatgctgaataatggtaatgccattaaacaatgttatcctataacattccttaaattttttttttcttttttttgagacagtctcattttgtcacccaggctggaatgcagtggcacaatcttggctcactgcaaccttcacctcccagactcaagtgatcctcccacctcaaccacccaagtagctgggactacaggcacttgccactatgcctagctaaagtttgtatttttagtagcaatgggatatcaccatgttgctcaggctggtcttgaactcctcaagcaatctacttgcctcagcctctcaaagtgtcaggattacaggcataagccaccatgcctggctaattgttttattttttaagttggggaaagaatgttttgatataaccacagtgatagggactacaaaaaaatgagtaaagaatatgtttcacaaaaaatgtttagtaatatgtacaattctcatagttataatttttcaaatttagtttttaatttaatacaaagtagtaattgatttcaagtttctttgaaaattccacatgtattttgtgtgtatttttcacaagtgacattttcgaccttttggagtaattgggttggagtcaacttaaataatttaagagtatgtcccattgagtgcttactgtatcatattcgagacgaattatgaactctaatcccttcgtctaaaaaagtaccacaggcaaagtcctatcattctacttctcagcctattataaggccaaagttttaaaatattattagatcttagtaaaattttgattgagcaagtttaatatggagaatgtgatttaaaaatttctgtttaacctttaagatgatgacattgcacattttctattaatatttggttctaatagttttgcgagtattttgctcagtggtttccaaaagtctactaaatttgttagcagtaattatttttggtttaaggaaacaaaaacacagagaccaggagcttttgatactgataaaagacatacacaattttattctgcattataataaatttaaatagcaatactctattctgttgacctgaggaggaataaatgcctgaaaagtggttttattgtttttttttttttatgaagtagagaaaatctgaactatgcagaatgaatttgacaagtacaatgatatagtgtaatattgtttctgttggtttttatttttttttgttggtactgtagacagctttaccaaataggcattaaaaaacaaattaaatattatcatgtaattaacagagtattttattattcctagaatagaatatggatatgctcatttttataacactcatatttatgaaaatttaatttcttttgattatgcagtcccagagccaaattattataccacagccaaatttgttgaaaaccgaatcaaagttgagctttgagtacagcatcatccattgttcaccatgtggatttttagccattttgatagcatttacactgatcaacaccgttaaagcctgtctcctccgtggacccttcctggagtctactcaataatgtaatacattagctgtagaaatatagggatgaatggagcaaattccattaaatgttataaatatataactggatgcatgcagaggaaggatatctggctttggggaaatctgggaaagctttgtggaggaaaataactctttagaaatttcttaaagaggcaggtataatttggattggggttttctgtcgtgggctttccactaaaatagggctgctgttctttttaccttcacatcttccaaatggctatcaggaagtgtcaaactaggtcaagatggcccatgtattacattattgagtagactccaagaagggcccaaggaggagataggctttcatggtgttgatcggtgtaaaggtatcagaatggctaaaaatccacagggtgaacaacagatgatgctgtactcaaagctcaactttgattgggttttcaacaattttggctttgggattataatttggtgctgggtctgcataatcacaagatatcaaattttcataaatatgagtgttatataaatgagtatatccatagtctattgtaggaataataaaataccctgtcaattacatgacaatatttaagttgttttttactgactatttggtaaagctgtctgcagtaccaccaaaaaaataaaaaccaacataaacaatatcatactacatcattgtacttgtcaaattaactctgcatacttcagattttctctagttcattaaaaaaacaaaataaacaataaaaccacttctcaggcctttattcctcaacaggtaaatagaatagagtattgctatttagatgtgataacagggtgaatttattctgtggcccacagggagtcattgatcacgtctttccaagggaatatttatttattttactttcaacttttattttacaatcaggggttacatgtgcacatttattacaaaggtatattgtgtgattctgaggtctggagtatcaatgaatctgtcaccaagtagcaagcatagtacccagtaggttgtttttcagcccctgctccacttcttatctcttccctcttgtagtcctcagtgtctattgtttccatctttaattccatgtgtacccagtgttcagctcccacttacttgtaagtgagagcctgcagtatttggttttctgtttctgtgttaggtcgcataagataatagtttccagttgcatccatgttgctgcaaagaacactatttcattcttttttatggctatgtagtattcaatggtgtatatgtactacattttttaatccaattcacggtttttgtttttttatttttgtttcattttgttttgttttattgagacagggtctcactctattgcccaggctagtgtgcagtgctgtgatctcaactcactgcagccttgacctcctgagttcaagtgatcctcccacttcagtctctggagtagctgggactacaggcatgcgccaacatgctgggccaatttttgtatttgttgtagagatgatgtttcaccatcttgtccaagctggtctcgactcctgggcccaagcgactcacctgtctttggcctcccaaagttctgggattacaggcatgaggcacagcacctggccccaatcccactattgatgggcatgtcaattgattccatgtctttgttattgcgaatagtgctgtgatgaatatgtgagtgcctgtgtctttttggtagaataatctattttcctttggatagataaccagtaatgcgattgctaagtcgaatgatagttcaacctttagctctttgagaaatctctgaactatcctctgtagcagctgaactaatttacattcccaccaacagtgtataagtgttcccttttctgagagaataatttaatcagaacagtaccttaagatgcagctttaatacagttcaagtaccttagaaagtagagttgaaaaagggagtttctgattagtttcttcctgttttcaagcttgttgccttatgtattgcaccctcttgtggccaaggcctgcatcacaaacaaagttaacatggtcactgcaagctacatcacaccagcactaaaagaattggaaaagaggtaagttttaatcattttaaacaaactgagtggatccatggcattgtttttattttatatccaagttttcctttttcctctttccacctgctgcagtgtggaagatgctggcatcacaatcattggtgaattgggattggaccctggtctggatcacatgttagcaatggaaacaatagataaagccaaggaagtgggagccacggtgagcccaatttgtacccaaaggcaacattaaaatgtctaaattcttgtgtacagaatcaaagtatgagttataatatagcaacacctttaatcaaagttaaagattttttttttttgagactgagttttgctcttgttgcccaggctggagtgcaacggcacaatctcggctcactgcaacctccatctcccaggttcaagtaatactcctgcttcagcctcccaagtagctgggattacaggcacgcaccaccatgcccggctaattttgtatttttagtagagatggggtttctccatgttggtcaggccagtctcaaactcctgacctcaggtcatctgcccccctcagcctcccagaaattttaaataatatcttataactgcaaaaatatccatacaggaaaacaatgtccaagtcttaggtagcaaccaatttttggagaaacagatgctactataaaattttggcttaatacaaattttagactaccttgaagattctgtaataagatttgacttttaaaagtaagaagaatctttgggtatcagcaatctgttgatatgaaatatcttttatgaaatagtacctttggcgaggtactattgggtgtaaatgggatgattcagattgttttaggttttttgaatgtgtgtgtatatatatgtattaaaattatatatatgtaaatatataatttgaatacaaaagttagtaggaactgactaagcagaattgaactaagagttggatttttaactgaacaaaatgttagtcactatttttacagtttttttaaaaaaacatatatataaaattaactgttatttaatcaaatggtacatgctttgaagatgtatttttgaatgccttagtaactttaagaatgaaaaactgtagcaagtttactccattcatatcattgtcttcaattaattacttagacattctctaagttgtgaattcttccttatttattgattctttaatttatagattgaatcatatatttcctactgtggtgggcttccagcccctgaacattcaaacaatccattgagatataaatttagctggagtccagtgggagttttgatgaatgtaatgcagtctgccacctatctgctcgatggaaaggtgaggaatcttatccaagcatagtgatatcaacctgcaataacagcctgagacagtctaaatactccctgatcccaaaccctttaataatggtgatgctgcctgtctccattatctcctcctgtgttgtttcccagtgtatattttctgttctatttagcacggtatccttgctgttcctagcaaagcctgtgcctattctttgccctaggccttcttgcaagttgtttctgctattcaaaaattcacttctctatatatgtaaacactggttatctcttgatttatttcagcatgacaaacattttctgagtacctactatgtgctggactttgggcaagttacccaagactgtcacagagaatgaaattcatgacctcagaaagctcacagctggactggggatggggctcttggaatggagggtttggtgtgctggggaaagaaactataatttcttccccagctgccatgtaacagagcataagaactctccaagtttaatattattttcattattttacaataagaaagctatggctcagagagcatgtccaaggtcatttggctgagattcaaacccaggtctaactaactcttaaggctttttttctccttttgccagattgcctaatggctggaaagacagacatgggaataagtaattataatttaatagggaaatttccaaagtatgttaaagtggctcagagctacagatgggagaacagtttaattggccttggtgaaggggttgggggaaggaggatggggaaaagaaaaactagtggagaagaggatgtgcttggccttgaaggatatgagggtgccagctaggcagggatgttagcactcatggtatgacagttttataaggggaaaaatcaggtttagggagttaagataacagctacacttaaagagatggcaagtgacagagccagtatccaaatcatgtctgtctgacttcatagtttatgctcatagtttggagaaaaatgaaacgagactcagagtgcctctttgaattgtgtgttgtaggcacctcattcaccttgccctagtcctggccctgcagggagtgatgagtaatatgatcacatagatgtgtcttaaaaagattattttagcatcaatattttacattgtcaaggtatttgtgaatcgaaatgtatttcaggagtagagagaaagaatttcttaagcaacaaggtgaatgctgttattagccatggtcttttgtgcctgtgatatagagcataactcatacttggcttggaattacaaggggagtttaaaaagtaccagcgcctcatccccaaagattctgatttgcctgggtttgaggattttttaaatcttcctagtgattctaatatgcagctgaatttgagaacaactgatttaatgatcagagaatcacagctgcacttaaaatagtactaaacaagcaggacactttagcttgatgccggaatgatgtgtgacatgtttcaggataataaacaggatcaatgagttctggtctaattcctggcatccctcataacatagccaaagaccaggcagttgaaaagatctaccggatgtcagtttcctgatgtgtaatgtgagcactgatgttccatccaattctttaattctatgaatcatgaaggtttggaccagaatcttagtctgatgttttaccaagcacacatccttttacatgctacagcctatctcctattgcattccaacagctccttttaccttctgcaatcttaagagactggcctatgtttggtataaattaacatgtgaaggcaggacaaattcttgcctcagttgagtaaaacacttatttttaggaggccttataaatgctaaagatggtaacaacgaaagggtcagcagagtgcaaccatgtaaaagaaagagcaaggaacttggtatctgtgggcctggctttgtgctctagctctgccattactggttgtgagctttgggttccttgtctgaagataaagacaatcatgccagccttccagtgttcttagtcagtgaggtcacatagattgcaatctgcctaacagcagattgtcagccatgtaataaatctagcaaatattagctacataaggaaattagtggttgtgagagtttaagacaaacaagaggttattttcaggtagcagaagttgtatttgttgaattcgaagttctcctctttcatcctaaatgtcttttgtaaaaatgagtgctttttgcatctctctgacactggaggataccaagagaaaatatgacacttttctttgctaaaatgatacttttttcaaggtcccacctgcgtttcctcttgtggatgaagctttttgtgaccactccagcctctgttcactattattttgcctgaactgaatcaaagaatcagacactttgtttgtctgtgtcttaagaattttctaggtgctgcattattcagtcagtaattcaagtgtatcagtctgtttctcaagccagattgttgaacaacttaaaacctctcttgtattcttgcccttcactccctctgccatcaccaccctccactgtggagcgggtcttaagtaattcacagaatcataatgggaaaccaagtcacctctctgacttctgcttgaatttgaagacttaaacttttccagaaattggtcagtggcctatgaatttgaatgtgtctcattgtcttacatctttgtagtaatttctagccacttgtactatttctaacttctaatttgattcctaacaattctgattttcttccacatattcggagacagtgatttcatcttctcttagtttcattcccctcacagtggtggtgtacttttttacattgttggcatttgggagtgatatggcagagctttgccatcatcttctgcagtgtttcccagtctaacctggcaaaacatgtagtctaagtgagaagtgtgcttgctctgcaaacatcgctttaagataagttttctagccctctatccattgcctgccaccaccttgactgcagcacccaaccctttggaaatcaagagctaccactatcttcctttgcctgtaccaattctttatgcaactttcagtgcatcccaggtggcataatccagaatgctgtactttgtcaatatccctttcaaaatactgatccagaaataagcatgattaaggggacagtgtagttgaaggaacacagacattgcagtcaaatagaactgggttcagatttgcctttgggtgactcagcctcagttttcacctctgaaaaatgagttgtttcatggattaaagataatgcccagtacaaggcactgggcacatagtaggccctgaaaaaatgattgtatatttccttattagtttagtgatcaagcttactgtgattatttgaatatatatgatatatttattcaaatataacagtgattctctctacttgggagatttgtatttgtgtttgtgtgtatgtgtgaaagagttggacctagagaagcttgcaacagcagtgaataatgagcctgagcattgtcagagcagaaagaaatgtatattctctgcaaaacagacaaatatgtttctgttgtcattcaaattattaataaaataataaacaagaaagaacttattgttgaactacattatataccactagaaatacaactacgaatgaatatcaaatgacaatgattacccagtaatttttgggtcaggttgcttagccacctgccattctcactaaatgaatcattgccattcagctgacatttctccatcttctttgtaaggatttcatgatagacttatgaacttgctgctttatttatgattggtaagctagaatattgtggtcattattgaatttttctttcagaaattttcctgactttcaaacaagcaaacaaaacttaagaattgtcatgggttatagaaatgagatgggtgtgaagaccctaaaagtcaacgtttgcttttttgtttttaagagatggtgtcttgctgtgtcgctcaggctattctcaaactcctgggctcaggccatcctcctgcctcagcctcccgagtgaagtagctgggattacaggtgggcaccatcatgcccaacatttgctttcttagattttttcaaaaatttagaattctattaaataaactggttttaaagaagtagttcagaaatcatcccccctttggaaatgtttactctgcagtctagtagggttcacaagaaaattaaatggaaggcattcctcacatccttcattcagattttcacttttgaaattttaaatggtgcttggcactctccaaagtgagtctctgaactgaaaagactccagtggtcctaacattgcagtcagcatagcccttttctttctggggcttcatccacagataaatgagtttccattttctctctcatacaaaggcacgttcagaacccatctactaataaggcatgagaatcattaagtttagccaaaggcctcttcaggcaaatcctgtgaatccaccttaaagtacattggcagctccctggtacagtgttattttgtgcagctttagagaaactggtcagaaattgtttccagtggtacaataaggataaaatttagttccaccagcagcaacaaccagaattctcagacctaattttatcactccagaatgctttcctttatctcaacaataataataagccaataaactagaagaaaatttagtcttttaattaaaattagagacaaagaaatccttcattgcattttgagtatgtgtgatgggggtaggggtatgggaaagagaagtactgagatatcaaaagatttttatttcatcactcatgttattcccttcacaatttgtgcgatcttcaatgcatagatagtaactgctcaataaatttagctgtatgaatgaaatgtatgtgactgatgcctaatgtggcatttagaaggctcatgaaagtaaaagaaaagaatgagaatattgattgttgtttgagcatagaagccatttaaataattatctgttgcttttagcttatttaagtttattttttaaacatttttatataaaataattttgtttattttttaaacattttttatttttaaatatttagaaaataaattttttaaaaattaaaatgtttattttaaattttttttctaaatgtttatattttttaaaaaggcaaactctagtgggaaataatttagtgccattttatccaggggaaggaaaaaaattgaaaataagaccttatgtttagaaaaactaaatgttttaaagtcaaattttaaagcttgagagaagaattgctttgtgaattatgatattagtagaacagcttaagtataggatgatgattatggtggtttcattatttttattttttttttcaaatttctgttgctttctttgttcgtataatgcagataagccctaaaagaaaggaacagctgtaaccccatgtgtgccttctcaatgttcctcaggttgtgaatgttgcaggaggcatctcctttcttgatgccgttacgtccatggatttttttccaggattaaatttggaaggctatcctaacagagacagtacgaaatatgctgagatttatggcatttcttctgctcacactttgttgcgggggacactgagatataaggtaagcagctggactttagattcaacatgttgccagttgtgatgtaagtgatgtgtacacttaatttttcttagtagtcaaaataattaagatgataaaccaagttggagtctattttttcaggaatcttatgattgattgaatattttaggagactcacctcaaaataaaaagataattaattgactcattaggagttggggacaggcagggcttcaggcttagtgagagttggagatcaagtgacttccccaggacctgacttctcgtaacgtctcagctcttctttccagtattcacaggagtttcaagtgctgtgcgatggcagagtggtggcagtgctcccagcctctcaggatcaaattcagtgggaataaaaaatgataccgttctaatagccaaaaatacgatttttgtctcattagctccctaaggtcagtgggataaaatgtgcccatctactttggcctatgttatatgtcctagaactggagatggagtcaatgtcattaaaaaaacatggactggggcgggaggaaacggacgggatgaaaggggcttccccagagcaaatccaaaatgtttttaaaccacaaaatggggagtacattctaggagcattaatgcaaatgtccactacacatttattgatcacaaaaaaagcagttacaagtgtgacctaatcaagacaactggtgtaatagttttctagcataatatcacaaattgggtagcttgaaacaataggggtttattctctcaccattcaggaggccagaagtctgaaatcaagatgtgtgtgagtagggttggtgccttttggaggcttgactttgagacacatccatccaatttgtgcatcagtttcatatcaccttcttctgtgtgtctcactgtgttgcatcctcttcttattagggcactagtcattggacttagggttcaccctaaatccaggttgatttcatattgaggtccttaattaattctatctgcaaacattcaagtgaggccatattctgaggctgtggatgcccatgaatttggcgacagggccctaaactcaacctaatgtaaccagcatacctaaaatacatgtatttctcaggatcagagacctgactgcaaatctgtctgtcttgtgagatttcctggctaaggccagagaggggatttgtctcatgtgtgttatctagtattctctaattatctttttatctggaagatgtaaggcacatggacctgtcagtgatgacccttcaatcttgtgcctgcttttctgtcgttctcttatctttttttgctggctgttccttttgcctggaacttttcctccaggtatttgtatcatcctccttcgggctttattcttttcttctctgcagccctgcccctggtaaccatggttttattctctatctctgcatcttttactttcgtaaaaaaaaattctgcatgtaagtgagatcatgcaatattttactttttgtgtctcacttattccacttagcataatgtcttccaggttcacctgcctatctgttcttaaaaagaaaacaaccactatatttcttactcttgagagcttctgttacatttatgggcacttagcatttacttctcagcattttaagttataattttatgtatatttttcttatctcccaaaagagattgtaagctcctagagagcaggtgcttgtactccatgaatatgattttttcattgcccaggaatcaaatgagacatcagtgataatgtcctaccgagatctaggagctatggctcttctaccagcctttaggcacagtagaaattctagtctgaacttatgcatactatccccagctctggcagatttcttgatctattttcttcaatttgttcaaagaatatgagtaaaaacttttttttttggccagcagttaggatatatagctttcagtatagtcttttgacaaacatatagatccaggaatgaaggattttaatagatttaaacattgaataaaggtctcaacaaggtgtgcgagtgggagccatccccatggattccttgaggatggaagatatcacagtgttcgtaatcatgagggccaccatcctgggcaatgccagcaatccattggtaaccctaaaattctttccagtattggagatgagataccatcattcaaatatgacagagaggccttgaccactctccttctctgcctgcctgtcacctgcttgtccttctagacgcaggttaatagcaccttcctgtggtatcctccctaactccttccccttgaaccacccagccccatctatgtgagttccccttcttttgtgctcccacagctatgttttcctctaacctagcacctattaatataatactgtactacaatttagtcatcagtggtgatgcatttttgcttaccatggactaggcattatttgcttgacttatctgacccttctgttgactgcagtttctgcatggatgagactgcaattttttcatctctgtggcctcagtccctagcacaagtctgacatatggcagcagtttgctaaatgcttattgatgaatgaatacataaaataaattcatttctaggggtcagcataaaaatatatttctgagattaataaatagctcagaaatcagtattttagatgttggataaagggatctctagcacctatcaggtcccagaagcgagctaaacaagctagtgatattacttttctgtttctcctctttgaagtatatttgcatttgaattagagcaaatgagaattctttttttagtaacctttgaacagtttgaacaatataacctttagaaatgggaaaattgctagttggccagacattcagtagtgaaatatctgacgttacaggaactattttatattttaaaacctctgttgctccctagaattatttccctctctctcttttcttctctctctctctgcttgagcagtaattgcagataagataaatttcttatcttagccttctcaccctgcttcccatttgcacactggggaataaaccattatggtctttcttcacttcatctattattcagaatctatttatccagccatttgttctcttttagggattggaatgctgaataatggattggaatgttgaataatgttgaataccttcctatggtatcctccctaactccttccccttgaaccacccagccccatctatgtgagttccccttcttttgtgctcccacagctatgttttcctctaacctagcacctattaatataatactctactacagtttagtcgtcagtggtgacgcatttttgttcaccatggactaggcattatttgtttgacttatctgccccgaagtgggcattgggatgaagaatatgcacacaaataaaatataatataacataatataaagatacaatttccctgctttcaataccttcagtacagcagctgaatagacaattaattatgagctgcattaattgccatgataaaggtatatacaaaatgctacaagaagggagtgcccacttgtgagtgctaggggactcacaagaggtgctaaactgggtctgaagggaggagaaaacatttcataaatgaagaagtgtggccacaacattccagttacaggagcagcatgtacagaggccctggtttctggaagaacacagcacatttggggaatagtgaaagtggagatctgtgagttaggctagatgagcatgttgtgatgggttttatataatataccaaggacctcaggtttgctttgtagacaatgaaggacatatgaggaatttttatcagagtgatgatatcatcatatcttagaaaaatctctgattcctacctctttttctgctctccatatatcttgggggtggtggctctaattagtagattggattatctgcttttgaggaggatattcttacaagttcctccttccattataagaaagagctcaactgactgttctgttaaataatactctgctgagctgacagcatataacacataagaaaatttgcccttatgctgcccgaaaattccattcacatgttacccagaaacagaagtaaagacctaagttttgcaaggtcttttatgtaaaccagtagaattgttatgacactattgatctcagtttgagcttgaaattggttgtatctatatcctcaatacttttttggtgggttgtggggaggggtttctagtaaagacaggaaaacctgctaggcaaattctagaagagctgtagtactcatatcatcaatataaacagttttctaaattagttacaaagatatatccctcttttcaaaaatattttaagttttttcattggaagagaaaaattttatatactgcttattaattcttgtttcagggatatatgaaagctttgaatggatttgtaaaattaggtcttataaacagagaagcgcttcctgcctttagacctgaggccaaccctctcacctgggtgagtgactccgaattacgttctatctgctccttttcagaaataccttctgaaatggtcgggggtgaactctgctttggatctgcaaaagatcctattttttagcttatgagcaggaagataaatcctaactccaaagccccctttcccttcactgttgtggaagaatggagagggtgtggggctgacacatatcaggcctgtttcccgcagcaacttataccatttgactgctccttgagctgtgataagagatgcctttgctctctgcattaagctctgagctattccagttgtctaggtggtgacccagtgatgggagtcacttggacttcctgaaagtgagaatccaggagagttactagtgtattacagaactggatttccaacctccaaatcttgtcagtactctcatttggttacttataaccatgagtgatttgattttcagttaccgagcttatgcgtctatctcttgttgtaccaaaaccttaaggcctcagagttggaatgaactttggaggcttggaactgaatgcgaagtagagcttctactgactgctcagaattaactcattaccagctggttgttcaacttaaactagtgtgcctgtattccagtagaaatgccagaaatgtatcacaaggagaaaaggaccaatgttgctaattccattacatctcaaaaaaaagttttggtattcttgctagctagtttctttaaaaaattatttttgaattctttatgggtacatagtaggtgtatatatttatggaggacatgatatattttgaaataggcatgcaatgtataataaccccatctgggtaaatggggtatctaccacctcaagcattcatcatttctttgtgttagccagttgcttttacattcattttttttgtgtgcatttgtgaacctttccaatcttaagttttacctttcataaacttacttgttgggtaggagtgaacaagtatctgcagagagaacctggggttgaggcagctggtttgggaaggttggtgcagacattcttagggatggtcttttttgtaaaggaccagattataaaggttttagactttatgggccacacggtctctgtcagtaccactcagttctcccatagacgatacataaaccaatgagcatgactgtgtttcaataaaacttttatttaataaaacagacaatgagccaaatttggtctgtgaaccagtttgttgactcctgatctagagcatagcttttcctttgggaacctagaaaaagtcttgttttgaaagtagaaaaaactatggctactgtttaagtattcttcctttgagattttattacctgcaaggactttctcatctcttgaacctacaaaagaacctgggttgctactggcaaccactgcagtaagccttttccactttatcctggtcaaaatggactgctggagggtagggggagaatggataaaaacatagtgatagagaggatatacaaagacagggaatgtgctggtaagaactgcaccatctgttttgtgttgagttgatcttaaaacccatttttattggtgaaatatcgtttggaatagcagctatttattgatgtgtaggtatgcagatgtgtgtatgtgtgtgtatgtgttacaatttcctatttccaattatttttaattttaaaatttgaggtgtatttgaagttcagtgttttagtaggataatttttcagtcaattaaaaaaaaatttgttagggacttagaaatattgtatttctttttctaaccagaaacaactcctctgtgacctagttgggatttcaccctcctctgagcatgatgtgttgaaggaagctgttcttaagaaactaggaggagacaatacccagttggaggctgctgaatggtaggcacccaccactcaacttagagcaaaatatactggatcaatgattgctaatttctactcagaaaaagttaaatattttacatttgtctttgattaattcgttgctctaatgtgggtagagagattaccatgtgccatgttcatgtgggcataaagagtagataaagagaggagctcaatggcaattagaatttgagaaatgcttatctcgaaacactttaccactcagttcccaagcatagtggggtatttttgctttcctgtaggagatttggagtagtcatagaatattacatgaaaacattctttcatatgaccgctgtggcatcctgctggtatacctaatgttggagaactaacctataaattaatctcataaagatctttgtaagaacatgggattacaatccatcatctctgccctgctccagggagcagtgcctgggtgagtctggagaagattctgaaggttaaaacattgctattgttattgttattttacattttaggagaatcctacaacttgagttaccaccttgtaattctgccctttcaagattaaattcaaggagctcccttttgcttcccaaatgagaacatgtataggacttgaactaatgcagccatctcctgtgcttttcaggttgggcttacttggggatgaacaagttcctcaggcagagtccattctggatgccctctccaagcatttggtcatgaagctttcctatggtaggccatgaagtattagctaaatatacctaaagaagaatcataagatagagtcagtgcagataatcgtattggagggttggccccaagcagcaggaatgggaagatgtgatatgaagaaaagggcaattggactatttttccagagggaggaatttgagaattattaggtaaaaataaacttagttattatgggaaaggaaaaaatagttttactatttatttatttagttagttagttagttattttttgagacggtgtctcacgctatcaccaggctggagtgcagtggctccatctcggctcactgtacctctgcctcccaggttcaagagattctcctgcctcagcctcccgagtagctgggactacagttgcctgccaccatgcccagctaatttttgtatttttagtagagacaaggtttcaccatgttggccaggatggtcttcatctcctgacctcgtgatccgcccaccttagcctcccaaagtgctgggattacaggcatgagccaccgcgcccgacattactttttttttttaatgcattaggcaaatgccaaagaagcatttttggagcacactaaaaggcctctatagcagcgcttttgtgtctgtaaacaggatatggtgacatgaatccggataaaggaatgagatcagagttctaatttccttgaggttttcactttgactttgggctaaaaagctgtgtaaataattgtctagtgatcatatataaaaggagggaaaacttttaagaccagaagggcaggaggagatgacagacaatatttgagttgtaatgatagtggtatttttacttatctttatttgttttaaggtcctgaagaaaaagatatgattgtgatgagagacagctttggaatcagacatccttctggacatttagaacataaaacgattgatcttgtggcttatggggacatcaatggcttttcagccatggctaaaaccgtggggttacccaccgccatggcagccaaaatgttgcttgatggtaagtctgttcatttgaggtgtaagcctgtttctgtttcacctccaagccatttggtgcctggacatcaagtaattgctcttgagcgtaacacaggcatttactaaaagtgaaccatcttagaagatccaagcgcggactgcctgggaggtccccttttgatgagtaaagcatgtgggctttggagatagacctgacttccgcttccgctcagttccaaggtggctgagtgatgggctgcaagttactcagcacctttgagcaagagtttcctcatatttaaagtgggtattgccatccctgtttcagagttgttgtgacactaactgatataatacatgaagctgctacctcatagaggggtccccagggaatggtaacttccttcctttctttctttggtttttgccatgttgggagtgataactctaaaatagaaaaagatattcactccagattctcaagaatataggttaagttttattagtcttcatatctcagcatattattagtactcaataaatattttagactaaatgaataaaaatttcattcctccactctccctagaagatcacatagacattcaataagcattgcctctgtttgaccctcaagaaagaatgaggggagtagaaaatatcaaagcatataaatgtgaaataatgaaaaaaaaatgtatagtagcatatcagctaatgtcaaattaaatgaccaacttagttattgaggaatcaatgaagaagtaagtgataaaagttggggtcattgtggcctctagtgagagtgcccaggttctgtgtttcagtcatactaacaggtgcctgtgcccatataatcaaacctgttacaactcattctgttggtgtcttgccttttttaaaaaaaaatgacattagcaagttaagctgaccagtcagatttactttacttcttactttactttgttctctgattgtcctggcagaaattaatgcagttaagtgtacaagttctgacaatatccctaggtccggctgtgctttcaatctaaaatcaactccttttccttacaaagtgttgaataagtaataatttactaagggacgtttttaagcagctacttaaaaaatgcctaggcctccaagaattcattgactctgagtttgcttctttgtggagactcttgatggggaaattaacctctgacacaacctttgaaatggtaattacacatttattttcttccaggtgaaattggagccaaaggcctaatggggcccttttcaaaggagatctatggaccaatattggagcgaattaaagcagaaggcattatatatactacacagagtacaattaaaccataattgggaattatattttgtttttttcttcccaggcaatacacctctgaacatgtgtgtgataaatgggtttgctaatgtgctgttttaaagtataaagcataatatgttttggttaacacaatgtactttttgaactataaatctttattttaatatggaaatgtttggaacaggagatgcaagccactaacagagaactttaataattctaccctgtattttataaatacgtatgtgaaagtgatgatcatgctatttgttaatttattgtgccactttttttcttttgttttttttttgttgttgttgttttttgagatggagtctcgctctgtcacccaggctggagtgcagtggcgcgatctcggctcactgcaagctccgcctcctgggttcacgccattctcctgcctcagcctcccaagtagctgggactacaggcacccgccaccacgcccggctaaattttttgtatttttcagtagagacggggtttcaccatgttagccaggatgtccacttttttcttaaatgaacatattgtcttataaccagcaaatgaagttttacttttttagtcagaaaaaatttcgtcataaagctatatttttatatttttaaaattgatacagttagataccttcagattacctaagttaagtttttgccaaaatgctattttatgccctttttgtaaagttatgatttattttgtaaatatagttccttatagttttctcttgagtgtttggacatgagaatagataagattaaattacaatttacaagatcacttcattaggcacaggaaaagtatttaacaaaattcaacaccctttcacaataaaaaataaaaaataaaaaaccctcaacaaactaggaatagaaggaaacttcctcaacctgataaagggcatctatgaagaacccatggttagcatcatacttaatggtgaaggactggattatttccccctgagatcagaaacaagacaaggatgtctgctttggccatttctattcaacttgtactggacagtcaagaaagggcaattaggcaagaaaataaaataaaagtcatctaggttggaaaggaagaagtataactaaatagagatgggagacgatatggtggttttgtgcatagtaatccaagggaaaactatagaaaatccaaaggcatgatttattaataaataaattcagcaaagttacaggatacaggattaaatatgcagaaattggttatctttcaatatagtatcaatgaataaatcaaaaatgatgttaagaaaacaatttaattttcaacaatggtcaagcttggtggcttgtgcctgtaatcccaacactttgggagtctgaggcagaaagactgctagaggccaggagaccagcctgggaaacagtgagatcccatctctacaaaaacaaacaaacaaaaaacaacaagcaaacaaaaaacagctggatgtgatgacactcatctgtcgtctcagctactcagaggctgaggtgtaaggatggcttgagccccaaagttcgaagctgcaatgagctataactgggtcactgcaatccagcctggatgacagagttagaccttgactcaaaaaaaacaaaaaagaaaagaaaaaaattgacagcatcagaaagagtaaaataccaatataattgagggtccagaaatattgatcacatttatcatcaactgacttttgaaaatagtgctaagacaattcaatgggggcaaaatagtctttttcattaaatgatgcttggaaacatggatatccacatgcaaaataatgaaattggactcctacctcatattgcatataaaaattaactcaaagttgacctaagatctaaatgtaagagctaatattgtaagtatcttcaaagaaaacatggcgtaaatctttctgactttgaattaggcaatgatttcttaaataaatgacactaaaaacacaagcaacaaaagaagaaaataggtaaattaaaattaaaaccatttgtgtttccaaggacaatatcaagaaagcaaaaatacaacccacagaatgagataattttttttaaaatcatgtattagttaagggaggaatttgtatttagaatatgtaaagaactgttacaataataattaaaaggcatataattcaatttttaaaaatcaacaagagatctgaattaacagttctccaaagaagatacacaaaaggccaataagcacatgaaaaggttctcaacatcactagccatcagggaaatgcaaatgagatctacaatgagatatcacttaacactcactggaatagatatataatcaaagtgataatatcaagtgttggtgaggatgcagagaaattggaaccctcatgcactgttggtggatacccacgtgatataaaaacaaatgtctacacaaaaacttgtgcacagatgttctttgcagcattatttataatagcccaaaattgaaacagccctaatttctatcaactaacaagtgaataaacaaaacgtggcatatccatacaaggaatattattcagcaataaaaaaagagatgaagtactgatatgcaccacaacatggaaaaaccttgaaaacatatcaagtgaagaatgccagacatgaaaggccacatattatatgatttcatttatatgaaatgtccagaacaggcgaatatatatagagagaaggcagattagtggttgcttaggctgaagggttggggaggacaaaggggtgactgctaattagtatggggtttcttttttggggtgatgtaaacgttgtaaagttgattgtggtaatggtggcacaactctgaatatactaaaaaccactgaattgtatactttgaatgtttgaactgtgtggtatgtagattttatctcaagttgttataaaaaattaaattacaaatgcaatttgtttgtttgcttcttggggaaaaatctcttgcctttagagatacaaagtaagcacctttttaaccttcacaaattgtgctcatatcctggcacgtgaaactaattctgcccttgaaagaagcaggtactacatgtgaggtatatgacagttgagaaaacatctcctgctatttgaaaacaatcaaaattgattaaggcctggtcccacctgtcacagataatatatggagatcaataatctattaataagtaaaaacattttgttgtggaatttttcttaccatgacattcagttcgacccaattgattaggaaatacaactcgtgtcatattgatttcactatctaaaacggcgcccagattaattgggaactgagaaactagcactttgtgtcagaactgggatccagttgagctctcagtgtagttaactagtttgtgctttatgatgacaatgagaattaaaggggatttgatttgaagagctgatttaatgttagtgttaaacgtggtaatagaaaaggcaatgtcagtaagcttgaatacagctatatgtaattaaacactacattatccctcaataacaaattttcctgcagtacctttaataataaaggtggtggtgcacatgttctcactcataggtaggaaatgaacaatgagaacacttggacccaggaagaggaacatcacacaccggggcctgtcgtggggtaggggaaggggagagggatagcattaggagatatacctaatgtaaatgacgagttaatgggtgcagcgcaccaacatggcacatgtatacatatgtaacaatcctacacgttgtgcacatgtaccctagaacttaaagtataaaataataataaatgtggtggtgataggatttgggaatgaatgagcaggttatggttataggcatatcaacatatagcttcaccaggagtccacaattatttttctgtgaaagtcagatataaacattttaggct

SEQ ID NO: 10 - DNA, 5825 bp, Homo sapiens
agtttcttctaggaattcggagcctcccctgcagcgactcggaagattcgaggcggcgggggacaagtcggcgccccagagcggacgagtcaccaggtgtcaagatgctgcaagtacataggactggactgggcaggctgggggtcagcctctccaagggtcttcaccacaaagctgtgttggccgtccggagggaggatgtgaacgcctgggagagaagggccccgctagctcccaagcacatcaaaggcatcaccaatctgggatacaaggtcttgatacagccttcgaatcggcgggccattcatgataaggactatgtcaaagctggtggcattcttcaggaggatatttctgaagcttgtctaattttaggagttaaaagacctccagaggaaaaattaatgtccaggaagacttatgcatttttctcccacacaataaaagctcaggaggccaatatgggcttgttggatgagattctaaaacaggaaattcgccttattgattatgagaaaatggtggatcatagaggagtacgggtagtggcatttggacagtgggctggtgtggcaggaatgatcaacattttacatggaatgggtttaaggctccttgctttgggacatcacacaccttttatgcacattggcatggctcataactacaggaatagcagtcaggctgtgcaagctgtccgtgatgctggctatgaaatatctttgggtttgatgcctaagtcaataggacccttaacatttgtgttcacaggaactggtaatgtttctaagggagcccaagcaatctttaatgagctaccttgtgaatatgtggagccccatgaattaaaagaagtttcccaaactggagacctcagaaaagtgtatgggacggtgttaagtcgtcatcatcatcttgtcaggaaaacagatgctgtgtatgatcctgcagagtatgacaaacatccggagcgctacataagtcgttttaatactgatattgcaccctatacaacttgcttaattaatggaatctactgggaacaaaacactcctcgcctcctaacccgccaagatgctcagagtctcctggctccgggcaagttctcacctgctggtgtggaaggctgccctgcattaccacacaaactcgtggcaatatgtgacatttcagctgacacaggagggtctatagagtttatgactgagtgtacaacaatagagcatcccttttgcatgtatgatgcagaccagcatattattcatgacagtgttgaaggctcggggatcctgatgtgttccattgacaatttgccggcacagctcccaattgaagctacagaatgctttggagacatgctttacccttatgttgaagaaatgatattatcagacgcgacacagcctcttgaaagtcagaatttttctcctgtggtgagagatgcagtgattacatccaacggtacattacctgataaatataaatatatccagacactccgggagagcagggaacgtgctcagtcactttcaatgggcaccaggagaaaggttttggttcttggatctggctacatatctgagcctgtattagaatatttatcaagagatggcaatatagaaataacagtaggatctgacatgaagaatcaaattgaacagttaggcaagaaatataatattaatcctgttagcatggacatttgtaaacaagaagagaagctgggcttcttggtggcaaaacaggatcttgtcatcagcttgttgccttatgtattgcaccctcttgtggccaaggcctgcatcacaaacaaagttaacatggtcactgcaagctacatcacaccagcactaaaagaattggaaaagagtgtggaagatgctggcatcacaatcattggtgaattgggattggaccctggtctggatcacatgttagcaatggaaacaatagataaagccaaggaagtgggagccacgattgaatcatatatttcctactgtggtgggcttccagcccctgaacattcaaacaatccattgagatataaatttagctggagtccagtgggagttttgatgaatgtaatgcagtctgccacctatctgctcgatggaaaggttgtgaatgttgcaggaggcatctcctttcttgatgccgttacgtccatggatttttttccaggattaaatttggaaggctatcctaacagagacagtacgaaatatgctgagatttatggcatttcttctgctcacactttgttgcgggggacactgagatataagggatatatgaaagctttgaatggatttgtaaaattaggtcttataaacagagaagcgcttcctgcctttagacctgaggccaaccctctcacctggaaacaactcctctgtgacctagttgggatttcaccctcctctgagcatgatgtgttgaaggaagctgttcttaagaaactaggaggagacaatacccagttggaggctgctgaatggttgggcttacttggggatgaacaagttcctcaggcagagtccattctggatgccctctccaagcatttggtcatgaagctttcctatggtcctgaagaaaaagatatgattgtgatgagagacagctttggaatcagacatccttctggacatttagaacataaaacgattgatcttgtggcttatggggacatcaatggcttttcagccatggctaaaaccgtggggttacccaccgccatggcagccaaaatgttgcttgatggtgaaattggagccaaaggcctaatggggcccttttcaaaggagatctatggaccaatattggagcgaattaaagcagaaggcattatatatactacacagagtacaattaaaccataattgggaattatattttgtttttttcttcccaggcaatacacctctgaacatgtgtgtgataaatgggtttgctaatgtgctgttttaaagtataaagcataatatgttttggttaacacaatgtactttttgaactataaatctttattttaatatggaaatgtttggaacaggagatgcaagccactaacagagaactttaataattctaccctgtattttataaatacgtatgtgaaagtgatgatcatgctatttgttaatttattgtgccactttttttcttttgttttttttttgttgttgttgttttttgagatggagtctcgctctgtcacccaggctggagtgcagtggcgcgatctcggctcactgcaagctccgcctcctgggttcacgccattctcctgcctcagcctcccaagtagctgggactacaggcacccgccaccacgcccggctaaattttttgtatttttcagtagagacggggtttcaccatgttagccaggatgtccacttttttcttaaatgaacatattgtcttataaccagcaaatgaagttttacttttttagtcagaaaaaatttcgtcataaagctatatttttatatttttaaaattgatacagttagataccttcagattacctaagttaagtttttgccaaaatgctattttatgccctttttgtaaagttatgatttattttgtaaatatagttccttatagttttctcttgagtgtttggacatgagaatagataagattaaattacaatttacaagatcacttcattaggcacaggaaaagtatttaacaaaattcaacaccctttcacaataaaaaataaaaaataaaaaaccctcaacaaactaggaatagaaggaaacttcctcaacctgataaagggcatctatgaagaacccatggttagcatcatacttaatggtgaaggactggattatttccccctgagatcagaaacaagacaaggatgtctgctttggccatttctattcaacttgtactggacagtcaagaaagggcaattaggcaagaaaataaaataaaagtcatctaggttggaaaggaagaagtataactaaatagagatgggagacgatatggtggttttgtgcatagtaatccaagggaaaactatagaaaatccaaaggcatgatttattaataaataaattcagcaaagttacaggatacaggattaaatatgcagaaattggttatctttcaatatagtatcaatgaataaatcaaaaatgatgttaagaaaacaatttaattttcaacaatggtcaagcttggtggcttgtgcctgtaatcccaacactttgggagtctgaggcagaaagactgctagaggccaggagaccagcctgggaaacagtgagatcccatctctacaaaaacaaacaaacaaaaaacaacaagcaaacaaaaaacagctggatgtgatgacactcatctgtcgtctcagctactcagaggctgaggtgtaaggatggcttgagccccaaagttcgaagctgcaatgagctataactgggtcactgcaatccagcctggatgacagagttagaccttgactcaaaaaaaacaaaaaagaaaagaaaaaaattgacagcatcagaaagagtaaaataccaatataattgagggtccagaaatattgatcacatttatcatcaactgacttttgaaaatagtgctaagacaattcaatgggggcaaaatagtctttttcattaaatgatgcttggaaacatggatatccacatgcaaaataatgaaattggactcctacctcatattgcatataaaaattaactcaaagttgacctaagatctaaatgtaagagctaatattgtaagtatcttcaaagaaaacatggcgtaaatctttctgactttgaattaggcaatgatttcttaaataaatgacactaaaaacacaagcaacaaaagaagaaaataggtaaattaaaattaaaaccatttgtgtttccaaggacaatatcaagaaagcaaaaatacaacccacagaatgagataattttttttaaaatcatgtattagttaagggaggaatttgtatttagaatatgtaaagaactgttacaataataattaaaaggcatataattcaatttttaaaaatcaacaagagatctgaattaacagttctccaaagaagatacacaaaaggccaataagcacatgaaaaggttctcaacatcactagccatcagggaaatgcaaatgagatctacaatgagatatcacttaacactcactggaatagatatataatcaaagtgataatatcaagtgttggtgaggatgcagagaaattggaaccctcatgcactgttggtggatacccacgtgatataaaaacaaatgtctacacaaaaacttgtgcacagatgttctttgcagcattatttataatagcccaaaattgaaacagccctaatttctatcaactaacaagtgaataaacaaaacgtggcatatccatacaaggaatattattcagcaataaaaaaagagatgaagtactgatatgcaccacaacatggaaaaaccttgaaaacatatcaagtgaagaatgccagacatgaaaggccacatattatatgatttcatttatatgaaatgtccagaacaggcgaatatatatagagagaaggcagattagtggttgcttaggctgaagggttggggaggacaaaggggtgactgctaattagtatggggtttcttttttggggtgatgtaaacgttgtaaagttgattgtggtaatggtggcacaactctgaatatactaaaaaccactgaattgtatactttgaatgtttgaactgtgtggtatgtagattttatctcaagttgttataaaaaattaaattacaaa

SEQ ID NO: 11 - protein, 926 aa, Homo sapiens
MLQVHRTGLGRLGVSLSKGLHHKAVLAVRREDVNAWERRAPLAPKHIKGITNLGYKVLIQPSNRRAIHDKDYVKAGGILQEDISEACLILGVKRPPEEKLMSRKTYAFFSHTIKAQEANMGLLDEILKQEIRLIDYEKMVDHRGVRVVAFGQWAGVAGMINILHGMGLRLLALGHHTPFMHIGMAHNYRNSSQAVQAVRDAGYEISLGLMPKSIGPLTFVFTGTGNVSKGAQAIFNELPCEYVEPHELKEVSQTGDLRKVYGTVLSRHHHLVRKTDAVYDPAEYDKHPERYISRFNTDIAPYTTCLINGIYWEQNTPRLLTRQDAQSLLAPGKFSPAGVEGCPALPHKLVAICDISADTGGSIEFMTECTTIEHPFCMYDADQHIIHDSVEGSGILMCSIDNLPAQLPIEATECFGDMLYPYVEEMILSDATQPLESQNFSPVVRDAVITSNGTLPDKYKYIQTLRESRERAQSLSMGTRRKVLVLGSGYISEPVLEYLSRDGNIEITVGSDMKNQIEQLGKKYNINPVSMDICKQEEKLGFLVAKQDLVISLLPYVLHPLVAKACITNKVNMVTASYITPALKELEKSVEDAGITIIGELGLDPGLDHMLAMETIDKAKEVGATIESYISYCGGLPAPEHSNNPLRYKFSWSPVGVLMNVMQSATYLLDGKVVNVAGGISFLDAVTSMDFFPGLNLEGYPNRDSTKYAEIYGISSAHTLLRGTLRYKGYMKALNGFVKLGLINREALPAFRPEANPLTWKQLLCDLVGISPSSEHDVLKEAVLKKLGGDNTQLEAAEWLGLLGDEQVPQAESILDALSKHLVMKLSYGPEEKDMIVMRDSFGIRHPSGHLEHKTIDLVAYGDINGFSAMAKTVGLPTAMAAKMLLDGEIGAKGLMGPFSKEIYGPILERIKAEGIIYTTQSTIKP

SEQ ID NO: 12 - DNA, 20 bp, Artificial Sequence
  AASS forward primer used in the Examples.
aacagtggtcgcctcctaac

SEQ ID NO: 13 - DNA, 20 bp, Artificial Sequence
  AASS reverse primer used in the Examples.
tttgtgtggtaatgcagggc

SEQ ID NO: 14 - DNA, 20 bp, Artificial Sequence
  GAPDH forward primer used in the Examples.
gaaggtgaaggtcggagtca

SEQ ID NO: 15 - DNA, 20 bp, Artificial Sequence
  GAPDH reverse primer used in the Examples.
ttgaggtcaatgaaggggtc

SEQ ID NO: 16 - DNA, 16 bp, Artificial Sequence
  nucleic acid sequence of AON1, a nucleic acid silencing molecule sequence according to SEQ ID NO 1
ucagaaagatttacgc

SEQ ID NO: 17 - DNA, 16 bp, Artificial Sequence
  nucleic acid sequence of AON2, a nucleic acid silencing molecule sequence according to SEQ ID NO 2
uaggtaatctgaaggu

SEQ ID NO: 18 - DNA, 16 bp, Artificial Sequence
  nucleic acid sequence of AON3, a nucleic acid silencing molecule sequence according to SEQ ID NO 3
gaaagatttacgccau

SEQ ID NO: 19 - DNA, 16 bp, Artificial Sequence
  nucleic acid sequence of AON4, a nucleic acid silencing molecule sequence according to SEQ ID NO 4
cgggttaggaggcgag

SEQ ID NO: 20 - DNA, 16 bp, Artificial Sequence
  nucleic acid sequence of AON5, a nucleic acid silencing molecule sequence according to SEQ ID NO 5
ccugtgcctaatgaag

SEQ ID NO: 21 - DNA, 16 bp, Artificial Sequence
  nucleic acid sequence of AON6, a nucleic acid silencing molecule sequence according to SEQ ID NO 6
uguagcgctccggaug

SEQ ID NO: 22 - DNA, 20 bp, Artificial Sequence
  nucleic acid sequence of AON7, a nucleic acid silencing molecule sequence according to SEQ ID NO 7. AON7 is an extended (20mer) version of AON4
ggcgggttaggaggcgagga

SEQ ID NO: 23 - DNA, 20 bp, Artificial Sequence
  nucleic acid sequence of AON8, a nucleic acid silencing molecule sequence according to SEQ ID NO 8. AON8 is an extended (20mer) version of AON6
uauguagcgctccggauguu

SEQ ID NO: 24 - DNA, 16 bp, Artificial Sequence
  nucleic acid sequence of a LNA-modified AON1
  misc_feature (1)..(3): LNA
  misc_feature (4)..(13): phosphorothioated DNA backbone
  misc_feature (14)..(16): LNA
ucagaaagatttacgc

SEQ ID NO: 25 - DNA, 16 bp, Artificial Sequence
  nucleic acid sequence of a LNA-modified AON2
  misc_feature (1)..(3): LNA
  misc_feature (4)..(13): phosphorothioated DNA backbone
  misc_feature (14)..(16): LNA
uaggtaatctgaaggu

SEQ ID NO: 26 - DNA, 16 bp, Artificial Sequence
  nucleic acid sequence of a LNA-modified AON3
  misc_feature (1)..(3): LNA
  misc_feature (4)..(13): phosphorothioated DNA backbone
  misc_feature (14)..(16): LNA
gaaagatttacgccau

SEQ ID NO: 27 - DNA, 17 bp, Artificial Sequence
  nucleic acid sequence of a LNA-modified AON4
  misc_feature (1)..(3): LNA
  misc_feature (4)..(13): phosphorothioated DNA backbone
  misc_feature (14)..(16): LNA
cggggttaggaggcgag

SEQ ID NO: 28 - DNA, 16 bp, Artificial Sequence
  nucleic acid sequence of a MOE -modified AON4
  misc_feature (1)..(3): MOE
  misc_feature (4)..(13): phosphorothioated DNA backbone
  misc_feature (14)..(16): MOE
cgggttaggaggcgag

SEQ ID NO: 29 - DNA, 16 bp, Artificial Sequence
  nucleic acid sequence of a LNA-modified AON5
  misc_feature (1)..(3): LNA
  misc_feature (4)..(13): phosphorothioated DNA backbone
  misc_feature (14)..(16): LNA
ccugtgcctaatgaag

SEQ ID NO: 30 - DNA, 16 bp, Artificial Sequence
  nucleic acid sequence of a LNA-modified AON6
  misc_feature (1)..(3): LNA
  misc_feature (4)..(13): phosphorothioated DNA backbone
  misc_feature (14)..(16): LNA
uguagcgctccggaug

SEQ ID NO: 31 - DNA, 16 bp, Artificial Sequence
  nucleic acid sequence of a MOE -modified AON6
  misc_feature (1)..(3): MOE
  misc_feature (4)..(13): phosphorothioated DNA backbone
  misc_feature (14)..(16): MOE
uguagcgctccggaug

SEQ ID NO: 32 - DNA, 20 bp, Artificial Sequence
  nucleic acid sequence of a MOE-modified AON7
  misc_feature (1)..(5): MOE
  misc_feature (4)..(15): phosphorothioated DNA backbone
  misc_feature (16)..(20): MOE
ggcgggttaggaggcgagga

SEQ ID NO: 33 - DNA, 20 bp, Artificial Sequence
  nucleic acid sequence of a MOE -modified AON8
  misc_feature (1)..(5): MOE
  misc_feature (6)..(15): phosphorothioated DNA backbone
  misc_feature (16)..(20): MOE
uauguagcgctccggauguu

SEQ ID NO: 34 - DNA, 19 bp, Artificial Sequence
  nucleic acid silencing molecule targeting AASS
  misc_feature (10)..(10): n is t or u
  misc_feature (12)..(12): n is t or u
  misc_feature (18)..(18): n is t or u
ccaggagacncngagcanc

SEQ ID NO: 35 - DNA, 20 bp, Artificial Sequence
  nucleic acid silencing molecule targeting AASS
  misc_feature (5)..(6): n is t or u
  misc_feature (14)..(14): n is t or u
  misc_feature (19)..(19): n is t or u
cgacnnaacaccgncccana

SEQ ID NO: 36 - DNA, 20 bp, Artificial Sequence
  nucleic acid silencing molecule targeting AASS
  misc_feature (1)..(2): n is t or u
  misc_feature (5)..(5): n is t or u
  misc_feature (13)..(13): n is t or u
  misc_feature (16)..(16): n is t or u
  misc_feature (19)..(19): n is t or u
nnccngacaagangangang

SEQ ID NO: 37 - DNA, 19 bp, Artificial Sequence
  nucleic acid sequence of MOE7, a nucleic acid silencing molecule sequence according to SEQ ID NO 34
ccaggagactctgagcauc

SEQ ID NO: 38 - DNA, 20 bp, Artificial Sequence
  nucleic acid sequence of MOE8, a nucleic acid silencing molecule sequence according to SEQ ID NO 35
cgacutaacaccgtcccaua

SEQ ID NO: 39 - DNA, 20 bp, Artificial Sequence
  nucleic acid sequence of MOE9, a nucleic acid silencing molecule sequence according to SEQ ID NO 36
uuccugacaagatgaugaug

SEQ ID NO: 40 - DNA, 19 bp, Artificial Sequence
  nucleic acid sequence of a MOE -modified MOE7
  misc_feature (1)..(5): MOE
  misc_feature (6)..(14): phosphorothioated DNA backbone
  misc_feature (15)..(19): MOE
ccaggagactctgagcauc

SEQ ID NO: 41 - DNA, 20 bp, Artificial Sequence
  nucleic acid sequence of a MOE -modified MOE8
  misc_feature (1)..(5): MOE
  misc_feature (6)..(15): phosphorothioated DNA backbone
  misc_feature (16)..(20): MOE
cgacutaacaccgtcccaua

SEQ ID NO: 42 - DNA, 20 bp, Artificial Sequence
  nucleic acid sequence of a MOE -modified MOE9
  misc_feature (1)..(5): MOE
  misc_feature (6)..(14): phosphorothioated
  misc_feature (16)..(20): MOE
uuccugacaagatgaugaug

SEQ ID NO: 43 - DNA, 20 bp, Artificial Sequence
  Aass mouse forward primer used in the Examples
agggtccagattcaagagct

SEQ ID NO: 44 - DNA, 20 bp, Artificial Sequence
  Aass mouse reverse primer used in the Examples
ctctaagcctggccagtctc

SEQ ID NO: 45 - DNA, 20 bp, Artificial Sequence
  Hprt1 mouse forward primer used in the Examples
ccctggttaagcagtacagc

SEQ ID NO: 46 - DNA, 21 bp, Artificial Sequence
  Hprt1 mouse reverse primer used in the Examples
acaaagtctggcctgtatcca

(19) United States
(12) Patent Application Publication
(10) Pub. No.: US 2024/0043843 A1
(43) Pub. Date: Feb. 8, 2024

(54) TREATMENT FOR LYSINE DEGRADATION-ASSOCIATED DISORDERS

(71) Applicant: UCL BUSINESS LTD, London (GB)

(72) Inventors: Philippa Mills, London (GB); Haiyan Zhou, London (GB); Paul Gissen, London (GB); Reddy Sreekanth Vootukuri, London (GB)

(21) Appl. No.: 18/269,141

(22) PCT Filed: Dec. 22, 2021
(86) PCT No.: PCT/GB2021/053413

§ 371 Date: Jun. 22, 2023

(30) Foreign Application Priority Data
Dec. 23, 2020 (GB) 2020549.8

(51) Int. Cl. C12N 15/113; A61K 31/4415; A61K 31/205; A61K 31/198; A61K 31/712; A61K 31/7125; A61P 3/00

(52) U.S. Cl. CPC C12N 15/1137; A61K 31/4415; A61K 31/205; A61K 31/198; A61K 31/712; A61K 31/7125; A61P 3/00; C12Y 105/01009

(57) ABSTRACT

The disclosure relates to a method of treating a disorder of the saccharopine pathway of lysine degradation in a subject, and a composition for use in said method. The disclosure also relates to a nucleic acid silencing molecule that reduces the expression of an enzyme involved in the saccharopine pathway of lysine degradation.

### FIELD OF THE DISCLOSURE

The disclosure relates to a method of treating a disorder of the saccharopine pathway of lysine degradation in a subject, and a composition for use in said method. The disclosure also relates to a nucleic acid silencing molecule that reduces the expression of an enzyme involved in the saccharopine pathway of lysine degradation.

### BACKGROUND

Several inherited metabolic disorders affect the saccharopine pathway of lysine degradation. These disorders arise when an individual has a mutation in one of the genes which encode the enzymes involved in this pathway. The mutation results in a block in the pathway and causes an accumulation of metabolites upstream of the step catalysed by the affected enzyme. In many cases, the metabolites are thought to be neurotoxic.

In more detail, mutations in ALDH7A1 (Aldehyde Dehydrogenase 7 Family Member A1) may result in a form of pyridoxine-dependent epilepsy. This pyridoxine-dependent epilepsy has an incidence of 1:64,000, and is characterised by intractable neonatal seizures caused by a lack of vitamin B6. Current treatments involve administration of pyridoxine (a form of vitamin B6), dietary restriction of lysine, and/or arginine supplementation. While pyridoxine administration may control the seizures, it may also cause a peripheral neuropathy. In addition, dietary restriction may be burdensome for the patient and their families, and lead to a deficiency in the essential amino acid lysine. Furthermore, 75% of patients have developmental delay that is believed to be caused by accumulation of the ALDH7A1 substrate AASA and other metabolites. Current treatments do not remedy such developmental delay.

Mutations in DHTKD1 (Dehydrogenase E1 and transketolase domain containing 1) may result in 2-aminoadipic 2-ketoadipic aciduria (also known as 2-aminoadipic and 2-ketoadipic aciduria, 2-aminoadipic 2-oxoadipic aciduria; or 2-aminoadipate and 2-oxoadipic aciduria). 2-aminoadipic 2-ketoadipic aciduria is an autosomal recessive disorder that has an incidence of 1:64,000. Symptoms may include seizures, immunodeficiency, developmental delay, mild-to-severe intellectual disability, ataxia, epilepsy, and behavioural disorders, which are not well managed by currently available treatments. Mutations in DHTKD1 may also result in Charcot-Marie-Tooth disease type 2Q. Charcot-Marie-Tooth disease type 2Q is a rare disease that results from an autosomal dominant nonsense mutation. Patients are typically 13 to 25 years old at the onset of symptoms, which include peripheral neuropathy. Currently, only supportive treatments are available. Mutations in DHTKD1 have also been shown to have an association with eosinophilic esophagitis.

Mutations in glutaryl-CoA dehydrogenase may result in glutaric aciduria Type I. Glutaric aciduria Type I has an incidence of 1:100,000 in children worldwide with up to 1:300 newborns affected in the Amish community and the Ojibwa population of Canada. If left untreated, glutaric aciduria Type I results in irreversible striatal damage and a movement disorder, and many affected individuals do not survive to adulthood. Current treatments include a lysine- and tryptophan-restricted diet, and supplementation of arginine, carnitine and riboflavin. However, even if treatment is implemented early, affected individuals may develop kidney disease and/or experience white matter changes.

There is therefore a need for improved treatments for disorders of the saccharopine pathway of lysine degradation.

### SUMMARY OF THE DISCLOSURE

The present inventors have demonstrated that it is possible to reduce gene expression of alpha-aminoadipic semialdehyde synthase (AASS) using a nucleic acid silencing molecule. As AASS is the first enzyme in the saccharopine pathway of lysine degradation, reducing AASS expression may reduce the accumulation of toxic metabolites arising from metabolic disorders affecting the pathway. In this way, symptoms of the disorders may be alleviated.

Use of a nucleic acid silencing molecule that reduces expression of AASS to treat disorders of the saccharopine pathway of lysine degradation is associated with several advantages. Firstly, AASS is the first enzyme in the pathway (see FIG. 1), and so all downstream disorders may be treated by reducing expression of AASS. For example, a nucleic acid silencing molecule that reduces expression AASS may be used to treat a disorder arising from one or more mutations in ALDH7A1, such as pyridoxine-dependent epilepsy. A nucleic acid silencing molecule that reduces expression of AASS may be used to treat a disorder arising from one or more mutations in DHTKD1, such as 2-aminoadipic 2-ketoadipic aciduria, or Charcot-Marie-Tooth disease type 2Q. A nucleic acid silencing molecule that reduces expression of AASS may be used to treat a disorder arising from one or more mutations in glutaryl-CoA dehydrogenase, such as glutaric aciduria Type I.

Secondly, disorders of the saccharopine pathway of lysine degradation are typically treated with a lysine-restricted diet. Such a diet can pose a burden on patients and their families, and can conflict with social and cultural traditions. It can also lead to a deficiency in the essential amino acid lysine. Thus, lysine-restricted diets typically require frequent monitoring by a specialist dietitian. By using a nucleic acid silencing molecule that reduces expression of AASS to reduce the accumulation of toxic metabolites, disorders of the saccharopine pathway of lysine degradation can be treated without dietary restriction. This minimises the burden placed on patients and their families, and ensures that lysine is readily available for protein synthesis.

Thirdly, side effects associated with reduced expression of AASS are likely to be mild. Whilst there is a known disorder which occurs due to mutations in AASS, resulting in hyperlysinaemia (characterised by elevated plasma lysine levels>600 μmol/L), the general consensus is that this is a benign disorder. Approximately 50% of reported probands are asymptomatic and have been detected incidentally and whilst there are some individuals reported with a neurological phenotype there is a lack of a consistent picture leading to the suggestion that hyperlysinaemia is an anecdotal biochemical finding unrelated to the clinical phenotype. This is further supported by recent data from a knockin mouse model for AASS which is reported to have no apparent clinical consequences.

In summary, use of a nucleic acid silencing molecule that reduces expression of AASS may prevent the accumulation of toxic metabolites resulting from disorders of the saccharopine pathway of lysine degradation. Clinical manifestations of disease are therefore alleviated. Side effects are generally minimal, and patients may consume a normal diet.

The disclosure therefore provides a method of treating a disorder of the saccharopine pathway of lysine degradation in a subject, comprising administering to the subject a composition comprising a nucleic acid silencing molecule that reduces the expression of alpha-aminoadipic semialdehyde synthase (AASS). The disclosure also provides:
a composition for use in a method of treating a disorder of the saccharopine pathway of lysine degradation in a subject, wherein the composition comprises a nucleic acid silencing molecule that reduces the expression of alpha-aminoadipic semialdehyde synthase (AASS), and the method comprises administering the nucleic acid silencing molecule to the subject;
a nucleic acid silencing molecule that reduces the expression of alpha-aminoadipic semialdehyde synthase (AASS); and
an in vitro method for reducing expression of AASS in a cell, comprising contacting the cell with the nucleic acid silencing molecule of the disclosure.

### BRIEF DESCRIPTION OF THE FIGURES

FIG. 1: Saccharopine pathway for lysine degradation. AASS: α-aminoadipic semialdehyde synthase; AASA: α-aminoadipic-semialdehyde; AAA: α-Aminoadipic acid; KAA: α-Ketoadipate.

FIG. 2: a, b) Mechanism underlying pyridoxine-dependent epilepsy. Mutations in ALDH7A1 result in the accumulation of toxic metabolites, one of which (P6C) interacts with pyridoxal 5′-phosphate (PLP), the only form of vitamin B6 which can act as a cofactor for more than 70 enzymes. c) Timeline of therapies for pyridoxine-dependent epilepsy. Genetic basis for ALDH7A1-deficiency identified in 2006, vitamin B6 was administered to control seizures. Triple therapy=vitamin B6 and arginine supplementation alongside lysine restriction.

FIG. 3: a) Mechanism underlying α-aminoadipic and α-ketoadipic aciduria and Charcot-Marie-Tooth disease type 2Q. Mutations in DHTKD1 result in the accumulation of toxic metabolites. b) Mechanisms underlying Glutaric aciduria Type 1. Mutations in glutaryl-CoA dehyrogenase result in the accumulation of toxic metabolites such as glutaric acid (GA), 3-OHGA and glutarylcarnitine.

FIG. 4: Effect of antisense oligonucleotide treatment on mRNA expression of AASS in ALDH7A1-deficient fibroblasts. A) LNA modified antisense oligonucleotides suppressed AASS (p<0.05), with AON4 and AON6 having the most dramatic effect (p=0.0001). (b) MOE-modified AON4 and AON6 significantly reduce AASS (p=0.0001)

FIG. 5: Functional assay for ALDH7A1-deficiency by measurement of AASA in fibroblasts using mass spectrometry.

FIG. 6: The levels of AASA in fibroblasts from a) patient 1 (P1) and b) patient 3 (P3) are significantly reduced by the treatment with AON4 in MOE chemistry (p=0.0082 and p=0.0008, respectively). Similarly AON6 in MOE chemistry also reduced AASA levels in P3 (p=0.0007)

FIG. 7: Effect of AONs MOE4, 6, 7, 8 and 9 on AASS mRNA expression in ALDH7A1-deficient patient fibroblasts. Analysis performed using quantitative PCR (q RT-PCR). AONs used at a concentration of 100 nM.

FIG. 8: Effect of AONs MOE4, 6, 7, 8 and 9 on AASS mRNA expression in GCDH-deficient patient fibroblasts. Analysis performed using quantitative PCR (q-RT-PCR). AONs used at a concentration of 100 nM. Average % AASS expression inhibition given above each bar. Statistical significance was calculated using GraphPad Prism

FIG. 9: Initial screening of AONs MOE 6, MOE 6 mouse and MOE 8 on Aass mRNA expression in 3T3 J2 mouse fibroblasts. AONs used at a concentration of 100 nM. Quantitative PCR (qRT-PCR) analysis was used to assess expression levels of Aass mRNA.

FIG. 10: Measurement of glutarylcarnitine (C5DC) using a mass spectrometry based assay. C5DC measured at a concentration of 50 μM using a hydrophilic interaction liquid chromatography (HILIC) method. MRM transition used: 276.2>84.9.

FIG. 11: Level of expression of AASS in HepG2 cells after transfection of AONs MOE 6-9 into cells using a standard transfection method. AONs used at a concentration of 100 nM. Quantitative PCR (q-RT-PCR) analysis used to assess expression levels of AASS mRNA

FIG. 12: Effect of AONs MOE 4, 6 and 8 on AASS expression in HepG2 cells when AONs introduced to the cell using a reverse transfection method. AONs used at a concentration of 100 nM. Quantitative PCR (q-RT-PCR) analysis used to assess levels of AASS mRNA expression. n=3.

FIG. 13: Effect of various concentrations (10 nM, 50 nM, 100 nM) of AONs MOE 4, 6 and 8 on AASS expression in HepG2 cells when AONs introduced to the cell using a reverse transfection method. Quantitative PCR (q-RT-PCR) analysis used to assess levels of AASS mRNA expression.

FIG. 14: Evaluation of half-maximal inhibitory concentration (IC50) of MOE 4, 6 and 8 in HepG2 cells. The IC50 was identified at 1.42 nM, 4.308 nM and 8.86 nM for MOE 4, 6 and 8, respectively. The graphs were generated using GraphPad Prism software for IC50 calculations based on the dose response data generated in FIG. 13.

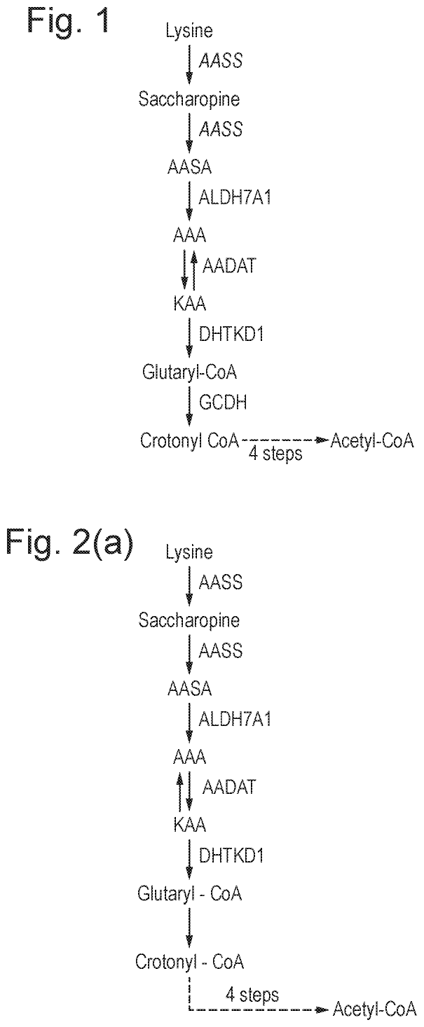

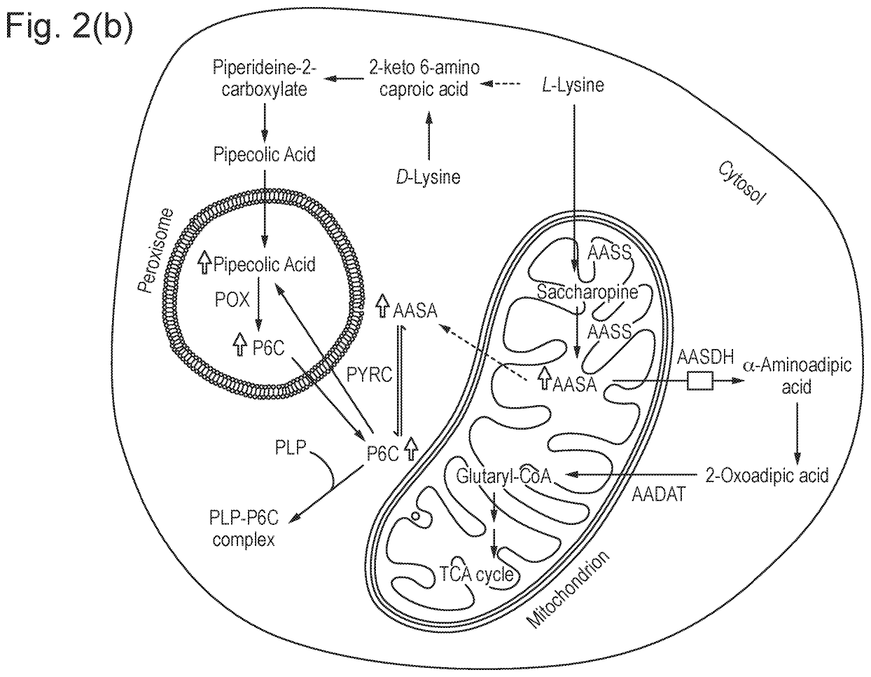

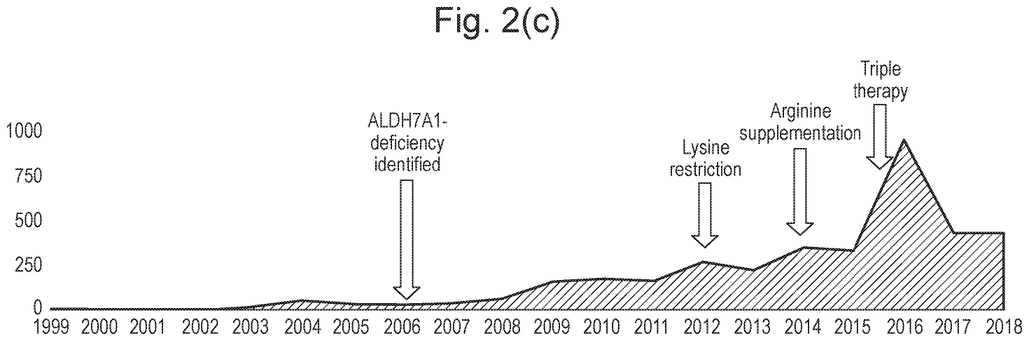

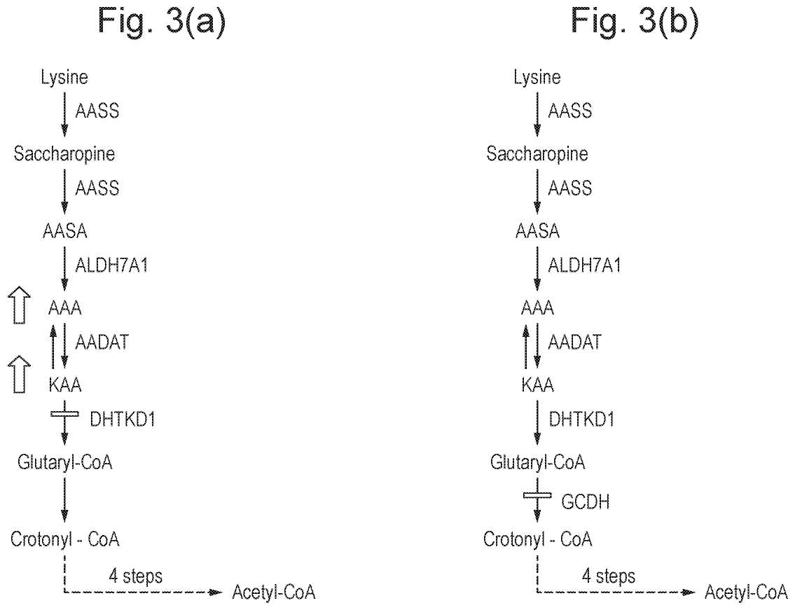

### BRIEF DESCRIPTION OF THE SEQUENCE LISTING

SEQ ID NO: 1—nucleic acid silencing molecule of the invention. Each N may be independently selected from T and U.
SEQ ID NO: 2—nucleic acid silencing molecule of the invention. Each N may be independently selected from T and U.
SEQ ID NO: 3—nucleic acid silencing molecule of the invention. Each N may be independently selected from T and U.
SEQ ID NO: 4—nucleic acid silencing molecule of the invention. Each N may be independently selected from T and U.
SEQ ID NO: 5—nucleic acid silencing molecule of the invention. Each N may be independently selected from T and U.
SEQ ID NO: 6—nucleic acid silencing molecule of the invention. Each N may be independently selected from T and U.
SEQ ID NO: 7—nucleic acid silencing molecule of the invention. SEQ ID NO: 7 is an extended (20mer) version of SEQ ID NO: 4. Each N may be independently selected from T and U.
SEQ ID NO: 8—nucleic acid silencing molecule of the invention. SEQ ID NO: 8 is an extended (20mer) version of SEQ ID NO: 6. Each N may be independently selected from T and U.
SEQ ID NO: 9—nucleic acid sequence of the AASS gene.
SEQ ID NO: 10—nucleic acid sequence of mRNA encoded by the AASS gene.
SEQ ID NO: 11—amino acid sequence of AASS protein.
SEQ ID NO: 12—AASS forward primer used in the Examples.
SEQ ID NO: 13—AASS reverse primer used in the Examples.
SEQ ID NO: 14—GAPDH forward primer used in the Examples.
SEQ ID NO: 15—GAPDH reverse primer used in the Examples.
SEQ ID NO: 16—nucleic acid sequence of AON1, a nucleic acid silencing molecule sequence according to SEQ ID NO: 1.
SEQ ID NO: 17—nucleic acid sequence of AON2, a nucleic acid silencing molecule sequence according to SEQ ID NO: 2.
SEQ ID NO: 18—nucleic acid sequence of AON3, a nucleic acid silencing molecule sequence according to SEQ ID NO: 3.
SEQ ID NO: 19—nucleic acid sequence of AON4, a nucleic acid silencing molecule sequence according to SEQ ID NO: 4.
SEQ ID NO: 20—nucleic acid sequence of AON5, a nucleic acid silencing molecule sequence according to SEQ ID NO: 5.
SEQ ID NO: 21—nucleic acid sequence of AON6, a nucleic acid silencing molecule sequence according to SEQ ID NO: 6.
SEQ ID NO: 22—nucleic acid sequence of AON7, a nucleic acid silencing molecule sequence according to SEQ ID NO: 7. AON7 is an extended (20mer) version of AON4.
SEQ ID NO: 23—nucleic acid sequence of AON8, a nucleic acid silencing molecule sequence according to SEQ ID NO: 8. AON8 is an extended (20mer) version of AON6.
SEQ ID NO: 24—nucleic acid sequence of a LNA-modified AON1.
SEQ ID NO: 25—nucleic acid sequence of a LNA-modified AON2.
SEQ ID NO: 26—nucleic acid sequence of a LNA-modified AON3.
SEQ ID NO: 27—nucleic acid sequence of a LNA-modified AON4.
SEQ ID NO: 28—nucleic acid sequence of a MOE-modified AON4.
SEQ ID NO: 29—nucleic acid sequence of a LNA-modified AON5.
SEQ ID NO: 30—nucleic acid sequence of a LNA-modified AON6.
SEQ ID NO: 31—nucleic acid sequence of a MOE-modified AON6.
SEQ ID NO: 32—nucleic acid sequence of a MOE-modified AON7.
SEQ ID NO: 33—nucleic acid sequence of a MOE-modified AON8.
SEQ ID NO: 34—nucleic acid silencing molecule of the invention. Each N may be independently selected from T and U.
SEQ ID NO: 35—nucleic acid silencing molecule of the invention. Each N may be independently selected from T and U.
SEQ ID NO: 36—nucleic acid silencing molecule of the invention. Each N may be independently selected from T and U.
SEQ ID NO: 37—nucleic acid sequence of MOE7, a nucleic acid silencing molecule sequence according to SEQ ID NO: 34.
SEQ ID NO: 38—nucleic acid sequence of MOE8, a nucleic acid silencing molecule sequence according to SEQ ID NO: 35.
SEQ ID NO: 39—nucleic acid sequence of MOE9, a nucleic acid silencing molecule sequence according to SEQ ID NO: 36.
SEQ ID NO: 40—nucleic acid sequence of a MOE-modified MOE7.
SEQ ID NO: 41—nucleic acid sequence of a MOE-modified MOE8.
SEQ ID NO: 42—nucleic acid sequence of a MOE-modified MOE9.
SEQ ID NO: 43—Aass mouse forward primer used in the Examples.
SEQ ID NO: 44—Aass mouse reverse primer used in the Examples.
SEQ ID NO: 45—Hprt1 mouse forward primer used in the Examples.
SEQ ID NO: 46—Hprt1 mouse reverse primer used in the Examples.

### DETAILED DESCRIPTION

It is to be understood that different applications of the disclosed methods and products may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a cell” includes “cells”, reference to “an antisense oligonucleotide” includes two or more such antisense oligonucleotides, and the like.

In general, the term “comprising” is intended to mean including but not limited to. For example, the phrase “a nucleic acid silencing molecule comprising RNA” should be interpreted to mean that the nucleic acid silencing molecule contains RNA, but that the nucleic acid silencing molecule may contain additional nucleic acids.

In some aspects of the disclosure, the word “comprising” is replaced with the phrase “consisting of”. The term “consisting of” is intended to be limiting. For example, the phrase “a nucleic acid silencing molecule consisting of RNA” should be understood to mean that the nucleic acid silencing molecule contains RNA and no additional nucleic acids.

The terms “protein” and “polypeptide” are used interchangeably herein, and are intended to refer to a polymeric chain of amino acids of any length.

For the purpose of this disclosure, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide residues at nucleotide positions are then compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm.

### Nucleic Acid Silencing Molecule

Disclosed herein is a nucleic acid silencing molecule that reduces the expression of alpha-aminoadipic semialdehyde synthase (AASS).

Such a silencing molecule may be used therapeutically to reduce the expression of AASS. By reducing the expression of AASS, the saccharopine pathway of lysine degradation may be blocked or inhibited at its early steps, because AASS is the first enzyme in the pathway. By blocking or inhibiting the top of the pathway in this way, the formation of downstream metabolites may be prevented or reduced. Accumulation of toxic metabolites, such as neurotoxic metabolites, may thus be prevented or reduced. By preventing or reducing the accumulation of toxic metabolites, disorders of the saccharopine pathway of lysine degradation may be treated. For example, clinical signs or symptoms of such disorders may be reduced or abolished. Clinical signs or symptoms may be stopped from progressing. Treatment of disorders of the saccharopine pathway of lysine degradation using the nucleic acid silencing molecule disclosed herein may have advantages over other treatment methods, such as dietary lysine restriction, as set out above.

Other uses of the nucleic acid silencing molecule disclosed herein may also be envisaged. For example, the nucleic acid silencing molecule may be used to experimentally reduce the expression of AASS in vivo or in vitro. In other words, the nucleic acid silencing molecule may be used as a research tool, for instance to investigate the saccharopine pathway of lysine degradation.

### Reducing the Expression of AASS

The nucleic acid silencing molecule disclosed herein reduces the expression of alpha-aminoadipic semialdehyde synthase (AASS). The nucleic acid silencing molecule may, for example, reduce the expression of AASS mRNA. The nucleic acid silencing molecule may, for example, reduce the expression of AASS protein. The nucleic acid silencing molecule may, for example, reduce the expression of AASS mRNA and AASS protein.

The nucleic acid silencing molecule may, for example, reduce the expression of AASS in a cell. The cell may, for example, be human or from a human cell line. The cell may, for example, be in vivo, ex vivo or in vitro. The cell may be a cell contacted with the nucleic acid silencing molecule. Reduced expression of AASS in a cell may, for example, refer to a reduction in the amount of AASS mRNA and/or AASS protein comprised in the cell. Accordingly, a cell that has reduced expression of AASS may, for example, comprise a reduced amount of AASS mRNA. A cell that has reduced expression of AASS may, for example, comprise a reduced amount of AASS protein. A cell that has reduced expression of AASS may, for example, comprise a reduced amount of AASS mRNA and a reduced amount of AASS protein.

The nucleic acid silencing molecule may, for example, reduce the expression of AASS in a cell contacted with the nucleic acid silencing molecule relative to a cell not contacted with the nucleic acid silencing molecule. The cell contacted with the nucleic acid silencing molecule may, for example, be human or from a human cell line. The cell contacted with the nucleic acid silencing molecule may, for example, be in vivo, ex vivo or in vitro. The cell not contacted with the nucleic acid silencing molecule may, for example, be human or from a human cell line. The cell not contacted with the nucleic acid silencing molecule may, for example, be in vivo, ex vivo or in vitro. Preferably, the cell contacted with the nucleic acid silencing molecule and the cell not contacted with the nucleic acid silencing molecule are the same type of cell (e.g. a human cell or a cell from a human cell line). Preferably, the cell contacted with the nucleic acid silencing molecule and the cell not contacted with the nucleic acid silencing molecule are under the same conditions (e.g. in vivo, ex vivo or in vitro). The cell not contacted with the nucleic acid silencing molecule may instead be contacted with a control molecule, such as a mock nucleic acid silencing molecule. Mock nucleic acid silencing molecules (such as mock antisense oligonucleotides) and methods for their design and production are well-known in the art. Reduced expression of AASS in a cell contacted with the nucleic acid silencing molecule may, for example, refer to a reduction in the amount of AASS mRNA and/or AASS protein comprised in the cell relative to a cell not contacted with the nucleic acid silencing molecule. Accordingly, a cell that is contacted with the nucleic acid silencing molecule and that has reduced expression of AASS may comprise a reduced amount of AASS mRNA relative to a cell not contacted with the nucleic acid silencing molecule. A cell that is contacted with the nucleic acid silencing molecule and that has reduced expression of AASS may comprise a reduced amount of AASS protein relative to a cell not contacted with the nucleic acid silencing molecule. A cell that is contacted with the nucleic acid silencing molecule and that has reduced expression of AASS may comprise a reduced amount of AASS mRNA and a reduced amount of AASS protein relative to a cell not contacted with the nucleic acid silencing molecule.

In any of the aspects described above, the nucleic acid silencing molecule may reduce the expression of AASS by at least 30%. In other words, the nucleic acid silencing molecule may reduce the amount of AASS mRNA and/or the amount of AASS protein by at least 30%. The nucleic acid silencing molecule may, for example, reduce the expression of AASS by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. In other words, the nucleic acid silencing molecule may, for example, reduce the amount of AASS mRNA and/or the amount of AASS protein by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. The nucleic acid silencing molecule may, for example, reduce the expression of AASS by 30% to 99%, such as 35% to 95%, 40% to 90%, 45% to 85%, 50% to 80%, 55% to 75%, or 60% to 70%. That is, the nucleic acid silencing molecule may, for example, reduce amount of AASS mRNA and/or the amount of AASS protein by 30% to 99%, such as 35% to 95%, 40% to 90%, 45% to 85%, 50% to 80%, 55% to 75%, or 60% to 70%.

Preferably, the nucleic acid silencing molecule reduces the expression of AASS in a cell by at least 30%. In other words, the nucleic acid silencing molecule may reduce the amount of AASS mRNA and/or the amount of AASS protein in a cell by at least 30%. Cell types and conditions for assessment of AASS expression are described above. The nucleic acid silencing molecule may, for example, reduce the expression of AASS in a cell by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. In other words, the nucleic acid silencing molecule may, for example, reduce the amount of AASS mRNA and/or the amount of AASS protein in a cell by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. The nucleic acid silencing molecule may, for example, reduce the expression of AASS in a cell by 30% to 99%, such as 35% to 95%, 40% to 90%, 45% to 85%, 50% to 80%, 55% to 75%, or 60% to 70%. That is, the nucleic acid silencing molecule may, for example, reduce amount of AASS mRNA and/or the amount of AASS protein in a cell by 30% to 99%, such as 35% to 95%, 40% to 90%, 45% to 85%, 50% to 80%, 55% to 75%, or 60% to 70%.

More preferably, the nucleic acid silencing molecule reduces the expression of AASS in a cell contacted with the nucleic acid silencing molecule by at least 30% relative to a cell not contacted with the nucleic acid silencing molecule. In other words, the nucleic acid silencing molecule may reduce the amount of AASS mRNA and/or the amount of AASS protein in a cell contacted with the nucleic acid silencing molecule by at least 30% relative to a cell not contacted with the nucleic acid silencing molecule. Cell types and conditions for assessment of AASS expression are described above. The nucleic acid silencing molecule may, for example, reduce the expression of AASS in a cell contacted with the nucleic acid silencing molecule by at least at least 30%, at least 35%, at least 40%, 45%, at least 50%, at least 55%, at least 60%, at least 65%, at least 70%, at least 75%, at least 80%, at least 85%, at least 90%, at least 95%, at least 98%, at least 99% relative to a cell not contacted with the nucleic acid silencing molecule. In other words, the nucleic acid silencing molecule may, for example, reduce the amount of AASS mRNA and/or the amount of AASS protein in a cell contacted with the nucleic acid silencing molecule by at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99% relative to a cell not contacted with the nucleic acid silencing molecule. The nucleic acid silencing molecule may, for example, reduce the expression of AASS in a cell contacted with the nucleic acid silencing molecule by 30% to 99%, such as 35% to 95%, 40% to 90%, 45% to 85%, 50% to 80%, 55% to 75%, or 60% to 70% relative to a cell not contacted with the nucleic acid silencing molecule. That is, the nucleic acid silencing molecule may, for example, reduce amount of AASS mRNA and/or the amount of AASS protein in a cell contacted with the nucleic acid silencing molecule by 30% to 99%, such as 35% to 95%, 40% to 90%, 45% to 85%, 50% to 80%, 55% to 75%, or 60% to 70% relative to a cell not contacted with the nucleic acid silencing molecule.

In any of the aspects described above, the nucleic acid silencing molecule may reduce the expression of AASS by 100%. In other words, the nucleic acid silencing molecule may completely eliminate the expression of AASS. The nucleic acid silencing molecule may, for example, completely eliminate the expression of AASS mRNA (i.e. reduce the expression of AASS mRNA by 100%). The nucleic acid silencing molecule may, for example, completely eliminate the expression of AASS protein (i.e. reduce the expression of AASS protein by 100%). The nucleic acid silencing molecule may, for example, completely eliminate the expression of AASS mRNA and AASS protein (i.e. reduce the expression of AASS mRNA and AASS protein by 100%).

The nucleic acid silencing molecule may, for example, completely eliminate the expression of AASS in a cell. Accordingly, the cell may not express AASS mRNA and/or AASS protein. The cell may, for example, be human or from a human cell line. The cell may, for example, be in vivo, ex vivo or in vitro. The cell may be a cell contacted with the nucleic acid silencing molecule.

The nucleic acid silencing molecule may, for example, reduce the expression of AASS in a cell contacted with the nucleic acid silencing molecule by 100% relative to a cell not contacted with the nucleic acid silencing molecule. The cell contacted with the nucleic acid silencing molecule may, for example, be human or from a human cell line. The cell contacted with the nucleic acid silencing molecule may, for example, be in vivo, ex vivo or in vitro. The cell not contacted with the nucleic acid silencing molecule may, for example, be human or from a human cell line. The cell not contacted with the nucleic acid silencing molecule may, for example, be in vivo, ex vivo or in vitro. Preferably, the cell contacted with the nucleic acid silencing molecule and the cell not contacted with the nucleic acid silencing molecule are the same type of cell (e.g. a human cell or a cell from a human cell line). Preferably, the cell contacted with the nucleic acid silencing molecule and the cell not contacted with the nucleic acid silencing molecule are under the same conditions (e.g. in vivo, ex vivo or in vitro). The cell not contacted with the nucleic acid silencing molecule may instead be contacted with a control molecule, such as a mock nucleic acid silencing molecule. As set out above, mock nucleic acid silencing molecules (such as mock antisense oligonucleotides) and methods for their design and production are well-known in the art.

### Nucleic Acid Silencing Molecule

In the context of the disclosure, a silencing molecule may be defined as a molecule that reduces or eliminates (i.e. knocks down) expression of a target gene. A silencing molecule may, for example, reduce the amount of the mRNA product of target gene. A silencing molecule may, for example, eliminate the mRNA product of target gene. A silencing molecule may, for example, reduce the amount of the protein product of target gene. A silencing molecule may, for example, eliminate the protein product of target gene. In the present disclosure, the target gene is AASS.

In the context of the disclosure, a nucleic acid silencing molecule may be defined as a silencing molecule that comprises or consists of one or more nucleic acids. The nucleic acid silencing molecule of the disclosure may comprise RNA. The nucleic acid silencing molecule of the disclosure may comprise DNA. The nucleic acid silencing molecule of the disclosure may comprise DNA and RNA. The nucleic acid silencing molecule of the disclosure may consist of RNA. The nucleic acid silencing molecule of the disclosure may consist of DNA. The nucleic acid silencing molecule of the disclosure may consist of DNA and RNA.

The nucleic acid silencing molecule may reduce or eliminate (i.e. knock down) expression of AASS by any mechanism known in the art. The nucleic acid silencing molecule may, for example, bind to a mRNA molecule encoded by the AASS gene to block its translation into protein. The nucleic acid silencing molecule may, for example, bind to a mRNA molecule encoded by the AASS gene to induce degradation (such as enzymatic degradation) of the mRNA. The nucleic acid silencing molecule may, for example, bind to DNA encoding the AASS gene to induce methylation of the DNA and/or its associated histones. The nucleic acid silencing molecule may, for example, bind to DNA encoding AASS to facilitate removal of all or part of the AASS gene by gene editing.

For example, the nucleic acid silencing molecule may comprise or consist of an antisense oligonucleotide (AON). The nucleic acid silencing molecule may comprise or consist of a small interfering RNA (siRNA). The nucleic acid silencing molecule may comprise or consist of a short hairpin RNA (shRNA). The nucleic acid silencing molecule may comprise or consist of a microRNA (miRNA). The nucleic acid silencing molecule may comprise or consist of a CRISPR guide RNA. Preferably, the nucleic acid silencing molecule comprises or consists of an antisense oligonucleotide (AON) or a small interfering RNA (siRNA).

The nucleic acid silencing molecule may be about 10 to about 15000 nucleotides in length, such as about 100 to about 14000, about 200 to about 13000, about 300 to about 12000, about 400 to about 11000, about 400 to about 10000, about 500 to about 9000, about 600 to about 8000, about 700 to about 7000, about 800 to about 6000, about 900 to about 5000, about 1000 to about 4000, or about 2000 to 3000 in length. Preferably, the nucleic acid silencing molecule is less than 100 (such as less than 95, less than 90, less than 85, less than 80, less than 75, less than 70, less than 65, less than 60, less than 55, less than 50, less than 45, less than 40, less than 35, less than 30, less than 25, less than 20, less than 15, or less than 10) nucleotides in length. The nucleic acid silencing molecule may, for example be about 10 to about 50 nucleotides in length. For example, the nucleic acid silencing molecule may be about 10 to about 40, about 10 to about 30, about 10 to about 20, about 20 to about 50, about 20 to about 40, about 20 to about 30, about 30 to about 50, or about 30 to about 40 nucleotides in length. Preferably, the nucleic acid molecule is about 10 to about 30 (such as about 10 to about 20, or about 20 to about 30) nucleotides in length. The nucleic acid molecule may, for example, be about 10, about 11, about 12, about 13, about 14, about 14, about 16, about 17, about 18, about 19, about 20, about 21, about 22, about 23, about 24, about 25, about 26, about 27, about 28, about 29 or about 30 nucleotides in length. The nucleic acid molecule may preferably be about 16 or about 20 nucleic acids in length. Typical lengths of antisense oligonucleotides (AONs), small interfering RNAs (siRNAs), short hairpin RNAs (shRNAs), microRNAs (miRNAs), and CRISPR guide RNAs are well-known in the art.

The nucleic acid silencing molecule may, for example, comprise one or more 2′-O-methoxyethylribose (MOE) modified nucleotides or consist of 2′-O-methoxyethylribose (MOE) modified nucleotides. The nucleic acid silencing molecule may, for example, comprise one or more 2′-O-methyl (2OMe) modified nucleotides or consist of 2′-O-methyl (2OMe) modified nucleotides. The nucleic acid silencing molecule may, for example, comprise one or more locked nucleic acid (LNA) modified nucleotides or consist of locked nucleic acid (LNA) modified nucleotides. The nucleic acid silencing molecule may, for example, comprise one or more nucleotide phosphorothioates or consist of nucleotide phosphorothioates. MOE modified nucleotides, 2OMe modified nucleotides, LNA modified nucleotides and nucleotide phosphorothioates are described in the art.

The nucleic acid silencing molecule may be capable of binding to the AASS gene or to the RNA encoded by the AASS gene. The nucleic acid silencing molecule may be capable of binding to part of the AASS gene or to part of the RNA encoded by the AASS gene. Binding may, for example, be effected by hybridisation.

The nucleic acid silencing molecule may be directed to the nucleic acid sequence of the AASS gene. For example, the nucleic acid silencing molecule may be directed to the DNA of SEQ ID NO: 9. The nucleic acid silencing molecule may be directed to RNA encoded by the AASS gene. For example, the nucleic acid silencing molecule may be directed to mRNA encoded by the AASS gene. The nucleic acid silencing molecule may be directed to RNA encoded by the DNA of SEQ ID NO: 9. For example, the nucleic acid silencing molecule may be directed to mRNA encoded by the DNA of SEQ ID NO: 9. The nucleic acid silencing molecule may be directed to the mRNA of SEQ ID NO: 10. The nucleic acid silencing molecule may be directed to a nucleic acid sequence encoding AASS protein. For example, the nucleic acid silencing molecule may be directed to a nucleic acid sequence encoding the AASS protein of SEQ ID NO: 11. The nucleic acid silencing molecule may be directed to nucleic acid sequence encoding a protein having at least 90% sequence identity to SEQ ID NO: 11. For instance, the nucleic acid silencing molecule may be directed to nucleic acid sequence encoding a protein having at least 91%, at least 92%, at least 93%, at least 94%, at least 95%, at least 96%, at least 97%, at least 98%, or at least 99% sequence identity to SEQ ID NO: 11. A nucleic acid silencing molecule that is “directed to” a particular nucleic acid sequence is capable of binding to (e.g. hybridising to) that nucleic acid sequence.

The nucleic acid silencing molecule may comprise (a) a nucleotide sequence that has at least 75% sequence identity to a nucleotide sequence comprised in a primary transcript of AASS or (b) a nucleotide sequence that is complementary to the nucleotide sequence of (a). The nucleic acid silencing molecule may, for example, comprise or consist of an antisense oligonucleotide (AON). In one aspect, the nucleic acid silencing molecule may comprise a nucleotide sequence that has at least 80%, at least 85%, at least 90%, at least 95%, at least 96%, at least 97%, at least 98%, at least 99%, or 100% sequence identity to a nucleotide sequence comprised in a primary transcript of AASS. In one aspect, the nucleic acid silencing molecule may comprise a nucleotide sequence that has 80% to 100%, 85% to 99%, 90% to 98%, or 95% to 97% sequence identity to a nucleotide sequence comprised in a primary transcript of AASS. In one aspect, the nucleic acid silencing molecule may comprise a nucleotide sequence that is complementary to a nucleotide sequence that has at least 80%, at least 85%, at least 90%, at least 95%, at least 96%, at least 97%, at least 98%, at least 99%, or 100% sequence identity to a nucleotide sequence comprised in a primary transcript of AASS. In one aspect, the nucleic acid silencing molecule may comprise a nucleotide sequence that is complementary to a nucleotide sequence that has 80% to 100%, 85% to 99%, 90% to 98%, or 95% to 97% sequence identity to a nucleotide sequence comprised in a primary transcript of AASS. A primary transcript is the single-stranded ribonucleic acid RNA product synthesised by transcription of DNA, that is processed to yield various mature RNA products such as mRNAs, tRNAs, and rRNAs. The primary transcript may, for example, be a precursor mRNA (pre-mRNA) that is processed to form mRNA. The nucleotide sequence comprised in the primary transcript may comprise one or more of (i) a nucleotide sequence comprised in an exon, (ii) a nucleotide sequence comprised in an intron, (iii) a nucleotide sequence comprised in a 3′ untranslated region, and (iv) a nucleotide sequence comprised in a 5′ untranslated region. The nucleotide sequence comprised in the primary transcript may, for example, comprise: (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv).

The nucleic acid silencing molecule may comprise (a) a nucleotide sequence that has at least 75% sequence identity to a nucleotide sequence comprised in a mRNA transcribed from AASS or (b) a nucleotide sequence that is complementary to the nucleotide sequence of (a). The nucleic acid silencing molecule may, for example, comprise or consist of an antisense oligonucleotide (AON). The nucleic acid silencing molecule may, for example, comprise or consist of a small interfering RNA (siRNA). In one aspect, the nucleic acid silencing molecule may comprise a nucleotide sequence that has at least 80%, at least 85%, at least 90%, at least 95%, at least 96%, at least 97%, at least 98%, at least 99%, or 100% sequence identity to a nucleotide sequence comprised in a mRNA transcribed from AASS. In one aspect, the nucleic acid silencing molecule may comprise a nucleotide sequence that has 80% to 100%, 85% to 99%, 90% to 98%, or 95% to 97% sequence identity to a nucleotide sequence comprised in a mRNA transcribed from AASS. In one aspect, the nucleic acid silencing molecule may comprise a nucleotide sequence that is complementary to a nucleotide sequence that has at least 80%, at least 85%, at least 90%, at least 95%, at least 96%, at least 97%, at least 98%, at least 99%, or 100% sequence identity to a nucleotide sequence comprised in a mRNA transcribed from AASS. In one aspect, the nucleic acid silencing molecule may comprise a nucleotide sequence that is complementary to a nucleotide sequence that has 80% to 100%, 85% to 99%, 90% to 98%, or 95% to 97% sequence identity to a nucleotide sequence comprised in a mRNA transcribed from AASS.

The nucleic acid silencing molecule may, for example, target exon 8 of AASS. The nucleic acid silencing molecule may, for example, target exon 9 of AASS. The nucleic acid silencing molecule may, for example, target exon 8 and exon 9 of AASS. Accordingly, the nucleic acid silencing molecule may comprise (a) a nucleotide sequence that has at least 75% sequence identity to a nucleotide sequence comprised in exon 8 and/or exon 9 of a mRNA transcribed from AASS or (b) a nucleotide sequence that is complementary to the nucleotide sequence of (a). That is, the nucleic acid silencing molecule may comprise (a) a nucleotide sequence that has at least 75% sequence identity to a nucleotide sequence comprised in exon 8 of a mRNA transcribed from AASS or (b) a nucleotide sequence that is complementary to the nucleotide sequence of (a). The nucleic acid silencing molecule may comprise (a) a nucleotide sequence that has at least 75% sequence identity to a nucleotide sequence comprised in exon 9 of a mRNA transcribed from AASS or (b) a nucleotide sequence that is complementary to the nucleotide sequence of (a). The nucleic acid silencing molecule may comprise (a) a nucleotide sequence that has at least 75% sequence identity to a nucleotide sequence comprised in exon 8 and exon 9 of a mRNA transcribed from AASS or (b) a nucleotide sequence that is complementary to the nucleotide sequence of (a). The nucleic acid silencing molecule may, for example, comprise or consist of an antisense oligonucleotide (AON). The nucleic acid silencing molecule may, for example, comprise or consist of a small interfering RNA (siRNA). In one aspect, the nucleic acid silencing molecule may comprise a nucleotide sequence that has at least 80%, at least 85%, at least 90%, at least 95%, at least 96%, at least 97%, at least 98%, at least 99%, or 100% sequence identity to a nucleotide sequence comprised in exon 8 and/or exon 9 of a mRNA transcribed from AASS. In one aspect, the nucleic acid silencing molecule may comprise a nucleotide sequence that has 80% to 100%, 85% to 99%, 90% to 98%, or 95% to 97% sequence identity to a nucleotide sequence comprised in exon 8 and/or exon 9 of a mRNA transcribed from AASS. In one aspect, the nucleic acid silencing molecule may comprise a nucleotide sequence that is complementary to a nucleotide sequence that has at least 80%, at least 85%, at least 90%, at least 95%, at least 96%, at least 97%, at least 98%, at least 99%, or 100% sequence identity to a nucleotide sequence comprised in exon 8 and/or exon 9 of a mRNA transcribed from AASS. In one aspect, the nucleic acid silencing molecule may comprise a nucleotide sequence that is complementary to a nucleotide sequence that has 80% to 100%, 85% to 99%, 90% to 98%, or 95% to 97% sequence identity to a nucleotide sequence comprised in exon 8 and/or exon 9 of a mRNA transcribed from AASS.

The nucleic acid silencing molecule may, for example, comprise or consist of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 34, SEQ ID NO: 35 or SEQ ID NO: 36. The nucleic acid silencing molecule may, for example, comprise or consist of a nucleotide sequence having at least 75% sequence identity to SEQ ID NO: 1, 2, 3, 4, 5, 6, 7, 8, 34, 35 or 36. For instance, the nucleic acid silencing molecule may comprise or consist of a nucleotide sequence having at least 80%, at least 85%, at least 90%, at least 95%, at least 96%, at least 97%, at least 98% or at least 99% sequence identity to SEQ ID NO: 1, 2, 3, 4, 5, 6, 7, 8, 34, 35 or 36. Preferably, the nucleic acid silencing molecule comprises or consists of: (a) SEQ ID NO: 4, or a nucleotide sequence having at least 75% sequence identity to SEQ ID NO: 4; (b) SEQ ID NO: 6, or a nucleotide sequence having at least 75% sequence identity to SEQ ID NO: 6; (c) SEQ ID NO: 7, or a nucleotide sequence having at least 75% sequence identity to SEQ ID NO: 7; (d) SEQ ID NO: 8, or a nucleotide sequence having at least 75% sequence identity to SEQ ID NO: 8; or (e) SEQ ID NO: 35 or a nucleotide sequence having at least 75% sequence identity to SEQ ID NO: 35.

SEQ ID NO: 7 is a 20mer that comprises the 16mer of SEQ ID NO: 4. SEQ ID NO: 8 is a 20mer that comprises the 16mer of SEQ ID NO: 6. SEQ ID NO: 4 and SEQ ID NO: 7 target exon 9 of AASS. SEQ ID NO: 6 and SEQ ID NO: 8 target exon 8 of AASS.

SEQ ID NO: 34 targets exon 9 of AASS. SEQ ID NO: 35 targets exon 8 of AASS. SEQ ID NO: 36 targets exon 8 of AASS.

SEQ ID NOs: 1, 2, 3, 4, 5, 6, 7, 8, 34, 35 and 36 contain the nucleic acid “N”. In each instance, N is independently selectable from T (thymine) and U (uracil). For each of SEQ ID NOs: 1, 2, 3, 4, 5, 6, 7, 8, 34, 35 and 36, any number of “N” may be “T”. For each of SEQ ID NOs: 1, 2, 3, 4, 5, 6, 7, 8, 34, 35 and 36, any number of “N” may be “U”. Each of SEQ ID NOs: 1, 2, 3, 4, 5, 6, 7, 8, 34, 35 and 36 may contain any combination of “T” and “U” in the positions designated “N”. Thus, each of SEQ ID NOs: 1, 2, 3, 4, 5, 6, 7, 8, 34, 35 and 36 may be a DNA sequence, a RNA sequence, or a hybrid DNA/RNA sequence.

A nucleotide sequence having at least 75% sequence identity to SEQ ID NO: 1, 2, 3, 4, 5, 6, 7, 8, 34, 35 or 36 may comprise one or more nucleotide substitutions with respect to SEQ ID NO: 1, 2, 3, 4, 5, 6, 7, 8, 34, 35 or 36 respectively. For example, the nucleotide sequence may comprise one, two, three or four substitutions with respect to SEQ ID NO: 1, 2, 3, 4, 5 or 6, which are 16mers. The nucleotide sequence may comprise one, two, three or four substitutions with respect to SEQ ID NO: 34 which is a 19mer. The nucleotide sequence may comprise one, two, three four, or five substitutions with respect to SEQ ID NO: 7, 8, 35 or 36, which are 20mers.

A nucleotide sequence having at least 75% sequence identity to SEQ ID NO: 1, 2, 3, 4, 5, 6, 7, 8, 34, 35 or 36 may comprise one or more nucleotide deletions with respect to SEQ ID NO: 1, 2, 3, 4, 5, 6, 7, 8, 34, 35 or 36 respectively. For example, the nucleotide sequence may comprise one, two, three or four deletions with respect to SEQ ID NO: 1, 2, 3, 4, 5 or 6, which are 16mers. The nucleotide sequence may comprise one, two, three or four deletions with respect to SEQ ID NO: 34 which is a 19mer. The nucleotide sequence may comprise one, two, three four, or five deletions with respect to SEQ ID NO: 7, 8, 35 or 36 which are 20mers.

A nucleic acid silencing molecule that comprises or consists of SEQ ID NO: 1 or a nucleotide sequence having at least 75% sequence identity thereto may, for example, comprise or consist of SEQ ID NO: 16 (AON1) or a nucleotide sequence having at least 75% sequence identity thereto, or SEQ ID NO: 24 (LNA-modified AON1) or a nucleotide sequence having at least 75% sequence identity thereto. Accordingly, the nucleic acid silencing molecule may comprise or consist of SEQ ID NO: 16 (AON1) or a nucleotide sequence having at least 75% sequence identity thereto, or SEQ ID NO: 24 (LNA-modified AON1) or a nucleotide sequence having at least 75% sequence identity thereto.

A nucleic acid silencing molecule that comprises or consists of SEQ ID NO: 2 or a nucleotide sequence having at least 75% sequence identity thereto may, for example, comprise or consist of SEQ ID NO: 17 (AON2) or a nucleotide sequence having at least 75% sequence identity thereto, or SEQ ID NO: 25 (LNA-modified AON2) or a nucleotide sequence having at least 75% sequence identity thereto. Accordingly, the nucleic acid silencing molecule may comprise or consist of SEQ ID NO: 17 (AON2) or a nucleotide sequence having at least 75% sequence identity thereto, or SEQ ID NO: 25 (LNA-modified AON2) or a nucleotide sequence having at least 75% sequence identity thereto.

A nucleic acid silencing molecule that comprises or consists of SEQ ID NO: 3 or a nucleotide sequence having at least 75% sequence identity thereto may, for example, comprise or consist of SEQ ID NO: 18 (AON3) or a nucleotide sequence having at least 75% sequence identity thereto, or SEQ ID NO: 26 (LNA-modified AON3) or a nucleotide sequence having at least 75% sequence identity thereto. Accordingly, the nucleic acid silencing molecule may comprise or consist of SEQ ID NO: 18 (AON3) or a nucleotide sequence having at least 75% sequence identity thereto, or SEQ ID NO: 26 (LNA-modified AON3) or a nucleotide sequence having at least 75% sequence identity thereto.

A nucleic acid silencing molecule that comprises or consists of SEQ ID NO: 4 or a nucleotide sequence having at least 75% sequence identity thereto may, for example, comprise or consist of SEQ ID NO: 19 (AON4) or a nucleotide sequence having at least 75% sequence identity thereto, SEQ ID NO: 27 (LNA-modified AON4) or a nucleotide sequence having at least 75% sequence identity thereto, or SEQ ID NO: 28 (MOE-modified AON4) or a nucleotide sequence having at least 75% sequence identity thereto. Accordingly, the nucleic acid silencing molecule may comprise or consist of SEQ ID NO: 19 (AON4) or a nucleotide sequence having at least 75% sequence identity thereto, SEQ ID NO: 27 (LNA-modified AON4) or a nucleotide sequence having at least 75% sequence identity thereto, or SEQ ID NO: 28 (MOE-modified AON4) or a nucleotide sequence having at least 75% sequence identity thereto.

A nucleic acid silencing molecule that comprises or consists of SEQ ID NO: 5 or a nucleotide sequence having at least 75% sequence identity thereto may, for example, comprise or consist of SEQ ID NO: 20 (AON5) or a nucleotide sequence having at least 75% sequence identity thereto, or SEQ ID NO: 29 (LNA-modified AON5) or a nucleotide sequence having at least 75% sequence identity thereto. Accordingly, the nucleic acid silencing molecule may comprise or consist of SEQ ID NO: 20 (AON5) or a nucleotide sequence having at least 75% sequence identity thereto, or SEQ ID NO: 29 (LNA-modified AON5) or a nucleotide sequence having at least 75% sequence identity thereto.

A nucleic acid silencing molecule that comprises or consists of SEQ ID NO: 6 or a nucleotide sequence having at least 75% sequence identity thereto may, for example, comprise or consist of SEQ ID NO: 21 (AON6) or a nucleotide sequence having at least 75% sequence identity thereto, SEQ ID NO: 30 (LNA-modified AON6) or a nucleotide sequence having at least 75% sequence identity thereto, or SEQ ID NO: 31 (MOE-modified AON6) or a nucleotide sequence having at least 75% sequence identity thereto. Accordingly, the nucleic acid silencing molecule may comprise or consist of SEQ ID NO: 21 (AON6) or a nucleotide sequence having at least 75% sequence identity thereto, SEQ ID NO: 30 (LNA-modified AON6) or a nucleotide sequence having at least 75% sequence identity thereto, or SEQ ID NO: 31 (MOE-modified AON6) or a nucleotide sequence having at least 75% sequence identity thereto.

A nucleic acid silencing molecule that comprises or consists of SEQ ID NO: 7 or a nucleotide sequence having at least 75% sequence identity thereto may, for example, comprise or consist of SEQ ID NO: 22 (AON7) or a nucleotide sequence having at least 75% sequence identity thereto, or SEQ ID NO: 32 (MOE-modified AON7) or a nucleotide sequence having at least 75% sequence identity thereto. Accordingly, the nucleic acid silencing molecule may comprise or consist of SEQ ID NO: 22 (AON7) or a nucleotide sequence having at least 75% sequence identity thereto, or SEQ ID NO: 32 (MOE-modified AON7) or a nucleotide sequence having at least 75% sequence identity thereto.

A nucleic acid silencing molecule that comprises or consists of SEQ ID NO: 8 or a nucleotide sequence having at least 75% sequence identity thereto may, for example, comprise or consist of SEQ ID NO: 23 (AON8) or a nucleotide sequence having at least 75% sequence identity thereto, or SEQ ID NO: 33 (MOE-modified AON8) or a nucleotide sequence having at least 75% sequence identity thereto. Accordingly, the nucleic acid silencing molecule may comprise or consist of SEQ ID NO: 23 (AON8) or a nucleotide sequence having at least 75% sequence identity thereto, or SEQ ID NO: 33 (MOE-modified AON8) or a nucleotide sequence having at least 75% sequence identity thereto.

SEQ ID NO: 22 is a 20mer that comprises the 16mer of SEQ ID NO: 19. SEQ ID NO: 23 is a 20mer that comprises the 16mer of SEQ ID NO: 21. SEQ ID NOs: 19, 22, 27, 28 and 32 target exon 9 of AASS. SEQ ID NOs: 21, 23, 30, 31 and 33 target exon 8 of AASS.

A nucleic acid silencing molecule that comprises or consists of SEQ ID NO: 34 or a nucleotide sequence having at least 75% sequence identity thereto may, for example, comprise or consist of SEQ ID NO: 37 (MOE7) or a nucleotide sequence having at least 75% sequence identity thereto, or SEQ ID NO: 40 (MOE-modified MOE7) or a nucleotide sequence having at least 75% sequence identity thereto. Accordingly, the nucleic acid silencing molecule may comprise or consist of SEQ ID NO: 37 (MOE7) or a nucleotide sequence having at least 75% sequence identity thereto, or SEQ ID NO: 40 (MOE-modified MOE7) or a nucleotide sequence having at least 75% sequence identity thereto.

A nucleic acid silencing molecule that comprises or consists of SEQ ID NO: 35 or a nucleotide sequence having at least 75% sequence identity thereto may, for example, comprise or consist of SEQ ID NO: 38 (MOE8) or a nucleotide sequence having at least 75% sequence identity thereto, or SEQ ID NO: 41 (MOE-modified MOE8) or a nucleotide sequence having at least 75% sequence identity thereto. Accordingly, the nucleic acid silencing molecule may comprise or consist of SEQ ID NO: 38 (MOE8) or a nucleotide sequence having at least 75% sequence identity thereto, or SEQ ID NO: 41 (MOE-modified MOE8) or a nucleotide sequence having at least 75% sequence identity thereto.

A nucleic acid silencing molecule that comprises or consists of SEQ ID NO: 36 or a nucleotide sequence having at least 75% sequence identity thereto may, for example, comprise or consist of SEQ ID NO: 39 (MOE9) or a nucleotide sequence having at least 75% sequence identity thereto, or SEQ ID NO: 42 (MOE-modified MOE9) or a nucleotide sequence having at least 75% sequence identity thereto. Accordingly, the nucleic acid silencing molecule may comprise or consist of SEQ ID NO: 39 (MOE9) or a nucleotide sequence having at least 75% sequence identity thereto, or SEQ ID NO: 42 (MOE-modified MOE9) or a nucleotide sequence having at least 75% sequence identity thereto.

A nucleotide sequence having at least 75% sequence identity to SEQ ID NO: 16, 17, 18, 19, 20, 21, 22, 23, 24, 25, 26, 27, 28, 29, 30, 31, 32, 33, 37, 38, 39, 40, 41 or 42 may comprise one or more nucleotide substitutions with respect to SEQ ID NO: 16, 17, 18, 19, 20, 21, 22, 23, 24, 25, 26, 27, 28, 29, 30, 31, 32, 33, 37, 38, 39, 40, 41 or 42 respectively. For example, the nucleotide sequence may comprise one, two, three or four substitutions with respect to SEQ ID NO: 16, 17, 18, 19, 20, 21, 24, 25, 26, 27, 28, 29, 30 or 31, which are 16mers. The nucleotide sequence may comprise one, two, three or four substitutions with respect to SEQ ID NO: 37 or 40 which are 19mers. The nucleotide sequence may comprise one, two, three, four, or five substitutions with respect to SEQ ID NO: 22, 23, 32, 33, 38, 39, 41 or 42 which are 20mers.

A nucleotide sequence having at least 75% sequence identity to SEQ ID NO: 16, 17, 18, 19, 20, 21, 22, 23, 24, 25, 26, 27, 28, 29, 30, 31, 32, 33, 37, 38, 39, 40, 41 or 42 may comprise one or more nucleotide deletions with respect to SEQ ID NO: 16, 17, 18, 19, 20, 21, 22, 23, 24, 25, 26, 27, 28, 29, 30, 31, 32, 33, 37, 38, 39, 40, 41 or 42 respectively. For example, the nucleotide sequence may comprise one, two, three or four deletions with respect to SEQ ID NO: 16, 17, 18, 19, 20, 21, 24, 25, 26, 27, 28, 29, 30 or 31, which are 16mers. The nucleotide sequence may comprise one, two, three or four deletions with respect to SEQ ID NO: 37 or 40 which are 19mers. The nucleotide sequence may comprise one, two, three four, or five deletions with respect to SEQ ID NO: 22, 23, 32, 33, 38, 39, 41 or 42 which are 20mers.

Any of SEQ ID NOs: 1, 2, 3, 4, 5, 6, 7, 8, 16, 17, 18, 19, 20, 21, 22, 23, 34, 35, 36, 37, 38 and 39 may comprise one or more (such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or more) 2′-O-methoxyethylribose (MOE) modified nucleotides or consist of 2′-O-methoxyethylribose (MOE) modified nucleotides. The 2′-O-methoxyethylribose (MOE) modified nucleotides may be present at any position(s) within SEQ ID NOs: 1, 2, 3, 4, 5, 6, 7, 8, 16, 17, 18, 19, 20, 21, 22, 23, 34, 35, 36, 37, 38 and 39.

Any of SEQ ID NOs: 1, 2, 3, 4, 5, 6, 7, 8, 16, 17, 18, 19, 20, 21, 22, 23, 34, 35, 36, 37, 38 and 39 may comprise one or more (such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or more) 2′-O-methyl (2OMe) modified nucleotides or consist of 2′-O-methyl (2OMe) modified nucleotides. The 2′-O-methyl (2OMe) modified nucleotides may be present at any position(s) within SEQ ID NOs: 1, 2, 3, 4, 5, 6, 7, 8, 16, 17, 18, 19, 20, 21, 22, 23, 34, 35, 36, 37, 38 and 39.

Any of SEQ ID NOs: 1, 2, 3, 4, 5, 6, 7, 8, 16, 17, 18, 19, 20, 21, 22, 23, 34, 35, 36, 37, 38 and 39 may comprise one or more (such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or more) locked nucleic acid (LNA) modified nucleotides or consist of locked nucleic acid (LNA) modified nucleotides. The LNA modified nucleotides may be present at any position(s) within SEQ ID NOs: 1, 2, 3, 4, 5, 6, 7, 8, 16, 17, 18, 19, 20, 21, 22, 23, 34, 35, 36, 37, 38 and 39.

Any of SEQ ID NOs: 1, 2, 3, 4, 5, 6, 7, 8, 16, 17, 18, 19, 20, 21, 22, 23, 34, 35, 36, 37, 38 and 39 may comprise one or more (such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or more) nucleotide phosphorothioates or consist of nucleotide phosphorothioates. The nucleotide phosphorothioate(s) may be present at any position(s) within SEQ ID NOs: 1, 2, 3, 4, 5, 6, 7, 8, 16, 17, 18, 19, 20, 21, 22, 23, 34, 35, 36, 37, 38 and 39.

Any of SEQ ID NOs: 1, 2, 3, 4, 5, 6, 7, 8, 16, 17, 18, 19, 20, 21, 22, 23, 34, 35, 36, 37, 38 and 39 may comprise any combination of (i) one or more 2OMe modified nucleotides, (ii) one or more LNA modified nucleotides, (iii) one or more 2′-O-methoxyethylribose (MOE) modified nucleotides and (iv) one or more nucleotide phosphorothioates. For example, any of SEQ ID NOs: 1, 2, 3, 4, 5, 6, 7, 8, 16, 17, 18, 19, 20, 21, 22, 23, 34, 35, 36, 37, 38 and 39 may comprise (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv).

For illustrative purposes, exemplary LNA modification of SEQ ID NOs: 16, 17, 18, 19, 20 and 21 is shown in SEQ ID NOs: 24, 25, 26, 27, 29 and 30 respectively. Exemplary nucleotide phosphorothioate modification of SEQ ID NOs: 16, 17, 18, 19, 20 and 21 is also shown in SEQ ID NOs: 24, 25, 26, 27, 29 and 30 respectively. Exemplary 2′-O-methoxyethylribose (MOE) modification of SEQ ID NOs: 19, 21, 22 and 23 is shown in SEQ ID NOs: 28, 31, 32 and 33 respectively. Exemplary nucleotide phosphorothioate modification of SEQ ID NOs: 19, 21, 22 and 23 is also shown in SEQ ID NOs: 28, 31, 32 and 33 respectively. Exemplary nucleotide phosphorothioate modification of SEQ ID NOs: 37, 38 and 39 is also shown in SEQ ID NOs: 40, 41 and 42 respectively. Exemplary 2′-O-methoxyethylribose (MOE) modification of SEQ ID NOs: 37, 38 and 39 is also shown in SEQ ID NOs: 40, 41 and 42 respectively. The exemplified modifications are not limiting. Any type of chemical modification (for example, 2OMe modification, LNA modification, 2′-O-methoxyethylribose (MOE) modification, nucleotide phosphorothioate modification) may be made at any or all of the exemplified positions. Chemical modifications (for example, 2OMe modification, LNA modification, 2′-O-methoxyethylribose (MOE) modification, nucleotide phosphorothioate modification) may also be made at non-exemplified positions.

In a preferred aspect, the nucleic acid silencing molecule comprises or consists of SEQ ID NO: 4 or a nucleotide sequence having at least 75% sequence identity thereto. In a more preferred aspect, the nucleic acid silencing molecule comprises or consists of SEQ ID NO: 19 (AON4) or a nucleotide sequence having at least 75% sequence identity thereto. The nucleic acid silencing molecule may, for example, comprise or consist of a chemically modified version of SEQ ID NO: 19 (or a nucleotide sequence having at least 75% sequence identity thereto), such as SEQ ID NO: 23 (or a nucleotide sequence having at least 75% sequence identity thereto) or (SEQ ID NO: 33 or a nucleotide sequence having at least 75% sequence identity thereto).

In another preferred aspect, the nucleic acid silencing molecule comprises or consists of SEQ ID NO: 7 or a nucleotide sequence having at least 75% sequence identity thereto. In a more preferred aspect, the nucleic acid silencing molecule comprises or consists of SEQ ID NO: 22 (AON7; a 20mer that comprises the 16mer of SEQ ID NO: 19 (AON4)) or a nucleotide sequence having at least 75% sequence identity thereto. The nucleic acid silencing molecule may, for example, comprise or consist of a chemically modified version of SEQ ID NO: 22 (or a nucleotide sequence having at least 75% sequence identity thereto), such as SEQ ID NO: 32 (or a nucleotide sequence having at least 75% sequence identity thereto).

In a further preferred aspect, the nucleic acid silencing molecule comprises or consists of SEQ ID NO: 6 or a nucleotide sequence having at least 75% sequence identity thereto. In a more preferred aspect, the nucleic acid silencing molecule comprises or consists of SEQ ID NO: 21 (AON6) or a nucleotide sequence having at least 75% sequence identity thereto. The nucleic acid silencing molecule may, for example, comprise or consist of a chemically modified version of SEQ ID NO: 21 (or a nucleotide sequence having at least 75% sequence identity thereto), such as SEQ ID NO: 30 (or a nucleotide sequence having at least 75% sequence identity thereto) or (SEQ ID NO: 31 or a nucleotide sequence having at least 75% sequence identity thereto).

In another preferred aspect, the nucleic acid silencing molecule comprises or consists of SEQ ID NO: 8 or a nucleotide sequence having at least 75% sequence identity thereto. In a more preferred aspect, the nucleic acid silencing molecule comprises or consists of SEQ ID NO: 23 (AON8; a 20mer that comprises the 16mer of SEQ ID NO: 21 (AON6)) or a nucleotide sequence having at least 75% sequence identity thereto. The nucleic acid silencing molecule may, for example, comprise or consist of a chemically modified version of SEQ ID NO: 23 (or a nucleotide sequence having at least 75% sequence identity thereto), such as SEQ ID NO: 33 (or a nucleotide sequence having at least 75% sequence identity thereto).

In another preferred aspect, the nucleic acid silencing molecule comprises or consists of SEQ ID NO: 35 or a nucleotide sequence having at least 75% sequence identity thereto. In a more preferred aspect, the nucleic acid silencing molecule comprises or consists of SEQ ID NO: 38 (MOE8) or a nucleotide sequence having at least 75% sequence identity thereto. The nucleic acid silencing molecule may, for example, comprise or consist of a chemically modified version of SEQ ID NO: 38 (or a nucleotide sequence having at least 75% sequence identity thereto), such as SEQ ID NO: 41 (or a nucleotide sequence having at least 75% sequence identity thereto).

### Conjugation

The nucleic acid silencing molecule may be conjugated to one or more non-nucleic acid moieties. For example, the nucleic acid silencing molecule may be conjugated to two or more, three or more, four or more, or five or more non-nucleic-acid moieties.

Preferably, the non-nucleic acid moiety is a delivery moiety. A delivery moiety is a molecule that assists in the in vivo delivery of a nucleic acid silencing molecule. For example, the delivery moiety may facilitate delivery of a nucleic acid silencing molecule to a target organ, tissue, or cell type. Any of the non-nucleic acid moieties mentioned below may function as a delivery moiety.

The non-nucleic acid moiety may, for example, be hydrophobic. The non-nucleic acid moiety may, for example, comprise a lipid. For example, the non-nucleic acid moiety may comprise cholesterol, a fatty acid, a triglyceride or a phospholipid. The fatty acid may, be saturated or unsaturated. The unsaturated fatty acid may be polyunsaturated. Preferably, the non-nucleic acid moiety comprises cholesterol or a polyunsaturated fatty acid.

The non-nucleic acid moiety may, for example, comprise a peptide, a polypeptide, or a protein. For example, the non-nucleic acid moiety may comprise a peptide ligand. The peptide or peptide ligand, may, for example, be a cognate ligand for a receptor present on the surface of a target cell, or a cell comprised in a target tissue or target organ. The non-nucleic acid moiety may, for example, comprise an antibody or antibody fragment. The antibody or antibody fragment, may, for example, be capable of binding to an antigen present on the surface of a target cell, or a cell comprised in a target tissue or organ. The antibody fragment may, for example, comprise or consist of a scFv, a Fab, a modified Fab, a Fab′, a modified Fab′, a F(ab′)2, or a scFv2.

The non-nucleic acid moiety may, for example, comprise a saccharide, disaccharide or polysaccharide. The non-nucleic acid moiety may, for example, comprise an amino sugar. Preferably, the non-nucleic acid moiety comprises N-Acetylgalactosamine (GalNAc). GalNAc is capable of binding to the asialoglycoprotein receptor (ASGPR), which is expressed on the surface of liver hepatocytes. Thus, by conjugating the nucleic acid silencing molecule to GalNAc, it is possible to deliver the nucleic acid silencing molecule specifically to liver hepatocytes. GalNAc is highly potent and has been demonstrated to dramatically increase the uptake of nucleic acid silencing molecules by hepatocytes, and to prolong its duration. Studies in mice have also suggested some biodistribution of GalNAc-conjugated oligonucleotides in the kidney. As lysine catabolism occurs primarily within the liver and then the kidney, conjugation of the nucleic acid silencing molecule to GalNAc is advantageous.

The non-nucleic acid moiety may, for example, comprise a nanoparticle. Suitable nanoparticles are known in the art. Methods for the production of such nanoparticles are also known. The nanoparticle may, for example, be a lipid nanoparticle, a liposome, polymeric nanoparticle, an inorganic nanoparticle, a virus-like particle (VLP), a self-assembling protein, a calcium phosphate nanoparticle, a silicon nanoparticle or a gold nanoparticle. Preferably, the nanoparticle is a lipid nanoparticle. Conjugation of a nucleic acid silencing molecule to a lipid nanoparticle may enhance the biodistribution and uptake of the nucleic acid silencing molecule in the liver and the kidney.

### Method of Treatment and Medical Use

Disclosed herein is a method of treating a disorder of the saccharopine pathway of lysine degradation in a subject, comprising administering to the subject a composition comprising a nucleic acid silencing molecule that reduces the expression of alpha-aminoadipic semialdehyde synthase (AASS).

Also disclosed herein is a composition for use in a method of treating a disorder of the saccharopine pathway of lysine degradation in a subject, wherein the composition comprises a nucleic acid silencing molecule that reduces the expression of alpha-aminoadipic semialdehyde synthase (AASS), and the method comprises administering the nucleic acid silencing molecule to the subject.

Administration of a nucleic acid silencing molecule that reduces the expression of AASS to a subject having a disorder of the saccharopine pathway of lysine degradation may block or inhibit the pathway at its early steps. In this way, the formation of downstream metabolites may be prevented or reduced. Accumulation of toxic metabolites, such as neurotoxic metabolites, may thus be prevented or reduced. By preventing or reducing the accumulation of toxic metabolites, disorders of the saccharopine pathway of lysine degradation may be treated. For example, clinical signs or symptoms of such disorders may be reduced or abolished. Clinical signs or symptoms may be stopped from progressing. The method of treatment and medical use disclosed herein may have advantages over other treatments for disorders of the saccharopine pathway of lysine degradation, such as dietary lysine restriction, as set out above.

### Nucleic Acid Silencing Molecule

The method of treatment and medical use described herein comprise administering to the subject a composition comprising a nucleic acid silencing molecule that reduces the expression of AASS. Such nucleic acid silencing molecules are described in detail above. Any of the aspects described above in connection with the nucleic acid silencing molecule of the disclosure may equally apply to the method of treatment of the disclosure or to the medical use of the disclosure.

The composition may comprise one or more nucleic acid silencing molecules that reduce the expression of AASS. For example, the composition may comprise two or more, five or more, ten or more, 20 or more, 50 or more, 100 or more, 200 or more, 500 or more, 1000 or more, 2000 or more, 5000 or more, 10000 or more, 20000 or more, 50000 or more, 100000 or more, 200000 or more, 500000 or more, 1000000 or more, 2000000 or more, 5000000 or more, 1×10⁷ or more, 2×10⁷ or more, 5×10⁷ or more, 1×10⁸ or more, 2×10⁸ or more, 5×10⁸ or more, 1×10⁹ or more, 2×10⁹ or more, or 5×10⁹ or more nucleic acid silencing molecules that reduce the expression of AASS per dose. Preferably, all of the more nucleic acid silencing molecules comprised in one dose of the composition comprise the same nucleotide sequence.

In one aspect, the nucleic acid silencing molecule may comprise one or more nucleotide phosphorothioates, or consist of nucleotide phosphorothioates. When the composition comprises two or more such nucleic acid silencing molecules, each of the two or more nucleic acid silencing molecules may be of one stereoisomer. Preferably, the composition comprises no further nucleic acid silencing molecules. This ensures the stereopurity of the composition. Stereopurity of nucleic acid silencing molecules is described in the art, for example in Iwamoto et al. (2017), Nature Biotechnology, 35:9, 845-851.

### Reducing the Expression of AASS

The nucleic acid silencing molecule comprised in the composition reduces the expression of AASS. Reduced expression of AASS is described in detail above in connection with the nucleic acid silencing molecule of the disclosure. Any of the aspects described in connection with the nucleic acid silencing molecule of the disclosure may equally apply to the method of treatment of the disclosure or to the medical use of the disclosure.

### Disorders of the Saccharopine Pathway of Lysine Degradation

Administration of a nucleic acid silencing molecule that reduces the expression of AASS blocks or inhibits the saccharopine pathway of lysine degradation at its early steps, because AASS is the first enzyme in the pathway. By blocking or inhibiting the top of the pathway upstream in this way, the formation of any downstream metabolites may be prevented or reduced. Therefore, any disorder of the saccharopine pathway of lysine degradation may be treated according to the method of treatment or medical use of the disclosure.

Preferably, the disorder is associated with the accumulation of toxic metabolites of the saccharopine pathway of lysine degradation. The toxic metabolites may, for example, be neurotoxic metabolites. Preferably, the disorder comprises a deficiency in one or more (such as two or more, three or more, or four or more) of the enzymes comprised in the saccharopine pathway of lysine degradation. The disorder may, for example, comprise a deficiency in ALDH7A1. The disorder may, for example, comprise a deficiency in DHTKD1. The disorder may, for example, comprise a deficiency in glutaryl-CoA dehydrogenase. The disorder may, for example, comprise a deficiency in AADAT.

The deficiency in the one or more enzymes may result from a mutation in the enzyme. For example, the deficiency in ALDH7A1 may result from a mutation in ALDH7A1. The deficiency in DHTKD1 may result from a mutation in DHTKD1. The deficiency in glutaryl-CoA dehydrogenase may result from a mutation in glutaryl-CoA dehydrogenase. The deficiency in AADAT may result from a mutation in AADAT. The mutation may comprise one or more substitutions in the enzyme. The mutation may comprise one or more insertions to the enzyme. The mutation may comprise one or more deletions from the enzyme.

The disorder may, for example, be pyridoxine-dependent epilepsy. Pyridoxine-dependent epilepsy results from a deficiency (e.g. a mutation) in ALDH7A1. The disorder may, for example, be is 2-aminoadipic 2-ketoadipic aciduria. 2-aminoadipic 2-ketoadipic aciduria (also known as 2-aminoadipic and 2-ketoadipic aciduria) results from a deficiency (e.g. a mutation) in DHTKD1. 2-aminoadipic 2-ketoadipic aciduria is also known as 2-aminoadipic 2-oxoadipic aciduria, or 2-aminoadipate and 2-oxoadipic aciduria. The disorder may, for example, be Charcot-Marie-Tooth disease type 2Q. Charcot-Marie-Tooth disease type 2Q results from a deficiency (e.g. a mutation) in DHTKD1. The disorder may, for example, be glutaric aciduria Type I. Glutaric aciduria Type I results from a deficiency (e.g. a mutation) in glutaryl-CoA dehydrogenase.

### Subject

The subject may, for example, be a mammal. The mammal may, for example, be a human or a non-human mammal such as a dog, cat, horse or farm animal. Preferably, the subject is a human.

The subject may, for example, be an adult. The subject may, for example, be a juvenile.

### Administration and Formulation

The composition may be administered by any route. Suitable routes include, but are not limited to, the intravenous, intrathecal, intracerebral ventricular, intramuscular, intraperitoneal, subcutaneous, intradermal, transdermal and oral/buccal routes.

The composition may comprise a delivery vehicle that optimises delivery of the nucleic acid silencing molecule in vivo. Suitable delivery vehicles are known in the art and include, for example, cell-targeting moieties, cell-penetrating moieties, lipids, lipoproteins, liposomes, lipoplexes, peptides, GalNAc, antibodies, aptamers, nanoparticles, exosomes, spherical nucleic acids, and DNA cages.

The compositions may be prepared together with a physiologically acceptable carrier or diluent. Typically, such compositions are prepared as liquid suspensions of nucleic acid silencing molecules and/or delivery vehicle-linked nucleic acid silencing molecules. The nucleic acid silencing molecules and/or delivery vehicle-linked nucleic acid silencing molecules may be mixed with an excipient which is pharmaceutically acceptable and compatible with the active ingredient. Suitable excipients are, for example, water, saline, dextrose, glycerol, of the like and combinations thereof. In addition, if desired, the pharmaceutical compositions may contain minor amounts of auxiliary substances such as wetting or emulsifying agents, and/or pH buffering agents.

The nucleic acid silencing molecules and/or delivery vehicle-linked nucleic acid silencing molecules are administered in a manner compatible with the dosage formulation and in such amount will be therapeutically effective. The quantity to be administered depends on the subject to be treated, the disease to be treated, and the capacity of the subject's immune system. Precise amounts of nucleic acid silencing molecules and/or delivery vehicle-linked nucleic acid silencing molecules required to be administered may depend on the judgement of the practitioner and may be peculiar to each subject.

As set out above, the nucleic acid silencing molecule may, in one aspect, be a CRISPR guide RNA. In this case, a CRISPR nuclease (such as Cas9, Cpf1, Cas12b, or CasX) may be administered to the subject. The CRISPR nuclease may be comprised in the composition comprising the nucleic acid silencing molecule, or in a separate composition. If the CRISPR nuclease is comprised in a separate composition, the composition comprising the nucleic acid silencing molecule and the composition comprising the CRISPR nuclease may be administered to the subject at the same time. The composition comprising the nucleic acid silencing molecule and the composition comprising the CRISPR nuclease may be administered to the subject at a different time. For example, the composition comprising the nucleic acid silencing molecule may be administered to the subject before the composition comprising the CRISPR nuclease. The composition comprising the nucleic acid silencing molecule may be administered to the subject after the composition comprising the CRISPR nuclease.

### Combination Therapy

The composition may be administered as part of a combination therapy. That is, the method of treatment or medical use may comprise administering to the subject a further therapeutic composition or a therapeutic regimen. Administration of a further composition or a therapeutic regimen may, for example, be desirable when the nucleic acid silencing molecule reduces rather than eliminates AASS expression. In some cases, though, reduction (rather than elimination) of AASS expression may be sufficient to effect treatment of the disorder.

The composition may be administered as part of a combination therapy in conjunction with any available therapeutic composition or therapeutic regimen for a particular disorder. For instance, many disorders of the saccharopine pathway of lysine degradation may be treated with a lysine-restricted diet. Thus, the therapeutic regimen may comprise a lysine restricted diet. Some disorders of the saccharopine pathway of lysine degradation (such as disorders comprising a deficiency in glutaryl-CoA dehydrogenase, e.g. glutaric aciduria Type I) may be treated with a tryptophan-restricted diet. Thus, the therapeutic regimen may comprise a tryptophan restricted diet. Vitamin B6 is a known treatment for pyridoxine-dependent epilepsy. Thus, when the disorder comprises a deficiency in ALDH7A1 (e.g. pyridoxine-dependent epilepsy) the further therapeutic composition may comprise vitamin B6. Another known treatment for disorders comprising a deficiency in ALDH7A1 is arginine. Thus, when the disorder comprises a deficiency in ALDH7A1, the further therapeutic composition may comprise arginine. Arginine is also a known treatment for glutaric aciduria Type I. Thus, when the disorder comprises a deficiency in glutaryl-CoA dehydrogenase (e.g. glutaric aciduria Type I), the further therapeutic composition may comprise arginine. Carnitine is a known treatment for glutaric aciduria Type I. Thus, when the disorder comprises a deficiency in glutaryl-CoA dehydrogenase (e.g. glutaric aciduria Type I), the further therapeutic composition may comprise carnitine.

In combination therapy, the composition that comprises a nucleic acid silencing molecule that reduces the expression of AASS is administered in such an amount that will be therapeutically effective in combination with administration of the further therapeutic composition or therapeutic regimen. The further therapeutic composition is administered, in such an amount that will be therapeutically effective in combination with the composition that comprises a nucleic acid silencing molecule that reduces the expression of AASS. The composition that comprises a nucleic acid silencing molecule that reduces the expression of AASS and the further therapeutic composition may be administered together, for instance at the same time. The composition that comprises a nucleic acid silencing molecule that reduces the expression of AASS and the further therapeutic composition may be administered separately, for instance at a different time. For example, the composition that comprises a nucleic acid silencing molecule that reduces the expression of AASS may be administered before the further therapeutic composition. The composition that comprises a nucleic acid silencing molecule that reduces the expression of AASS may be administered after the further therapeutic composition. Administration of the composition that comprises a nucleic acid silencing molecule that reduces the expression of AASS may be alternated with administration of the further therapeutic composition.

In another aspect of combination therapy, the composition that comprises a nucleic acid silencing molecule that reduces the expression of AASS is administered in such an amount that it will be therapeutically effective in combination with the additional therapeutic regimen. The therapeutic regimen is implemented to the extent that it will be therapeutically effective in combination with administration of the composition that comprises a nucleic acid silencing molecule that reduces the expression of AASS. The composition that comprises a nucleic acid silencing molecule that reduces the expression of AASS may be administered before, during or after implementation of the therapeutic regimen. Preferably, the composition that comprises a nucleic acid silencing molecule that reduces the expression of AASS is administered during implementation of the therapeutic regimen.

### In Vitro Method

As set out above, the nucleic acid silencing molecule disclosed herein may be used to reduce the expression of AASS in vitro. For example, the nucleic acid silencing molecule may be used as a research tool, for instance to investigate the saccharopine pathway of lysine degradation. Accordingly, the present disclosure provides an in vitro method for reducing expression of AASS in a cell, comprising contacting the cell with the nucleic acid silencing molecule of the disclosure.

The cell may be any type of cell. For example, the cell may be from any tissue. The cell may be from any species. Preferably, the cell is human. The cell may be a healthy cell, or a diseased cell. The diseased cell may, for example, be a cancer cell. The cell may be a naturally occurring cell. The cell may be from a cell line.

Mechanisms for contacting a cell with a nucleic acid silencing molecule are well known in the art. Contacting may, for example, take place in a well of a microwell plate, or in another type of vessel such as a cell culture flask or a test tube. The contacting step may be performed prior to, or concurrently with, culture of the cell. The conditions required for the culture of various cell types are well known in the art.

The cell may be contacted with one or more molecules additional to the nucleic acid silencing molecule. The additional molecule may be another nucleic acid silencing molecule that targets AASS. The additional molecule may be a nucleic acid silencing molecule that targets a gene other than AASS, such as a different gene within the saccharopine pathway of lysine degradation. Preferably, the additional molecule may facilitate reduction or elimination of AASS expression by the nucleic acid silencing molecule. For example, if the nucleic acid silencing molecule is a CRISPR guide RNA, the additional molecule may be a CRISPR nuclease. The CRISPR nuclease may, for example, be Cas9, Cpf1, Cas12b, or CasX.

The following Examples illustrate the invention.

### EXAMPLES

### Example 1

### Materials and Methods

### Patients

Fibroblast cell lines from five patients with ALDH7A1-deficiency and four healthy controls were established from skin biopsies.

Human Fibroblast Cell culture

All patient and control fibroblast cells were grown in Dulbecco's modified Eagle medium (DMEM) supplemented with 10% heat inactivated fetal bovine serum (FBS), 100 U/ml penicillin and 100 μg/ml streptomycin at 37° C. and 5% CO₂.

### Antisense Oligonucleotides (AONs) Design

AONs with Locked Nucleic Acid (LNA) (Qiagen) and 2′-O-methoxy-ethyl (MOE) (Eurogentec) chemistries were designed to target AASS (α-aminoadipic semialdehyde synthase) and synthesised commercially. The length of gapmer was 16 mers for LNA chemistry and 20 mers for MOE chemistry. The central DNA gap is phosphorothioate (PS) modified DNA, while the flanking RNA regions were modified with MOE or LNA.

### AON Treatment of Cells

Cells were seeded in a six-well plate at a density of 2×10⁵ in fibroblast growth medium (2 mL) and, on the following day when 85-90% confluent, were transfected according to the manufactures instructions with 5 μL Lipofectamine 2000 (ThermoFisher) containing AON for 10 mins prior to the addition of 1 ml Opti-MEM (ThermoFisher) for 24 hours. For each experiment three control conditions were used: cells treated only with lipofectamine (mock); cells treated with an AON with a scrambled sequence that does not bind to mRNA (scrambled); and cells treated only with fibroblast growth media.

Harvesting and preparation of cells for AASA analysis by mass spectrometry Media was removed from the cells after 24 hr treatment with the AON and cells were washed twice with PBS. Cells were then left to grown for an additional 24 hours in DMEM containing 400 mg/L L-Lysine. Conditioned media was then collected and cells were washed three times with cold PBS. 2400 μL ice-cold methanol was added to cells, followed by 2400 μL of Millipore cold H₂O, all steps were performed on ice. Cells were harvested using a cell scraper, transferred to a 1.5 mL eppendorf tube and sonicated for 30 seconds (40% amp) prior to centrifugation at 15000 rpm at 4° C. for 30 minutes. The supernatant was transferred to a new 1.5 mL Eppendorf tube and analysed immediately by mass spectrometry or stored at −80° C. prior to analysis.

Analysis of AASA metabolite using LC/MS-MS

FMOC-chloride, boric acid, acetone, ammonium acetate, formic acid, and acetonitrile were of HPLC-grade and purchased from Sigma-Aldrich. The deuterated internal standard, α-aminoadipic acid-d3 (AAA-d3, 98% purity), was purchased from CDN isotopes (Quebec, Canada). L-Allysine ethylene acetal was purchase from Sigma-Aldrich.

Supernatants were transferred into glass vials and dried under a stream of nitrogen prior to resuspension in 50 μL of water, 10 μL of 0.1 mmol AAA-d3 and 125 μL of borate buffer (pH 10.4). The sample was then derivatised by the addition of 125 μL of Fmoc-chloride (6 mM in acetone), thorough mixing, and allowing to react for 15 minutes at room temperature.

Quantitation of α-AASA was performed by Liquid Chromatography-Mass Spectrometry (LC-MS/MS) using a Waters Acquity UPLC coupled to a Waters Xevo TQ-S mass spectrometer. Ten microliters of the prepared sample were injected onto a Discovery HS F5-5 LC column (5 cm×2.1 mm×5 μM). The mobile phase consisted of A (4 mM ammonium acetate, pH 5.0 and B (100% acetonitrile) and the following gradient was used: 0-1.9 min (95% A and 5% B; 0.5 ml/min), 2-10 min (80% A and 20% B to 20% A and 80% B; 0.25 ml/min), 10-12 min (80% A and 20% B to 100% B; 0.25-0.5 ml/min), 12-16 min (100% B; 0.5 ml/min). The column was then re-equilibrated prior to the next sample injection. All gradient steps were linear. α-AASA and d3-AAA were analysed in negative ion mode. The multiple reaction transitions monitored were 366.1>144.1 m/z for α-AASA-FMOC (cone voltage=4V, collision voltage 20V) and 385.02>163.06 m/z for d3-AAA-FMOC (cone voltage=19V, collision voltage=13V).

An α-AASA standard curve was generated with concentrations of 0.032-0.1 mmol/L. The α-AASA standard was synthesised from L-Allysine ethylene acetal. Briefly, 5 mg L-Allysine ethylene acetal were added to 1 mL of dH₂O and then de-blocked by mixing with 15 mg Amberlyst 15 resin for 10 min at room temperature. The 1 mL solution was then transferred to a new vial, and the resin was mixed with 0.5 mL 25% ammonium hydroxide and 2 mL dH₂O. A Further 3.11 mL dH₂O were added to make up a 6.61 mL solution of 1 mmol/L α-AASA.

RNA Extraction and cDNA Preparation

Total RNA was extracted from cells using the RNeasy Plus Mini Kit according to manufacturer's instructions. The yield and quality of the RNA of each sample was determined by measuring the absorbance at 260 and 280 nm using a NanoDrop spectrophotometer. Total RNA (400-500 ng) was reverse transcribed, according to the manufacturer instructions, in a 20 μL reaction mixture using random hexamers and the SuperScript IV Master Mix cDNA Synthesis kit (ThermoFisher).

### Quantitative Real-Time PCR (RT-PCR) Analysis

Quantitative real-time PCR analysis was performed using Power SYBR™ Green PCR Master Mix (ThermoFisher). Optimized Primers for AASS and GAPDH genes are detailed below. The StepOne™ real-time PCR system (Applied Biosystems) was used for quantitative real-time PCR and analysis using the following method: holding stage 95° C. for 10 min, followed by 40 cycles of denaturation at 95° C. for 15 seconds and annealing at 60° C. for 1 min, with melt curve (step 1: 95° C. for 15 seconds, step 2: 60° C. for 1 minute and step 3: 95° C. for 15 seconds (+0.3° C.). The relative quantification was measured using the ΔΔCt method. AASS expression levels were normalised to GAPDH. Each sample was analysed in triplicate.

| Gene | Forward Primer | Reverse Primer |
|---|---|---|
| AASS | AACAGTGGTCGCCTCCTAAC | TTTGTGTGGTAATGCAGGGC |
| | (SEQ ID NO: 12) | (SEQ ID NO: 13) |
| GAPDH | GAAGGTGAAGGTCGGAGTCA | TTGAGGTCAATGAAGGGGTC |
| | (SEQ ID NO: 14) | (SEQ ID NO: 15) |

### Quantification and Statistical Analysis

Data was analysed using a two-tailed unpaired Student's t-test (GraphPad Prism 8 software). When multiple groups were compared, a one-way ANOVA test was performed. p<0.05 was considered significant. Data are presented as mean±SEM.

### Results

AASS mRNA Expression

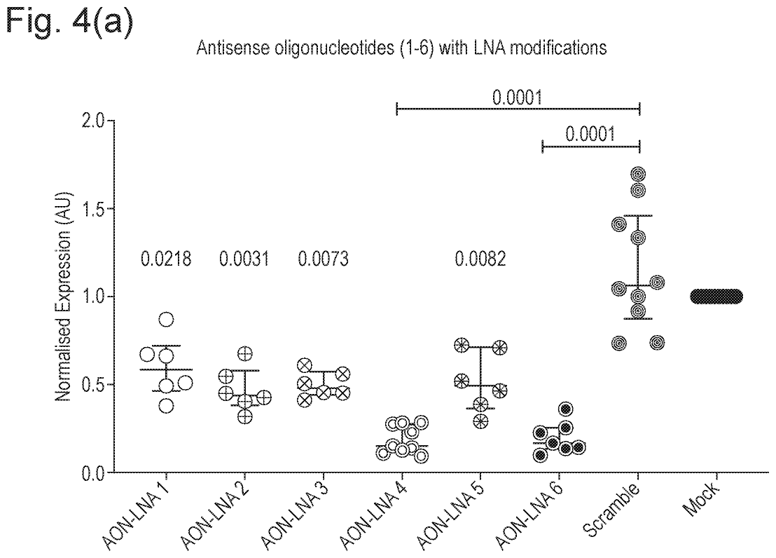

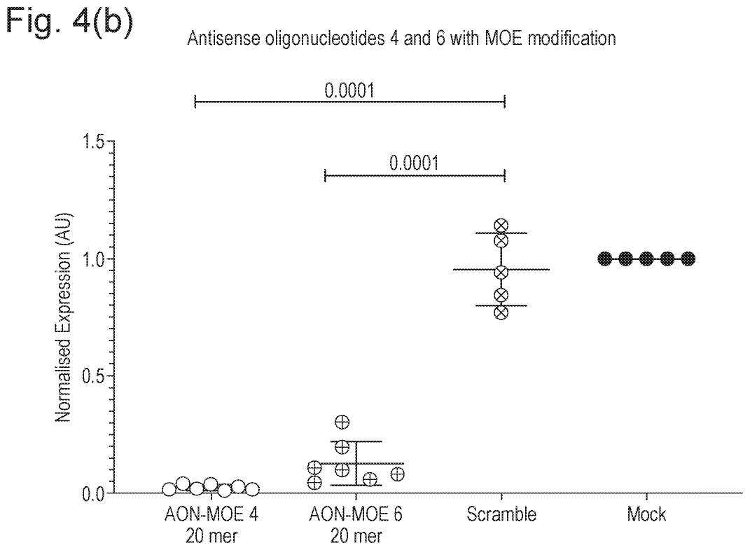

The effect of treatment with AASS-specific antisense oligonucleotide on AASS mRNA expression was assessed in ALDH7A1-deficient fibroblasts. The results are shown in FIG. 4. Both LNA modified and MOE modified antisense oligonucleotides reduced expression of AASS mRNA. In particular, LNA modified antisense oligonucleotides suppressed AASS (p<0.05), with AON4 and AON6 having the most dramatic effect (p=0.0001) (FIG. 4(a)). MOE-modified AON4 and AON6 (each in 20mer form, corresponding to AON7 and AON8 respectively) also significantly reduced AASS (p=0.0001) (FIG. 4(b)).

### Quantitation of AASA Metabolite

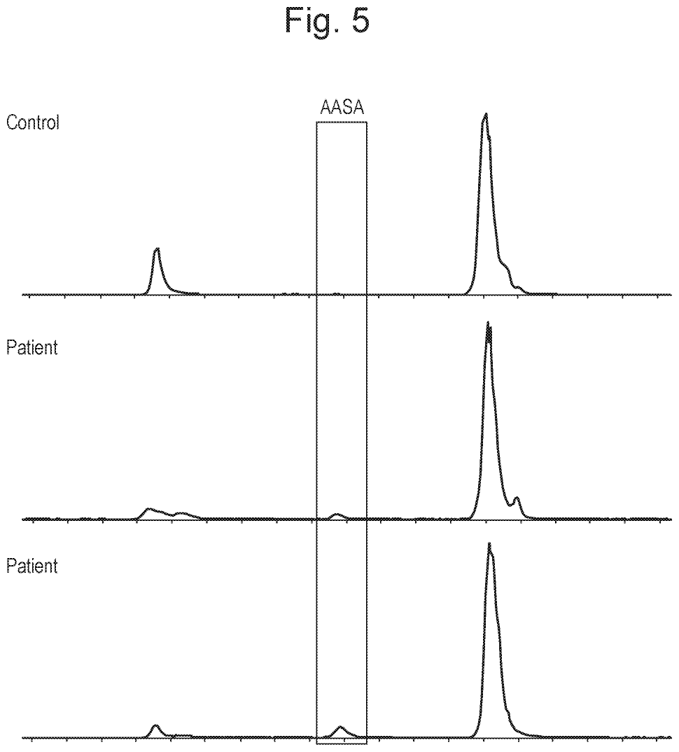

ALDH7A1-deficiency may be functionally assayed by measurement of AASA in fibroblasts using mass spectrometry. AASA is a metabolite of ALDH7A1, and ALDH7A1-deficiency may result in accumulation of AASA. This is confirmed in FIG. 5, which shows the results of mass spectrometry performed on fibroblasts from ALDH7A1 patients and from healthy controls. AASA was undetectable in fibroblasts from healthy controls, but an accumulation of AASA was demonstrated in fibroblasts from patients with ALDH7A1-deficiency.

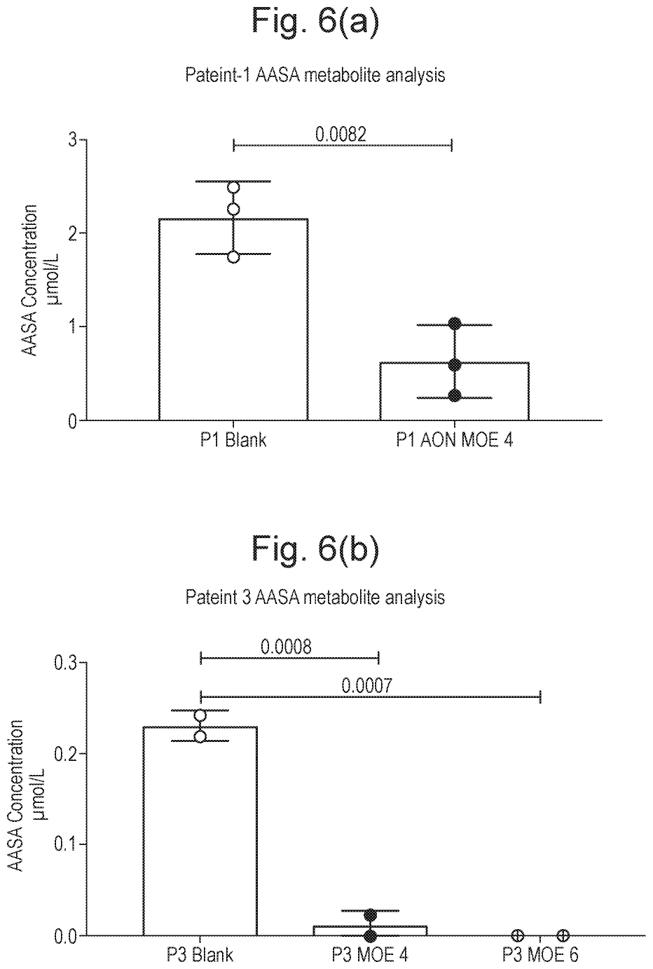

To determine the effect of antisense oligonucleotide treatment on AASS-driven production of AASA, patient fibroblasts were treated with AASS-specific antisense oligonucleotide or control antisense oligonucleotide. As shown in FIG. 6, the levels of AASA in fibroblasts from a) patient 1 (P1) and b) patient 3 (P3) are significantly reduced by the treatment with AON4 in MOE chemistry (p=0.0082 and p=0.0008, respectively). Similarly, AON6 in MOE chemistry also reduced AASA levels in P3 (p=0.0007). As set out above, the length of gapmer was 20 mers for MOE chemistry (i.e. AON7 was used as AON4 for MOE chemistry, and AON8 was used as AON6 for MOE chemistry).

AASS-specific antisense oligonucleotides may therefore be used to prevent or minimise the accumulation of metabolites caused by disorders of downstream enzymes in the saccharopine pathway of lysine degradation.

### Example 2

Design of Further AONs that Target Human AASS and Mouse Aass

Three new MOE modified antisense oligonucleotides (MOE 7, MOE 8 and MOE9) were designed to a specific region of the AASS gene, namely exon 8/exon 9. Greatest inhibition of AASS gene expression is achieved when this region is targeted. The human and mouse DNA sequence for this region of the gene are similar. MOE 7, MOE 8 and MOE9 are set out in Table 1.

TABLE 1
Target and sequence of AONs designed to
inhibit AASS

| Name | Target | AON antisense Sequence |
|---|---|---|
| MOE 7 | Human/Mouse | CCAGGAGACTCTGAGCATC |
| MOE 8 | Human/Mouse | CGACTTAACACCGTCCCATA |
| MOE 9 | Human/Mouse | TTCCTGACAAGATGATGATG |

Efficacy of AONs on Knockdown of AASS mRNA Expression in Human ALDH7A1-Deficient Fibroblasts

The potency of the following AONs was investigated in five ALDH7A1-deficient patient fibroblast cell lines:
MOE4 (which is AON4 in MOE chemistry and 20mer form, i.e. AON7 in MOE chemistry);
MOE6 (which is AON6 in MOE chemistry and 20mer form, i.e. AON8 in MOE chemistry);
MOE7
MOE8
MOE9.

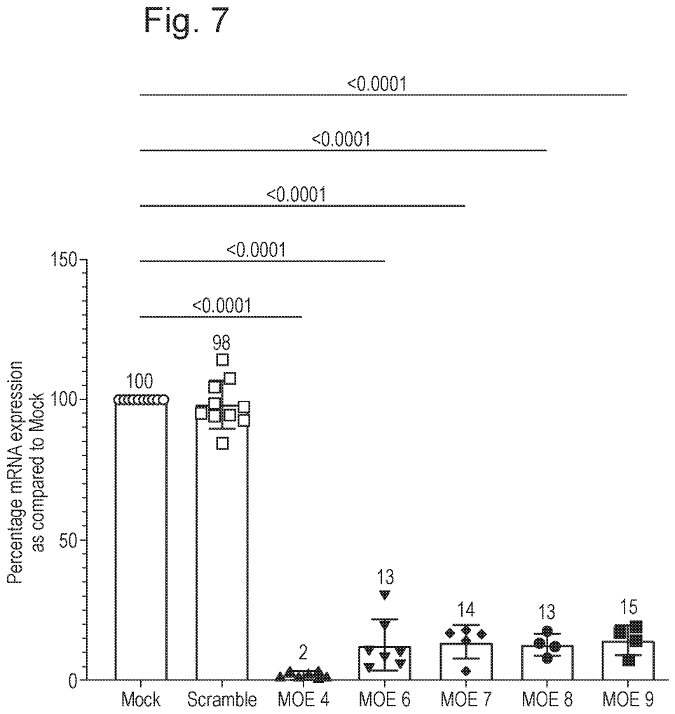

Potency was investigated by measuring levels of AASS mRNA expression by q-RT-PCR, 24 hours after transfection using lipofectamine 2000. All tested AONs decreased the levels of expression significantly, as shown in the FIG. 7 with up to 98% inhibition being achieved.

Efficacy of AONs on Knockdown of AASS mRNA Expression in Human GCDH-Deficient Fibroblasts (Glutaric Aciduria)

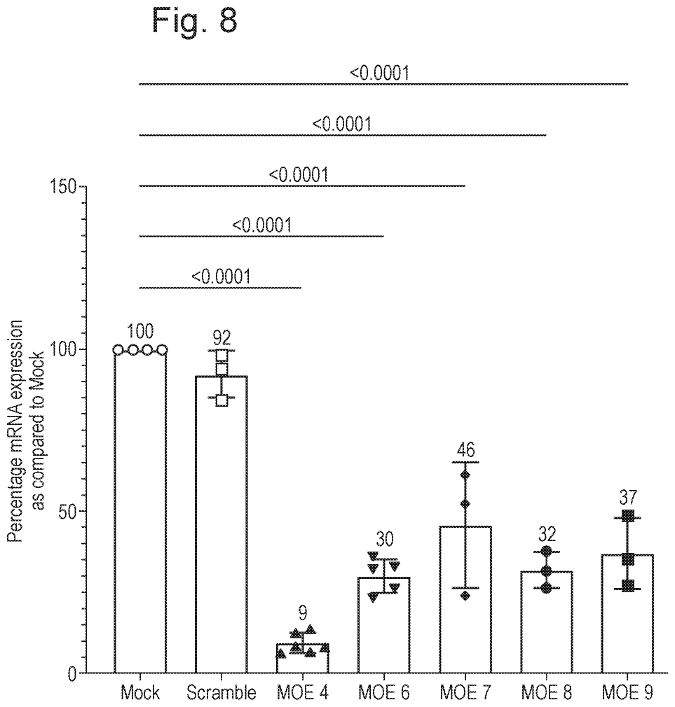

The effects of MOE 4, 6, 7, 8 and 9 were also analysed in two commercially available patient GCDH-deficient fibroblast cell lines, by measuring levels of AASS mRNA expression by q-RT-PCR, 24 hours after transfection using lipofectamine 2000. MOE 4, 6, 7, 8 and 9 were shown to inhibit expression significantly (FIG. 8) by up to 90%.

### Efficacy of AONs on Knockdown of Aass Gene Expression in Mouse Cell Lines

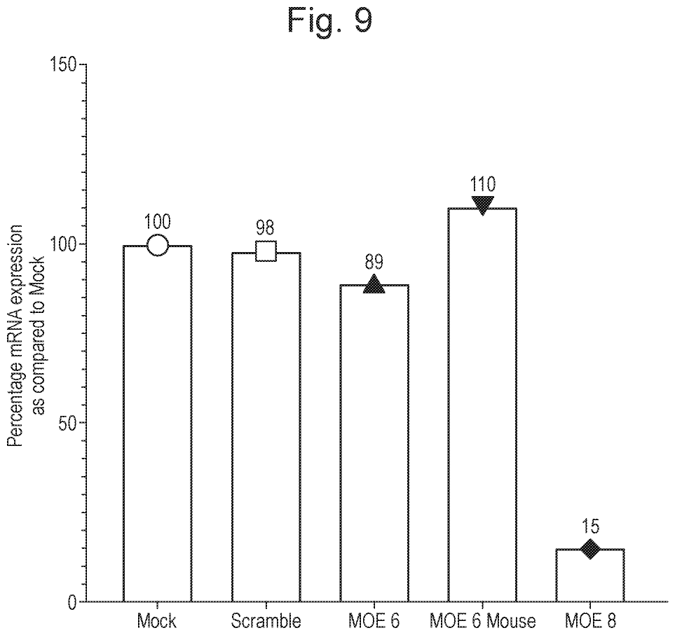

Aass expression was analysed in six mouse cell lines (Table 2). In five of these lines, expression of Aass was too low to give reliable results for Aass knockdown after treatment with AONs. It was possible, however, to investigate the effect of the AONs in the mouse fibroblast epithelial cell line 3T3-J2, where Aass is expressed at a much higher level. Greatest inhibition of Aass expression was evident with MOE 8, with approximately 85% knockdown being achieved by measuring levels of AASS mRNA expression by q-RT-PCR, 24 hours after transfection using lipofectamine 3000 (FIG. 9).

TABLE 2
Mouse cell lines used for Aass inhibition experiments

| Cell line | Origin | Ct Mean * |
|---|---|---|
| BLN CL.2 | Mouse liver epithelial cells | 32-34 |
| Neuro-2a | Mouse brain neuroblast | 32-34 |
| NIH-3T3 | Mouse embryo fibroblast | 29-32 |
| mIMCD3 | Mouse kidney epithelial | 31-34 |
| AML12 | Mouse hepatic epithelial | 32-35 |
| 3T3-J2 | Mouse embryo fibroblast | 26-27 |

*The lower the Ct value, the greater the amount of target nucleic acid. Ct values ≥ 29 indicate abundant nucleic acid whilst Ct values of 30-34 are considered weak positive reactions with low or moderate amounts of target nucleic acid.

### Development of a Functional Assay to Measure the Effect of the AONs in GA1-Deficient Patient Fibroblasts

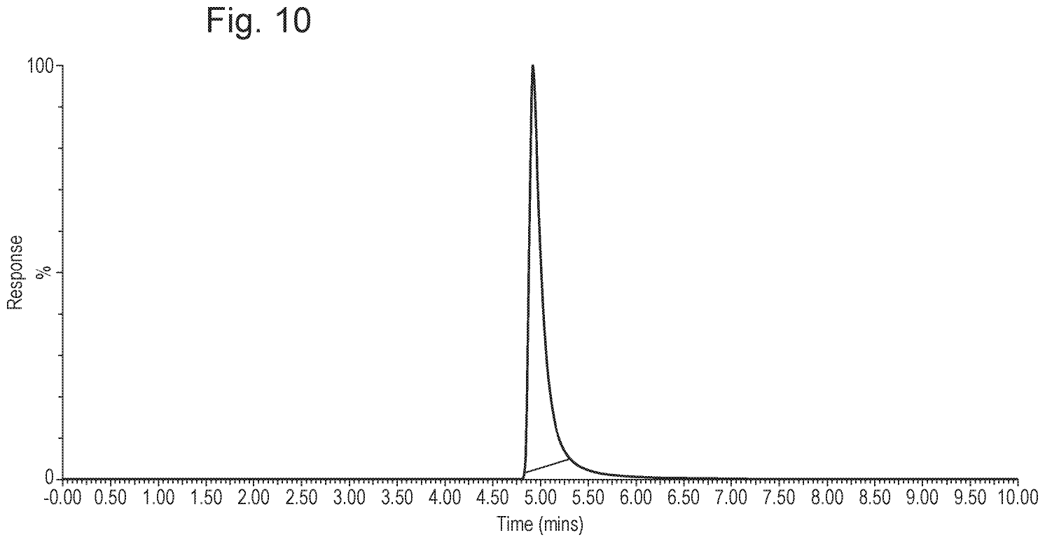

Glutaric aciduria type 1 (GA1) is caused by a deficiency in glutaryl-CoA dehydrogenase (GCDH), an enzyme involved in the metabolism of lysine and hydroxylysine. Mutations in GCDH result in accumulation of glutaric acid (GA), 3-OH-glutaric acid (3-OH-GA), glutaconic acid and glutarylcarnitine (C5DC). A mass spectrometry assay has been established to measure C5DC (FIG. 10). This will be used to functionally assess the effect of the AONs on GA1-deficient patient fibroblasts.

### Level of Expression of AASS in HEPG2 Human Liver Cells

Targeted delivery of AONs to liver cells (hepatocytes) will be achieved through conjugation of AONs with N-acetylgalactosamine (GalNAc) and their subsequent selective uptake by the asialoglycoprotein receptor (ASGR). Prior to looking at the effect of GalNAc conjugation on uptake, studies were performed to assess the level of AASS expression in a human HepG2 liver cell line. Cell lines that had undergone only a few passages (p=14) and those which had been passaged considerably more (p=78) were investigated to see if passage number affected expression level. Passage number did not affect level of AASS expression (Table 3).

TABLE 3
Levels of expression of AASS in HEPG2 cells

| Sample Name | Target mRNA | Ct Mean Values |
|---|---|---|
| HepG2 Passage 14 | AASS | 24.95 |
| HepG2 Passage 78 | AASS | 25.81 |

Transfection of HEPG2 Cells with AONs

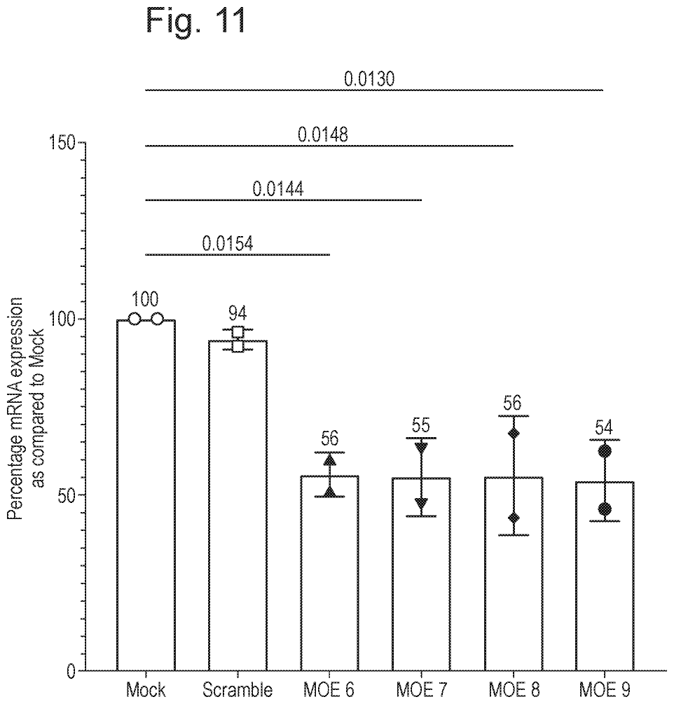

The potency of unconjugated AONs MOE 4, 6 and 8 was assessed after transfecting HEPG2 cells using the same methodology as had been used with the earlier human fibroblasts studies while using lipofectamine 3000 transfection reagent. Whilst a significant reduction in AASS mRNA expression was achieved (FIG. 11), using this approach we were unable to achieve a reduction as high as that seen in the patient fibroblast cells. This is likely to be due to a lower transfection efficiency caused by the HepG2 cells growing in clusters or on top of each other, unlike fibroblasts which grow in monolayers. In order to improve transfection, a reverse transfection method was adopted, allowing transfection to be performed prior to cells becoming attached to the plates.

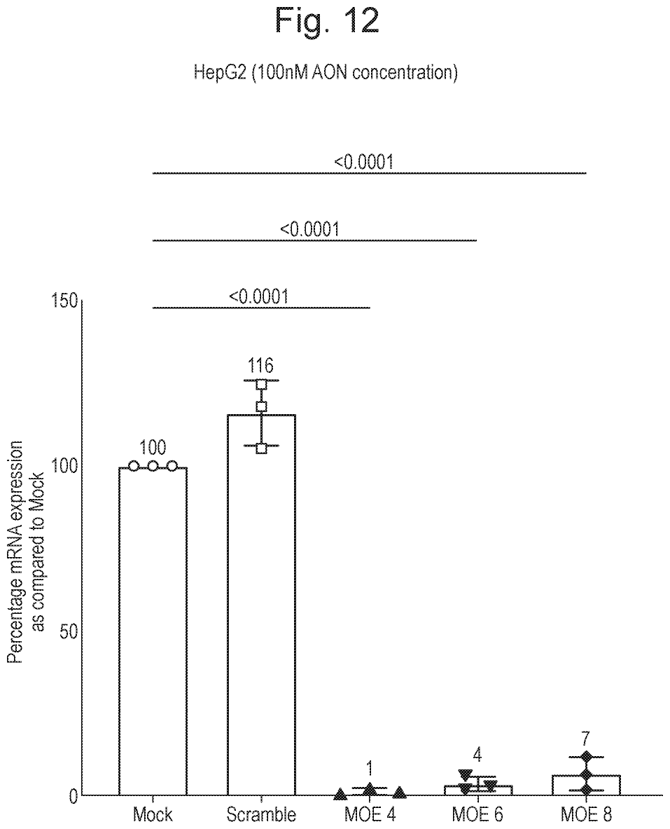

Conditions were optimised for reverse transfection of the HepG2 cells and the efficacy of unconjugated AONs 4, 6 and 8 were analysed using q-RT-PCR (FIG. 12; n=3).

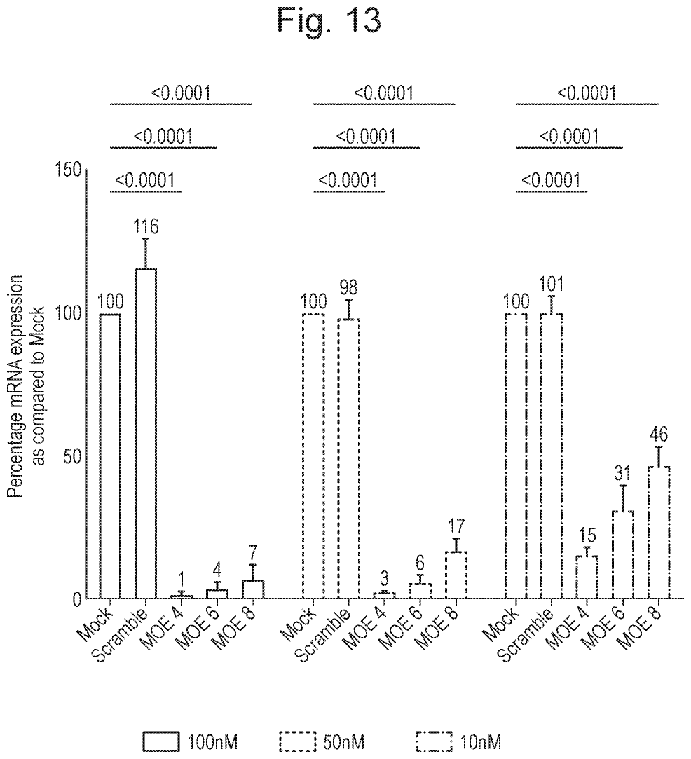

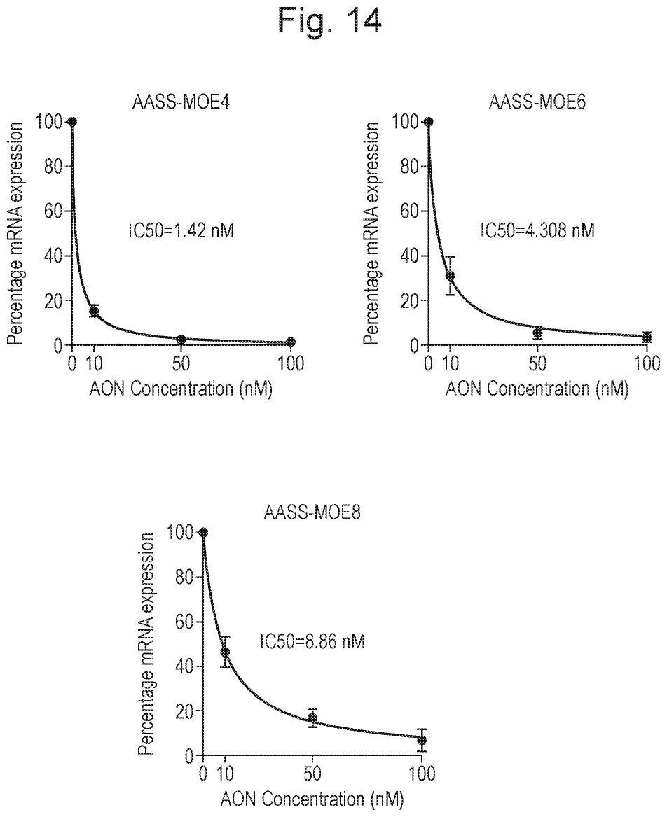

Dose response on a range of concentrations (10 nM, 50 nM, 100 nM) was performed. Results are shown in FIG. 13. Half-maximal inhibitory concentrations (IC50) of MOE 4, 6 and 8 vs, their corresponding control (mock treated) are 1.42 nM, 4.308 nM and 8.86 nM, respectively (FIG. 14).

### Methods

### Patient Cell Lines

Fibroblast cell lines and healthy controls were established from skin biopsies as for Example 1. Two GCDH-deficient fibroblast cell lines were purchased from Coriell Institute (Catalog ID's: GM16393 and GM16394).

### Cell Culture

All human patient and control fibroblasts cells, mouse epithelial fibroblasts (3T3 J2) and HepG2 cells were grown in Dulbecco's modified Eagle medium (DMEM) (ThermoFisher-41965039) supplemented with 10% heat inactivated Fetal Bovine Serum (FBS) (Gibco-10500064), 100 U/ml penicillin and 100 μg/ml streptomycin (ThermoFisher-15140122) at 37° C. and 5% CO₂. Cells were split when they reached approximately 75-80% confluency.

### Antisense Oligonucleotides (AONs) Design

AONs 2′-O-methoxy-ethyl (MOE) (Eurogentec) chemistries were designed to target AASS (α-aminoadipic semialdehyde synthase) and synthesised commercially. The AONs include:
MOE4 (which is AON4 in MOE chemistry and 20mer form, i.e. AON7 in MOE chemistry—SEQ ID NO: 32);
MOE6 (which is AON6 in MOE chemistry and 20mer form, i.e. AON8 in MOE chemistry—SEQ ID NO: 33);
MOE7 in MOE chemistry (SEQ ID NO: 40)
MOE8 in MOE chemistry (SEQ ID NO: 41)
MOE9 in MOE chemistry (SEQ ID NO: 42).

### AON Treatment of Fibroblast Cell Lines

AON treatment was performed as for Example 1.

### Reverse Transfection for HepG2 and 3T3 J2 Cell Lines

Lipofectamine 3000 (ThermoFisher—L3000001) was used as a transfection reagent with Opti-MEM media. Cells were counted and collected at a density of 5×10⁵ per 6 well plate in fibroblast growth medium (1.6 mL). 6 μL lipofectamine in 200 μL Opti-MEM media and the desired AON concentration in 200 μL Opti-MEM media were prepared, both lipofectamine and AONs were mixed and incubated for 15 minutes. In the next step, cells and AONs were mixed and transfected for 24 hours according to the manufacturer's instructions. For each experiment, three controls were used; a cell line treated only with lipofectamine i.e., mock control, a patient cell line treated with an AON with a scrambled sequence that does not bind to any mRNA, and a cell line treated only with fibroblast growth media i.e., blank control.

### RNA Extraction

Total RNA was extracted from cells using the Qiagen mini-RNA kit (Qiagen—74134) and the RNA integrity was assessed using the Agilent 2100 BioAnalyzer and determination of the RIN (RNA integrity number) scores for RNA quality control. Only samples with a good RNA purity were included in the studies.

cDNA Synthesis for RT-PCR

500 ng of total RNA was used for cDNA synthesis using SuperScript™ IV VILO Master Mix (ThermoFisher—11756050) according to the manufacturer's instructions.

### Quantitative Real-Time PCR (RT-PCR) Analysis

Quantitative real-time PCR analysis was performed using Power SYBR™ Green PCR Master Mix (ThermoFisher-4368577). Optimized Primers for human AASS and GAPDH and mouse Aass and Hprt1 genes are listed below. A StepOne™ real-time PCR system (Applied Biosystems) was used for quantitative real-time PCR as described in Example 1. The relative quantification was measured by ΔΔCt method using GAPDH/Hprt1 as the endogenous control or reference gene in order to normalize AASS/Aass expression levels. Each sample was tested at least three times.

| Gene | Forward Primer | Reverse Primer |
|---|---|---|
| AASS human | AACAGTGGTCGCCTCCTAAC | TTTGTGTGGTAATGCAGGGC |
| | (SEQ ID NO: 12) | (SEQ ID NO: 13) |
| GAPDH Human | GAAGGTGAAGGTCGGAGTCA | TTGAGGTCAATGAAGGGGTC |
| | (SEQ ID NO: 14) | (SEQ ID NO: 15) |
| Aass mouse | AGGGTCCAGATTCAAGAGCT | CTCTAAGCCTGGCCAGTCTC |
| | (SEQ ID NO: 43) | (SEQ ID NO: 44) |
| Hprt1 Mouse | CCCTGGTTAAGCAGTACAGC | ACAAAGTCTGGCCTGTATCCA |
| | (SEQ ID NO: 45) | (SEQ ID NO: 46) |

### Harvesting and Preparation of Cells for Metabolite Analysis by Mass Spectrometry

Adherent cells were harvested and prepared for metabolite analysis as described in Example 1.

### Analysis of AASA by Liquid-Chromatography Mass Spectrometry (LC-MS/MS)

AASA was analysed by LC-MS/MS as described for Example 1.

### Analysis of Glutarylcarnitine (C5DC) Using LC-MS/MS

Ammonium acetate, formic acid, and acetonitrile were of HPLC-grade and purchased from Sigma-Aldrich. Unlabelled internal standard C5DC (98% purity) was purchased from CDN isotopes (Quebec, Canada)

### Glutarylcarnitine LC-MS/MS Analysis

Quantitation of glutarylcarnitine was performed by using hydrophilic interaction liquid chromatography (HILIC)—Mass Spectrometry (MS) using a Waters Acquity UPLC coupled to a Waters Xevo TQ-S mass spectrometer. Five μL of the 50 μM unlabelled C5DC was analysed on a Acquity UPLC BEH Amide column 1.7 μm using mobile phase A of 10 mM ammonium acetate+0.2% formic acid and mobile phase B of 90% acetonitrile at a flow rate of 500 uL/min. C5DC was detected by multiple reaction monitoring in the positive ion mode using the following parameters: C5DC m/z 276.2>84.9 (cone voltage=30V, collision voltage 20V).

1. A method of treating a disorder of the saccharopine pathway of lysine degradation in a subject, comprising administering to the subject a composition comprising a nucleic acid silencing molecule that reduces the expression of alpha-aminoadipic semialdehyde synthase (AASS).

2. A composition for use in a method of treating a disorder of the saccharopine pathway of lysine degradation in a subject, wherein the composition comprises a nucleic acid silencing molecule that reduces the expression of alpha-aminoadipic semialdehyde synthase (AASS), and the method comprises administering the nucleic acid silencing molecule to the subject.

3. The method of claim 1, or composition for use of claim 2, wherein the disorder comprises a deficiency in ALDH7A1, DHTKD1, glutaryl-CoA dehydrogenase or AADAT.

4. The method or composition for use of claim 3, wherein the deficiency results from a mutation in ALDH7A1, DHTKD1, glutaryl-CoA dehydrogenase or AADAT respectively.

5. The method or composition for use of claim 4, wherein the mutation comprises one or more substitutions in, one or more insertions to, and/or one or more deletions from ALDH7A1, DHTKD1, glutaryl-CoA dehydrogenase or AADAT respectively.

6. The method of any one of claims 1 and 3 to 5, or composition for use of any one of claims 2 to 5, wherein:
(a) the disorder comprises a deficiency in ALDH7A1 and the disorder is pyridoxine-dependent epilepsy;
(b) the disorder comprises a deficiency in DHTKD1 and the disorder is 2-aminoadipic 2-ketoadipic aciduria or Charcot-Marie-Tooth disease type 2Q; or
(c) the disorder comprises a deficiency in glutaryl-CoA dehydrogenase and the disorder is glutaric aciduria Type I.

7. The method of any one of claims 1 and 3 to 6, or the composition for use of any one of claims 2 to 6, wherein the method comprises administering to the subject a further therapeutic composition or a therapeutic regimen.

8. The method or composition for use of claim 7, wherein (a) the disorder comprises a deficiency in ALDH7A1 and the further therapeutic composition comprises vitamin B6 and/or arginine; or (b) the disorder comprises a deficiency in glutaryl-CoA dehydrogenase and the further therapeutic composition comprises arginine and/or carnitine.

9. The method or composition for use of claim 7, wherein the therapeutic regimen comprises a therapeutic diet, optionally wherein the therapeutic diet comprises a lysine-restricted diet and/or a tryptophan restricted diet.

10. A nucleic acid silencing molecule that reduces the expression of alpha-aminoadipic semialdehyde synthase (AASS).

11. The method of any one of claims 1 and 3 to 9; the composition for use of any one of claims 2 to 9; or the nucleic acid silencing molecule of claim 10; wherein the nucleic acid silencing molecule comprises (i) RNA, (ii) DNA, or (iii) RNA and DNA.

12. The method of any one of claims 1, 3 to 9 and 11; the composition for use of any one of claims 2 to 9 and 11; or the nucleic acid silencing molecule of claim 10 or 11; wherein the nucleic acid silencing molecule comprises or consists of an antisense oligonucleotide (AON), a small interfering RNA (siRNA), a short hairpin RNA (shRNA), a microRNA (miRNA), or a CRISPR guide RNA; optionally wherein the nucleic acid silencing molecule comprises or consists of an antisense oligonucleotide (AON) or a small interfering RNA (siRNA).

13. The method of any one of claims 1, 3 to 9, 11 and 12; the composition for use of any one of claims 2 to 9, 11 and 12; or the nucleic acid silencing molecule of any one of claims 10 to 12; wherein the nucleic acid silencing molecule comprise one or more 2′-O-methoxyethylribose (MOE) modified nucleotides or consists of 2′-O-methoxyethylribose (MOE) modified nucleotides.

14. The method of any one of claims 1, 3 to 9, and 11 to 13; the composition for use of any one of claims 2 to 9 and 11 to 13; or the nucleic acid silencing molecule of any one of claims 10 to 13; wherein the nucleic acid silencing molecule comprises one or more 2′-O-methyl (2OMe) modified nucleotides or consists of 2′-O-methyl (2OMe) modified nucleotides.

15. The method of any one of claims 1, 3 to 9, and 11 to 14; the composition for use of any one of claims 2 to 9 and 11 to 14; or the nucleic acid silencing molecule of any one of claims 10 to 14; wherein the nucleic acid silencing molecule comprises one or more locked nucleic acid (LNA) modified nucleotides or consists of locked nucleic acid (LNA) modified nucleotides.

16. The method of any one of claims 1, 3 to 9, and 11 to 15; the composition for use of any one of claims 2 to 9 and 11 to 15; or the nucleic acid silencing molecule of any one of claims 10 to 15; wherein the nucleic acid silencing molecule comprises one or more nucleoside phosphorothioates or consists of nucleoside phosphorothioates.

17. The method or composition for use of claim 16, wherein (i) the composition comprises two or more nucleic acid silencing molecules as defined in claim 16; (ii) each of the two or more nucleic acid silencing molecules are of one stereoisomer; and (iii) no further nucleic acid silencing molecules are comprised in the composition.

18. The method of any one of claims 1, 3 to 9, and 11 to 17; the composition for use of any one of claims 2 to 9, and 11 to 17; or the nucleic acid silencing molecule of any one of claims 10 to 16; wherein the nucleic acid silencing molecule comprises (a) a nucleotide sequence that has at least 75% sequence identity to a nucleotide sequence comprised in a primary transcript of AASS or (b) a nucleotide sequence that is complementary to the nucleotide sequence of (a), optionally wherein the nucleic acid silencing molecule is an antisense oligonucleotide (AON).

19. The method, composition for use, or nucleic acid silencing molecule of claim 18; wherein the nucleotide sequence comprised in the primary transcript comprises one or more of (i) a nucleotide sequence comprised in an exon, (ii) a nucleotide sequence comprised in an intron, (iii) a nucleotide sequence comprised in a 3′ untranslated region, and (iv) a nucleotide sequence comprised in a 5′ untranslated region.

20. The method of any one of claims 1, 3 to 9, and 11 to 17; the composition for use of any one of claims 2 to 9, and 11 to 17; or the nucleic acid silencing molecule of any one of claims 10 to 16; wherein the nucleic acid silencing molecule comprises (a) a nucleotide sequence that has at least 75% sequence identity to a nucleotide sequence comprised in a mRNA transcribed from AASS or (b) a nucleotide sequence that is complementary to the nucleotide sequence of (a), optionally wherein the nucleic acid silencing molecule is an antisense oligonucleotide (AON) or a small interfering RNA (siRNA).

21. The method, composition for use, or nucleic acid silencing molecule of any one of claims 18 to 20, wherein the nucleic acid silencing molecule comprises (a) a nucleotide sequence that has at least 75% sequence identity to a nucleotide sequence comprised in exon 8 and/or exon 9 of a mRNA transcribed from AASS or (b) a nucleotide sequence that is complementary to the nucleotide sequence of (a).

22. The method of any one of claims 1, 3 to 9, and 11 to 21; the composition for use of any one of claims 2 to 9, and 11 to 21; or the nucleic acid silencing molecule of any one of claims 10 to 16 and 18 to 21, wherein the nucleotide sequence comprised in the nucleic acid silencing molecule is about 10 to about 15000 nucleotides in length, optionally about 10 to about 50 nucleotides in length or about 20 to about 30 nucleotides in length.

23. The method of any one of claims 1, 3 to 9, and 11 to 22; the composition for use of any one of claims 2 to 9, and 11 to 22; or the nucleic acid silencing molecule of any one of claims 11 to 17 and 19 to 22; wherein the nucleic acid silencing molecule comprises or consists of:
(a) SEQ ID NO: 7 or a nucleotide sequence having at least 75% sequence identity thereto, optionally wherein the nucleic acid silencing molecule comprises or consists of SEQ ID NO: 22 or SEQ ID NO: 32 or a nucleotide sequence having at least 75% sequence identity thereto; or
(b) SEQ ID NO: 8 or a nucleotide sequence having at least 75% sequence identity thereto, optionally wherein the nucleic acid silencing molecule comprises or consists of SEQ ID NO: 23 or SEQ ID NO: 33 or a nucleotide sequence having at least 75% sequence identity thereto; or
(c) SEQ ID NO: 35 or a nucleotide having at least 75% sequence identity thereto, optionally wherein the nucleic acid silencing molecule comprises or consists of SEQ ID NO: 38 or SEQ ID NO: 41 or a nucleotide sequence having at least 75% sequence identity thereto
(d) SEQ ID NO: 4 or a nucleotide sequence having at least 75% sequence identity thereto, optionally wherein the nucleic acid silencing molecule comprises or consists of SEQ ID NO: 19, SEQ ID NO: 27 or SEQ ID NO: 28 or a nucleotide sequence having at least 75% sequence identity thereto; or
(e) SEQ ID NO: 6 or a nucleotide sequence having at least 75% sequence identity thereto, optionally wherein the nucleic acid silencing molecule comprises or consists of SEQ ID NO: 21, SEQ ID NO: 30, or SEQ ID NO: 31 or a nucleotide sequence having at least 75% sequence identity thereto; or
(f) SEQ ID NO: 34 or a nucleotide sequence having at least 75% sequence identity thereto, optionally wherein the nucleic acid silencing molecule comprises or consists of SEQ ID NO: 37 or SEQ ID NO: 41 or a nucleotide sequence having at least 75% sequence identity thereto; or
(g) SEQ ID NO: 36 or a nucleotide sequence having at least 75% sequence identity thereto, optionally wherein the nucleic acid silencing molecule comprises or consists of SEQ ID NO: 39 or SEQ ID NO: 42 or a nucleotide sequence having at least 75% sequence identity thereto.
(h) SEQ ID NO: 1 or a nucleotide sequence having at least 75% sequence identity thereto, optionally wherein the nucleic acid silencing molecule comprises or consists of SEQ ID NO: 16 or SEQ ID NO: 24 or a nucleotide sequence having at least 75% sequence identity thereto; or
(i) SEQ ID NO: 2 or a nucleotide sequence having at least 75% sequence identity thereto, optionally wherein the nucleic acid silencing molecule comprises or consists of SEQ ID NO: 17 or SEQ ID NO: 25 or a nucleotide sequence having at least 75% sequence identity thereto; or
(j) SEQ ID NO: 3 or a nucleotide sequence having at least 75% sequence identity thereto, optionally wherein the nucleic acid silencing molecule comprises or consists of SEQ ID NO: 18 or SEQ ID NO: 26 or a nucleotide sequence having at least 75% sequence identity thereto; or
(k) SEQ ID NO: 5 or a nucleotide sequence having at least 75% sequence identity thereto, optionally wherein the nucleic acid silencing molecule comprises or consists of SEQ ID NO: 20 or SEQ ID NO: 29 or a nucleotide sequence having at least 75% sequence identity thereto.

24. The method of any one of claims 1, 3 to 9, and 11 to 23; the composition for use of any one of claims 2 to 9, and 11 to 23; or the nucleic acid silencing molecule of any one of claims 11 to 17 and 19 to 23; wherein the nucleic acid silencing molecule is conjugated to one or more non-nucleic acid moieties.

25. An in vitro method for reducing expression of AASS in a cell, comprising contacting the cell with the nucleic acid silencing molecule of any one of claims 11 to 16 and 18 to 24.
